(19) United States
(12) Patent Application Publication
(10) Pub. No.: US 2024/0034739 A1
(43) Pub. Date: Feb. 1, 2024

(54) PROCESSES FOR MAKING BICYCLIC KETONE COMPOUNDS

(71) Applicants: Genentech, Inc., South San Francisco, CA (US); Hoffmann-La Roche Inc., Little Falls, NJ (US)

(72) Inventors: Allen Yu HONG, South San Francisco, CA (US); Nicholas WONG, Dublin, CA (US); Sean M. KELLY, San Mateo, CA (US); Lauren Elizabeth SIROIS, San Francisco, CA (US); Sara SCHWEGLER, Mühlau (CH); Xin LINGHU, Burlingame, CA (US); Francis GOSSELIN, San Mateo, CA (US); Katarzyna Aleksandra PIECHOWICZ, Redwood City, CA (US); Carmela MOLINARO, Burlingame, CA (US); Dennis WETZL, Riehen (CH)

(73) Assignees: Genentech, Inc., South San Francisco, CA (US); Hoffmann-La Roche Inc., Little Falls, NJ (US)

(21) Appl. No.: 18/479,596

(22) Filed: Oct. 2, 2023

Related U.S. Application Data
(63) Continuation of application No. PCT/US2022/022997, filed on Apr. 1, 2022.
(60) Provisional application No. 63/170,422, filed on Apr. 2, 2021.

(51) Int. Cl. C07D 487/04; C12P 7/62; C07D 207/273

(52) U.S. Cl. CPC C07D 487/04; C12P 7/62; C07D 207/273

(57) ABSTRACT

Processes are provided herein for the preparation of a bicyclic ketone compound of formula (I), or a stereoisomer thereof:

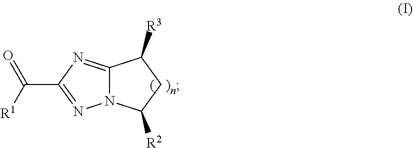

or a pharmaceutically acceptable salt thereof, wherein R¹, R², R³ and n are as defined herein; and compounds prepared by these processes.

### CROSS-REFERENCE TO RELATED APPLICATIONS

This application is a continuation of International Application No, PCT/US/2022/022997 filed Apr. 1, 2022 which claims the benefit of priority to U.S. Provisional Patent Application No. 63/170,422, filed on Apr. 2, 2021, the content of which is incorporated by reference herein in its entirety.

### FIELD OF INVENTION

Provided herein are processes of making bicyclic ketone compounds useful for therapy and/or prophylaxis in a mammal, in addition to compounds prepared by the processes. In particular, the bicyclic ketone compounds are chiral compounds of inhibitors of RIP1 kinase useful for treating diseases and disorders associated with inflammation, cell death and others.

### BACKGROUND

Receptor-interacting protein-1 (“RIP1”) kinase is a serine/threonine protein kinase. RIP1 is a regulator of cell signaling that is involved, among other things, in the mediation of programmed cell death pathways, e.g., necroptosis. The best studied form of necroptotic cell death is initiated by TNFα (tumor necrosis factor), but necroptosis can also be induced by other members of the TNFα death ligand family (Fas and TRAIL/Apo2L), interferons, Toll-like receptors (TLRs) signaling and viral infection via the DNA sensor DAI (DNA-dependent activator of interferon regulatory factor) [1-3]. Binding of TNFα to the TNFR1 (TNF receptor 1) prompts TNFR1 trimerization and formation of an intracellular complex, Complex-I. TRADD (TNF receptor associated death domain protein) binds to the intracellular death domain of TNFR1 and recruits the protein kinase RIP1 (receptor-interacting protein 1) through the death domain present in both proteins [4]. Following initial recruitment into TNFR1-associated signaling complex, RIP1 translocates to a secondary cytoplasmatic complex, Complex-II [5-7]. Complex-II is formed by the death domain containing protein FADD (Fas-associated Protein), RIP1, caspase-8 and cFLIP. If caspase-8 is not fully activated or its activity is blocked, the protein kinase RIP3 gets recruited to the complex, forming a necrosome, which will lead to necroptotic cell death initiation [8-10]. Once the necrosome is formed, RIP1 and RIP3 engage in a series of auto and cross phosphorylation events that are essential for necroptotic cell death. Necroptosis can be completely blocked either by the kinase inactivating mutation in any of the two kinases, or chemically by RIP1 kinase inhibitors (necrostatins), or RIP3 kinase inhibitors [11-13]. Phosphorylation of RIP3 allows the binding and phosphorylation of pseudokinase MLKL (mixed lineage kinase domain-like), a key component of necroptotic cell death [14, 15].

Necroptosis has crucial pathophysiological relevance in myocardial infarction, stroke, atherosclerosis, ischemia-reperfusion injury, inflammatory bowel diseases, retinal degeneration and a number of other common clinical disorders [16]. Therefore, selective inhibitors of RIP1 kinase activity are therefore desired as a potential treatment of diseases mediated by this pathway and associated with inflammation and/or necroptotic cell death.

Inhibitors of RIP1 kinase have been previously described. The first published inhibitor of RIP1 kinase activity was necrostatin 1 (Nec-1) [17]. This initial discovery was followed by modified versions of Nec-1 with various abilities to block RIP1 kinase activity [11, 18]. Recently, additional RIP1 kinase inhibitors have been described that differ structurally from necrostatin class of compounds [19, 20, 21].

Synthesis of the inhibitors, including, for example, particular stereoisomers, is difficult however. There is accordingly a need for new synthetic procedures for making the inhibitors.

References cited herein, each of which is hereby incorporated by reference in its entirety:
1) Vanden Berghe, T., Linkermann, A., Jouan-Lanhouet, S., Walczak, H. and Vandenabeele, P. (2014) Regulated necrosis: the expanding network of non-apoptotic cell death pathways. Nature reviews. Molecular cell biology. 15, 135-147.
2) Newton, K. (2015) RIPK1 and RIPK3: critical regulators of inflammation and cell death. Trends in cell biology. 25, 347-353.
3) de Almagro, M. C. and Vucic, D. (2015) Necroptosis: Pathway diversity and characteristics. Semin Cell Dev Biol. 39, 56-62.
4) Chen, Z. J. (2012) Ubiquitination in signaling to and activation of IKK. Immunological reviews. 246, 95-106.
5) O'Donnell, M. A., Legarda-Addison, D., Skountzos, P., Yeh, W. C. and Ting, A. T. (2007) Ubiquitination of RIP1 regulates an NF-kappaB-independent cell-death switch in TNF signaling. Curr Biol. 17, 418-424.
6) Feoktistova, M., Geserick, P., Kellert, B., Dimitrova, D. P., Langlais, C., Hupe, M., Cain, K., MacFarlane, M., Hacker, G. and Leverkus, M. (2011) cIAPs block Ripoptosome formation, a RIP1/caspase-8 containing intracellular cell death complex differentially regulated by cFLIP isoforms. Molecular cell. 43, 449-463.
7) Bertrand, M. J., Milutinovic, S., Dickson, K. M., Ho, W. C., Boudreault, A., Durkin, J., Gillard, J. W., Jaquith, J. B., Morris, S. J. and Barker, P. A. (2008) cIAP1 and cIAP2 facilitate cancer cell survival by functioning as E3 ligases that promote RIP1 ubiquitination. Mol Cell. 30, 689-700.
8) Wang, L., Du, F. and Wang, X. (2008) TNF-alpha induces two distinct caspase-8 activation pathways. Cell. 133, 693-703.
9) He, S., Wang, L., Miao, L., Wang, T., Du, F., Zhao, L. and Wang, X. (2009) Receptor interacting protein kinase-3 determines cellular necrotic response to TNF-alpha. Cell. 137, 1100-1111.
10) Cho, Y. S., Challa, S., Moquin, D., Genga, R., Ray, T. D., Guildford, M. and Chan, F. K. (2009) Phosphorylation-driven assembly of the RIP1-RIP3 complex regulates programmed necrosis and virus-induced inflammation. Cell. 137, 1112-1123.
11) Degterev, A., Hitomi, J., Germscheid, M., Ch'en, I. L., Korkina, O., Teng, X., Abbott, D., Cuny, G. D., Yuan, C., Wagner, G., Hedrick, S. M., Gerber, S. A., Lugovskoy, A. and Yuan, J. (2008) Identification of RIP1 kinase as a specific cellular target of necrostatins. Nat Chem Biol. 4, 313-321.
12) Newton, K., Dugger, D. L., Wickliffe, K. E., Kapoor, N., de Almagro, M. C., Vucic, D., Komuves, L., Ferrando, R. E., French, D. M., Webster, J., Roose-Girma, M., Warming, S. and Dixit, V. M. (2014) Activity of protein kinase RIPK3 determines whether cells die by necroptosis or apoptosis. Science. 343, 1357-1360.
13) Kaiser, W. J., Sridharan, H., Huang, C., Mandal, P., Upton, J. W., Gough, P. J., Sehon, C. A., Marquis, R. W., Bertin, J. and Mocarski, E. S. (2013) Toll-like receptor 3-mediated necrosis via TRIF, RIP3, and MLKL. The Journal of biological chemistry. 288, 31268-31279.
14) Zhao, J., Jitkaew, S., Cai, Z., Choksi, S., Li, Q., Luo, J. and Liu, Z. G. (2012) Mixed lineage kinase domain-like is a key receptor interacting protein 3 downstream component of TNF-induced necrosis. Proceedings of the National Academy of Sciences of the United States of America. 109, 5322-5327.
15) Sun, L., Wang, H., Wang, Z., He, S., Chen, S., Liao, D., Wang, L., Yan, J., Liu, W., Lei, X. and Wang, X. (2012) Mixed Lineage Kinase Domain-like Protein Mediates Necrosis Signaling Downstream of RIP3 Kinase. Cell. 148, 213-227.
16) Linkermann, A. and Green, D. R. (2014) Necroptosis. The New England journal of medicine. 370, 455-465.
17) Degterev, A., Huang, Z., Boyce, M., Li, Y., Jagtap, P., Mizushima, N., Cuny, G. D., Mitchison, T. J., Moskowitz, M. A. and Yuan, J. (2005) Chemical inhibitor of nonapoptotic cell death with therapeutic potential for ischemic brain injury. Nat Chem Biol. 1, 112-119.
18) Takahashi, N., Duprez, L., Grootjans, S., Cauwels, A., Nerinckx, W., DuHadaway, J. B., Goossens, V., Roelandt, R., Van Hauwermeiren, F., Libert, C., Declercq, W., Callewaert, N., Prendergast, G. C., Degterev, A., Yuan, J. and Vandenabeele, P. (2012) Necrostatin-1 analogues: critical issues on the specificity, activity and in vivo use in experimental disease models. Cell Death Dis. 3, e437.
19) Harris, P. A., Bandyopadhyay, D., Berger, S. B., Campobasso, N., Capriotti, C. A., Cox, J. A., Dare, L., Finger, J. N., Hoffman, S. J., Kahler, K. M., Lehr, R., Lich, J. D., Nagilla, R., Nolte, R. T., Ouellette, M. T., Pao, C. S., Schaeffer, M. C., Smallwood, A., Sun, H. H., Swift, B. A., Totoritis, R. D., Ward, P., Marquis, R. W., Bertin, J. and Gough, P. J. (2013) Discovery of Small Molecule RIP1 Kinase Inhibitors for the Treatment of Pathologies Associated with Necroptosis. ACS medicinal chemistry letters. 4, 1238-1243.
20) Najjar, M., Suebsuwong, C., Ray, S. S., Thapa, R. J., Maki, J. L., Nogusa, S., Shah, S., Saleh, D., Gough, P. J., Bertin, J., Yuan, J., Balachandran, S., Cuny, G. D. and Degterev, A. (2015) Structure Guided Design of Potent and Selective Ponatinib-Based Hybrid Inhibitors for RIPK1. Cell Rep.
21) International Patent Publication No. WO 2014/125444.
22) International Patent Publication No. WO 2017/004500.

### SUMMARY

Provided herein are solution to these problems and more.

In one aspect, processes are provided herein for the preparation of a chiral bicyclic ketone compound of formula (I) or formula (II):

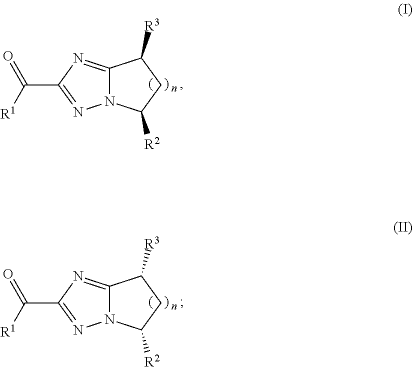

or a pharmaceutically acceptable salt thereof, wherein:
   R¹ is selected from the group consisting of C₁-C₆ alkyl, C₃-C₆ cycloalkyl, C₁-C₆ alkoxy, C₁-C₆ haloalkyl, C₁-C₆ haloalkoxy, C₁-C₆ alkyl-N(R^{N})₂, phenyl, benzyl, 4 to 8 membered heterocyclyl and 5 to 6 membered heteroaryl, wherein R¹ is bound to the adjacent carbonyl by a carbon atom and R¹ is optionally substituted by one or two substituents selected from the group consisting of F, Cl, Br, C₁-C₆ alkyl, C₃-C₆ cycloalkyl, C₁-C₆ alkoxy, C₁-C₆ haloalkyl, C₁-C₆ haloalkoxy, C₁-C₆ alkyl-N(R^{N})₂, hydroxyl, hydroxymethyl, cyano, cyanomethyl, cyanoethyl, C(O)C₁-C₆ alkyl, phenyl, benzyl, CH₂—(C₃-C₆ cycloalkyl), 5 to 6 membered heteroaryl, and CH₂-(5 to 6 membered heteroaryl);
   each R^{N} is independently selected from the group consisting of H, C₁-C₆ alkyl, C₃-C₆ cycloalkyl, C₁-C₆ alkoxy, and C₁-C₆ haloalkyl; or two R^{N} may together with the adjacent N form a 4-6 membered ring;
   R² is selected from the group consisting of H, C₁-C₆ alkyl, C₁-C₆ haloalkyl, C₃-C₆ cycloalkyl, C₁-C₆ alkoxy, C₁-C₆ haloalkoxy, C₁-C₆ thioalkyl, phenyl, benzyl, CH₂—(C₃-C₆ cycloalkyl), CH₂CH₂—(C₃-C₆ cycloalkyl), CH₂-(4 to 6 membered heterocyclyl), CH₂CH₂-(4 to 6 membered heterocyclyl), 5 to 6 membered heteroaryl, and CH₂-(5 to 6 membered heteroaryl); wherein when a phenyl ring is present it may be substituted by 1 to 3 substituents selected from the group consisting of halogen, C₁-C₄ alkyl, C₁-C₄ haloalkyl, C₁-C₄ alkoxy, C₁-C₄ haloalkoxy, and cyano;
   R³ is selected from the group consisting of D, halogen, OH, CN, C₁-C₄ alkyl, C₁-C₄ haloalkyl, cyclopropyl, C₁-C₄ alkoxy and C₁-C₄ haloalkoxy; and
   n is 1, 2 or 3.

In another aspect, a process is provided herein for the preparation of a chiral bicyclic ketone compound of formula (I), or a stereoisomer, or a pharmaceutically acceptable salt thereof, wherein R¹, R², R³ and n are as defined herein, the process comprising:
(a) contacting a compound of chiral N-amino lactam formula p, or a stereoisomer thereof:

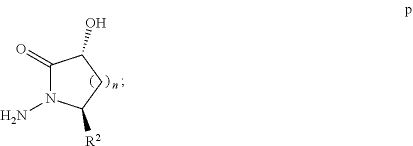

or a salt thereof, in the presence of an acid additive and an alcohol solvent with an imidate compound of formula c:

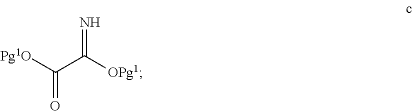

or a salt thereof, to form a chiral bicyclic triazole compound of formula x, or a stereoisomer thereof:

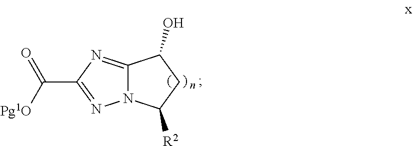

or a salt thereof; wherein:
   Pg¹ is an optional hydroxyl protecting group and may be the same or different on each occurrence; and
   the chiral bicyclic triazole compound of formula x, or the stereoisomer thereof, is an intermediate compound in the preparation of the chiral bicyclic ketone compound of formula (I), or the stereoisomer thereof.

In another aspect, a process is provided herein for the preparation of a chiral N-amino lactam compound of formula p, or a stereoisomer thereof:

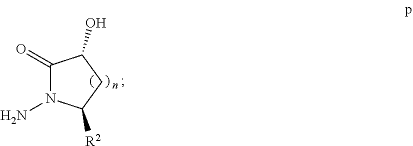

or a salt thereof, wherein R² and n are as defined herein, the process comprising:
   (a) reacting a chiral hydroxydicarboxylic acid compound of formula d or a stereoisomer thereof:

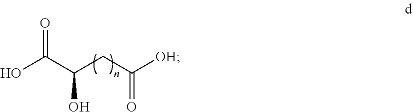

or a salt thereof, in the presence of an acid chloride of formula e:

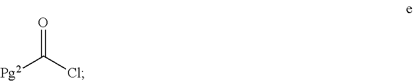

or a salt thereof, to form a chiral carboxylic cyclic anhydride compound of formula f or a stereoisomer thereof:

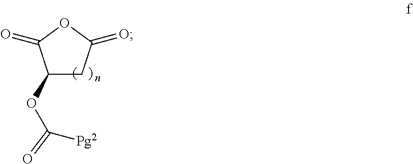

or a salt thereof; and
   (b) reacting a protected hydrazone compound of formula 1, or a stereoisomer thereof:

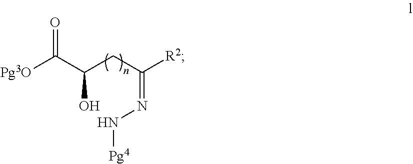

or a salt thereof, in the presence of an acid additive, to form a chiral hydroxy ester hydrazine compound of formula m or a stereoisomer thereof:

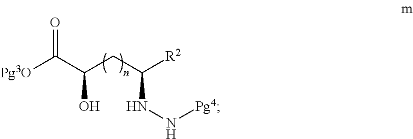

or a salt thereof; wherein:
   Pg² is optionally substituted C₁-C₆ alkyl, C₃-C₆ cycloalkyl or aryl;
   Pg³ is an optional hydroxyl protecting group and may be the same or different on each occurrence;
   Pg⁴ is an optional amine protecting group and may be the same or different on each occurrence;
   the chiral carboxylic cyclic anhydride compound of formula f, or the stereoisomer thereof, is an intermediate in the preparation of the protected hydrazone compound of formula 1, or the stereoisomer thereof; and
   the chiral hydroxy ester hydrazine compound of formula m, or the stereoisomer thereof, is an intermediate in the preparation of the chiral N-amino lactam compound of formula p, or the stereoisomer thereof.

In another aspect, a process is provided herein for the preparation of the chiral N-amino lactam compound of formula p, or a stereoisomer thereof, or a salt thereof, the process comprising:
(a) reacting a chiral hydroxydicarboxylic acid compound of formula d or a stereoisomer thereof:

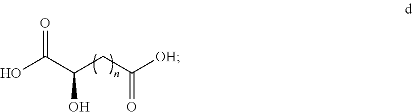

or a salt thereof, in the presence of an acid chloride of formula e:

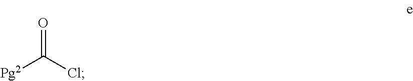

or a salt thereof, to form a chiral carboxylic cyclic anhydride compound of formula f or a stereoisomer thereof:

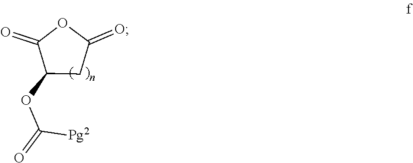

or a salt thereof; wherein:
   Pg², Pg³ and Pg⁴ are as defined herein; and
   the chiral carboxylic cyclic anhydride compound of formula f, or the stereoisomer thereof, is an intermediate in the preparation of the chiral N-amino lactam compound of formula p, or the stereoisomer thereof.

In another aspect, a process is provided herein for the preparation of the chiral N-amino lactam compound of formula p, or a stereoisomer thereof, or a salt thereof, the process comprising:
(b) reacting a protected hydrazone compound of formula 1, or a stereoisomer thereof:

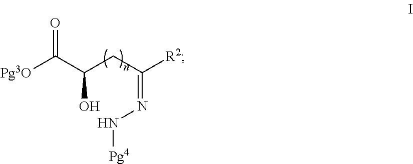

or a salt thereof, in the presence of an acid additive, to form a chiral hydroxy ester hydrazine compound of formula m, or a stereoisomer thereof:

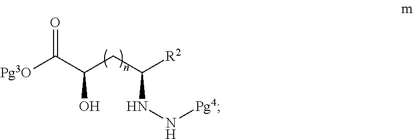

or a salt thereof; wherein:
   Pg², Pg³ and Pg⁴ are as defined herein; and
   the chiral hydroxy ester hydrazine compound of formula m, or the stereoisomer thereof, is an intermediate in the preparation of the chiral N-amino lactam compound of formula p, or the stereoisomer thereof.

In another aspect, a process is provided herein for the preparation of an imidate salt compound of formula b:

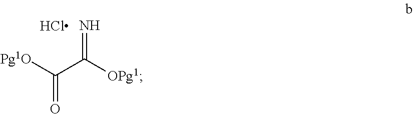

the process comprising:
(a) reacting a cyanoformate compound of formula a:

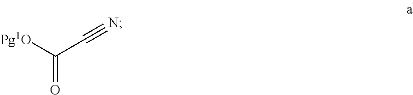

in the presence of an anhydrous acid source in an alcohol solvent to form the imidate salt compound of formula b, wherein the anhydrous acid source is TMSCl, the acid is HCl, and Pg¹ is an optional hydroxyl protecting group and may be the same or different on each occurrence.

In another aspect, processes are provided herein for the preparation of a chiral bicyclic ketone compound of formula (III) or formula (IV):

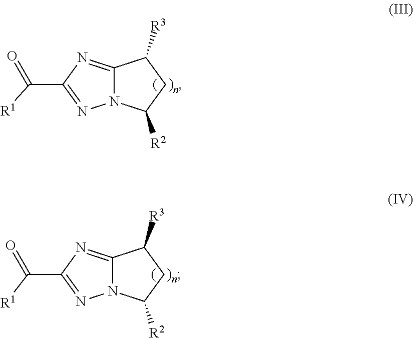

or a pharmaceutically acceptable salt thereof, wherein R¹, R², R³ and n are as defined herein.

In another aspect, a process is provided herein for the preparation of a hydroxyketoester compound of formula j, or a stereoisomer thereof:

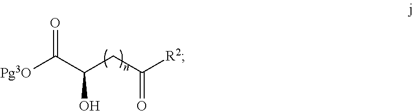

the process comprising:
(a) reacting a diketoester compound of formula hh:

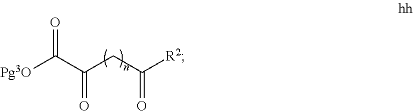

in the presence of a ketoreductase to form the hydroxyketoester compound of formula j, or a stereoisomer thereof, or salt thereof, wherein R², Pg³ and n are as defined herein.

In another aspect, compounds are provided herein prepared by the processes described herein.

The subject methods provide unexpectedly better overall yield in producing compounds of formula (I), formula (II), formula (III) or formula (IV), or pharmaceutically acceptable salts thereof, as well as improved product purity, improved diastereomeric ratio, improved stereomeric excess, via avoidance of potential racemization events, and improved yield. Additional embodiments and details are provided below.

### DETAILED DESCRIPTION

### Definitions

As provided herein, all chemical formulae and generic chemical structures should be interpreted to provide proper valence and chemically stable bonds between atoms as understood by one of ordinary skill in the art. Where appropriate, substituents may be bonded to more than one adjacent atom (e.g., alkyl includes methylene where two bonds are present).

In the chemical formulae provided herein, “halogen” or “halo” refers to fluorine, chlorine, and bromine (i.e., F, Cl, Br).

“Alkyl”, unless otherwise specifically defined, refers to an optionally substituted, straight-chain or branched C₁-C₁₂ alkyl group. In some embodiments, “alkyl” refers to a C₁-C₆ alkyl group. Exemplary alkyl groups include methyl, ethyl, propyl, iso-propyl, n-butyl, iso-butyl, tert-butyl, sec-butyl, n-pentyl, n-hexyl, n-heptyl, and n-octyl. Substituted alkyl groups provided herein are substituted by one or more substituents selected from the group consisting of halogen, cyano, trifluoromethyl, methoxy, ethoxy, difluoromethoxy, trifluoromethoxy, C₃-C₆ cycloalkyl, phenyl, OH, CO₂H, CO₂(C₁-C₄ alkyl), NH₂, NH(C₁-C₄ alkyl), N(C₁-C₄ alkyl)₂, NH(C═O)C₁-C₄ alkyl, (C═O)NH(C₁-C₄ alkyl), (C═O)N(C₁-C₄ alkyl)₂, S(C₁-C₄ alkyl), SO(C₁-C₄ alkyl), SO₂(C₁-C₄ alkyl), SO₂NH(C₁-C₄ alkyl), SO₂N(C₁-C₄ alkyl)₂, and NHSO₂(C₁-C₄ alkyl). In some embodiments, the substituted alkyl group has 1 or 2 substituents. In some embodiments, the alkyl group is unsubstituted.

“Cycloalkyl”, unless otherwise specifically defined, refers to an optionally substituted C₃-C₁₂ cycloalkyl group and includes fused, spirocyclic, and bridged bicyclic groups, wherein the substituents are selected from the group consisting of halogen, cyano, trifluoromethyl, methoxy, ethoxy, difluoromethoxy, trifluoromethoxy, C₃-C₆ cycloalkyl, phenyl, OH, CO₂H, CO₂(C₁-C₄ alkyl), NH₂, NH(C₁-C₄ alkyl), N(C₁-C₄ alkyl)₂, NH(C═O)C₁-C₄ alkyl, (C═O)NH(C₁-C₄ alkyl), (C═O)N(C₁-C₄ alkyl)₂, S(C₁-C₄ alkyl), SO(C₁-C₄ alkyl), SO₂(C₁-C₄ alkyl), SO₂NH(C₁-C₄ alkyl), SO₂N(C₁-C₄ alkyl)₂, and NHSO₂(C₁-C₄ alkyl). In some embodiments, cycloalkyl refers to a C₃-C₆ cycloalkyl group. In some embodiments, the C₃-C₆ cycloalkyl group is optionally substituted with 1 to three halogen atoms. In some embodiments, the C₃-C₆ cycloalkyl group is optionally substituted with 1 to three fluorine atoms. Exemplary C₃-C₆ cycloalkyl groups include cyclopropyl, cyclobutyl, cyclopentyl and cyclohexyl. Exemplary C₃-C₁₂ cycloalkyl groups further include bicyclo[3.1.0]hexyl, bicyclo[2.1.1]hexyl, cycloheptyl, bicycle[4.1.0]heptyl, spiro[4.2]heptyl, cyclooctyl, spiro[4.3]octyl, spiro[5.2]octyl, bicyclo[2.2.1]heptanyl, bicycle[2.2.2]octanyl, adamantanyl, decalinyl, and spiro[5.4]decanyl. Where appropriate, cycloalkyl groups may be fused to other groups such that more than one chemical bond exists between the cycloalkyl group and another ring system (e.g., the C ring of formula I). In some embodiments, the cycloalkyl group is unsubstituted.

“Haloalkyl”, unless otherwise specifically defined, refers to a straight-chain or branched C₁-C₁₂ alkyl group, wherein one or more hydrogen atoms are replaced by a halogen. In some embodiments, “haloalkyl” refers to a C₁-C₆ haloalkyl group. In some embodiments, 1 to 3 hydrogen atoms of the haloalkyl group are replaced by a halogen. In some embodiments, every hydrogen atom of the haloalkyl group is replaced by a halogen (e.g, trifluoromethyl). In some embodiments, the haloalkyl is as defined herein wherein the halogen in each instance is fluorine. Exemplary haloalkyl groups include fluoromethyl, difluoromethyl, trifluoromethyl, trifluoroethyl, and pentafluoroethyl.

“Alkoxy”, unless otherwise specifically defined, refers to a straight-chain or branched C₁-C₁₂ alkyl group, wherein one or more oxygen atoms are present, in each instance between two carbon atoms. In some embodiments, “alkoxy” refers to a C₁-C₆ alkoxy group. In some embodiments, C₁-C₆ alkoxy groups provided herein have one oxygen atom. Exemplary alkoxy groups include methoxy, ethoxy, CH₂OCH₃, CH₂CH₂OCH₃, CH₂OCH₂CH₃, CH₂CH₂OCH₂CH₃, CH₂OCH₂CH₂CH₃, CH₂CH₂CH₂OCH₃, CH₂OCH(CH₃)₂, CH₂OC(CH₃)₃, CH(CH₃)OCH₃, CH₂CH(CH₃)OCH₃, CH(CH₃)OCH₂CH₃, CH₂OCH₂OCH₃, CH₂CH₂OCH₂CH₂OCH₃, and CH₂OCH₂OCH₂OCH₃.

“Cycloalkoxy”, unless otherwise specifically defined, refers to a C₄-C₁₀ or a C₄-C₆ alkoxy group as defined above wherein the group is cyclic and contains one oxygen atom. Exemplary cycloalkoxy groups include oxetanyl, tetrahydrofuranyl, and tetrahydropyranyl.

“Haloalkoxy”, unless otherwise specifically defined, refers to a C₁-C₆ haloalkyl group as defined above, wherein one or two oxygen atoms are present, in each instance between two carbon atoms. In some embodiments, C₁-C₆ haloalkoxy groups provided herein have one oxygen atom. Exemplary haloalkoxy groups include OCF₃, OCHF₂ and CH₂OCF₃.

“Thioalkyl”, unless otherwise specifically defined, refers to a C₁-C₁₂ or a C₁-C₆ alkoxy group as defined above wherein the oxygen atom is replaced by a sulfur atom. In some embodiments, thioalkyl groups may include sulfur atoms substituted by one or two oxygen atoms (i.e., alkylsulfones and alkylsulfoxides). Exemplary thioalkyl groups are those exemplified in the definition of alkoxy above, wherein each oxygen atom is replaced by a sulfur atom in each instance.

“Thiocycloalkyl”, unless otherwise specifically defined, refers to a C₄-C₁₀ or a C₄-C₆ thioalkyl group as defined above wherein the group is cyclic and contains one sulfur atom. In some embodiments, the sulfur atom of the thiocycloalkyl group is substituted by one or two oxygen atoms (i.e., a cyclic sulfone or sulfoxide). Exemplary thiocycloalkyl groups include thietanyl, thiolanyl, thianyl, 1,1-dioxothiolanyl, and 1,1-dioxothianyl.

“Heterocyclyl”, unless otherwise specifically defined, refers to a single saturated or partially unsaturated 4 to 8 membered ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems have from 7 to 12 atoms and are further described below. Thus, the term includes single saturated or partially unsaturated rings (e.g., 3, 4, 5, 6, 7 or 8 membered rings) from about 1 to 7 carbon atoms and from about 1 to 4 heteroatoms selected from the group consisting of oxygen, nitrogen and sulfur in the ring. The ring may be C-branched (i.e., substituted by C₁-C₄ alkyl). The ring may be substituted with one or more (e.g., 1, 2 or 3) oxo groups and the sulfur and nitrogen atoms may also be present in their oxidized forms. Exemplary heterocycles include but are not limited to azetidinyl, tetrahydrofuranyl and piperidinyl. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system. It is also to be understood that the point of attachment for a heterocycle or heterocycle multiple condensed ring system can be at any suitable atom of the heterocyclyl group including a carbon atom and a nitrogen atom. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one N-methylpiperidine, imidazolidine, pyrazolidine, butyrolactam, valerolactam, imidazolidinone, hydantoin, dioxolane, phthalimide, 1,4-dioxane, thiomorpholine, thiomorpholine-S-oxide, thiomorpholine-S,S-oxide, pyran, 3-pyrroline, thiopyran, pyrone, tetrhydrothiophene, quinuclidine, tropane, 2-azaspiro[3.3]heptane, (1R,5S)-3-azabicyclo[3.2.1]octane, (1s,4s)-2-azabicyclo[2.2.2]octane, (1R,4R)-2-oxa-5-azabicyclo[2.2.2]octane and pyrrolidin-2-one.

In some embodiments, the heterocyclyl is a C₄-C₁₀ heterocyclyl having 1 to 3 heteroatoms selected from the group consisting of nitrogen, oxygen and sulfur. In some embodiments, the heterocyclyl group is neither bicyclic nor spirocyclic. In some embodiments, the heterocyclyl is a C₅-C₆ heterocylcyl having 1 to 3 heteroatoms, wherein at least 2 are nitrogen if 3 heteroatoms are present.

“Aryl”, unless otherwise specifically defined, refers to a single all carbon aromatic ring or a multiple condensed all carbon ring system wherein at least one of the rings is aromatic and wherein the aryl group has 6 to 20 carbon atoms, 6 to 14 carbon atoms, 6 to 12 carbon atoms, or 6 to 10 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Exemplary aryl groups include phenyl, indenyl, naphthyl, 1, 2, 3, 4-tetrahydronaphthyl, anthracenyl, and the like.

“Heteroaryl”, unless otherwise specifically defined, refers to a 5 to 6 membered aromatic ring that has at least one atom other than carbon in the ring, wherein the atom is selected from the group consisting of oxygen, nitrogen and sulfur; “heteroaryl” also includes multiple condensed ring systems having 8 to 16 atoms that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2 or 3 rings) wherein a heteroaryl group, as defined above, is condensed with one or more rings selected from heteroaryls (to form for example a naphthyridinyl such as 1,8-naphthyridinyl), heterocycles, (to form for example a 1, 2, 3, 4-tetrahydronaphthyridinyl such as 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has 1 to 15 carbon atoms and about 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system. It is also to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b,4,4a,5-tetrahydro-1H-cyclopropa[3,4]cyclo-penta[1,2-c]pyrazole.

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space.

As used herein a wavy line “ [symbol] ” that intersects a bond in a chemical structure indicates the point of attachment of the bond that the wavy bond intersects in the chemical structure to the remainder of a molecule.

As used herein, the term “C-linked” means that the group that the term describes is attached the remainder of the molecule through a ring carbon atom.

As used herein, the term “N-linked” means that the group that the term describes is attached to the remainder of the molecule through a ring nitrogen atom.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds,” John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 97% the absolute stereoisomer depicted. In another embodiment, the compound may be at least 98% the absolute stereoisomer depicted. In another embodiment, the compound may be at least 99% the absolute stereoisomer depicted.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water. In some embodiments, a hydrate of a compound provided herein is a ketone hydrate.

As used herein, the term “protective group” or “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein, the term “mammal” includes, but is not limited to, humans, mice, rats, guinea pigs, monkeys, dogs, cats, horses, cows, pigs, and sheep.

As used herein, the term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. As used herein the term “prodrug” refers to those compounds that readily undergo chemical changes under physiological conditions to provide the compounds of the present invention.

Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Prodrugs of the invention include compounds wherein an amino acid residue, or a polypeptide chain of two or more (e.g., two, three or four) amino acid residues, is covalently joined through an amide or ester bond to a free amino, hydroxy or carboxylic acid group of a compound of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, citrulline, homocysteine, homoserine, methyl-alanine, para-benzoylphenylalanine, phenylglycine, propargylglycine, sarcosine, methionine sulfone and tert-butylglycine.

Additional types of prodrugs are also encompassed. For instance, a free carboxyl group of a compound of the invention can be derivatized as an amide or alkyl ester. As another example, compounds of this invention comprising free hydroxy groups can be derivatized as prodrugs by converting the hydroxy group into a group such as, but not limited to, a phosphate ester, hemisuccinate, dimethylaminoacetate, or phosphoryloxymethyloxycarbonyl group, as outlined in Fleisher, D. et al., (1996) Improved oral drug delivery: solubility limitations overcome by the use of prodrugs Advanced Drug Delivery Reviews, 19:115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group can be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl, (C₁₋₆)alkoxycarbonyloxymethyl, N—(C₁₋₆)alkoxycarbonylaminomethyl, succinoyl, (C₁₋₆)alkanoyl, alpha-amino(C₁₋₄)alkanoyl, arylacyl and alpha-aminoacyl, or alpha-aminoacyl-alpha-aminoacyl, where each alpha-aminoacyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

Additionally, the present invention provides for metabolites of compounds of the invention. As used herein, a “metabolite” refers to a product produced through metabolism in the body of a specified compound or salt thereof. Such products can result for example from the oxidation, reduction, hydrolysis, amidation, deamidation, esterification, deesterification, enzymatic cleavage, and the like, of the administered compound.

Metabolite products typically are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention can exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention.

The term “composition”, as used herein, is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “treat” and “treatment” refer to both therapeutic treatment and/or prophylactic treatment or preventative measures, wherein the object is to prevent or slow down (lessen) an undesired physiological change or disorder, such as, for example, the development or spread of cancer. For purposes of this invention, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treatment” can also mean prolonging survival as compared to expected survival if not receiving treatment. Those in need of treatment include those already with the disease or disorder as well as those prone to have the disease or disorder or those in which the disease or disorder is to be prevented.

The phrase “therapeutically effective amount” or “effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. For cancer therapy, efficacy can, for example, be measured by assessing the time to disease progression (TTP) and/or determining the response rate (RR).

The term “bioavailability” refers to the systemic availability (i.e., blood/plasma levels) of a given amount of drug administered to a patient. Bioavailability is an absolute term that indicates measurement of both the time (rate) and total amount (extent) of drug that reaches the general circulation from an administered dosage form.

“Ketoreductase” and “KRED” are used interchangeably herein to refer to a polypeptide having an enzymatic capability of reducing a carbonyl group to its corresponding alcohol. For example, certain enzymes belonging to the ketoreductase (KRED) or carbonyl reductase class (EC 1.1.1.184) have been found to be useful for the stereoselective of pro-stereoisomeric aldehyde or ketone substrates to the corresponding chiral alcohol products. KREDs typically convert a ketone or aldehyde substrate to the corresponding alcohol product, but may also catalyze the reverse reaction, oxidation of an alcohol substrate to the corresponding ketone/aldehyde product, referred then sometimes to, for example, as alcohol dehydrogenases (ADHs). Alcohol dehydrogenases (EC 1.1.1.1) belong to a group of enzymes that facilitate the conversion between alcohols and aldehydes or ketones. The reduction of ketones and aldehydes and the oxidation of alcohols by enzymes such as KRED requires a cofactor, most commonly reduced nicotinamide adenine dinucleotide (NADH) or reduced nicotinamide adenine dinucleotide phosphate (NADPH), and nicotinamide adenine dinucleotide (NAD) or nicotinamide adenine dinucleotide phosphate (NADP) for the oxidation reaction. NADH and NADPH serve as electron donors, while NAD and NADP serve as electron acceptors. KREDs are increasingly being used for the stereoselective conversion of ketones and aldehydes to chiral alcohols compounds used in the production of key pharmaceutical compounds.

Examples using KREDs to generate useful chemical compounds include asymmetric reduction of 4-chloroacetoacetate esters (e.g., Zhou et al., J. Am. Chem. Soc. (1983), 105(18):5925-5926. Santaniello et al. J Chem Res., Synop. (1984). 4:132-133; U.S. Pat. Nos. 5,559,030; 5,700,670 and 5,891,685), reduction of dioxocatboxylic acids (e.g., U.S. Pat. No. 6,399,339), reduction of tert-butyl (S)-chloro-5-hydroxy-3-oxohexanoate (e.g., U.S. Pat. No. 6,645,746 and Int'l Pat. Pub. No. WO 01/40450), reduction ofpyrrolotriazine-based compounds (e.g., U.S. Pat. App. Pub. No. 2006/0286646), reduction of substituted acetophenones (e.g., U.S. Pat. No. 6,800,477), and reduction of ketothiolase (e g. Int'l Pat. Pub. No. WO 2005/054491 and Noey et al., Proc Natl. Acad. Sci. USA (2015), 112(15):E7065-E7072).

In some embodiments, the ketoreduction can be carried out in the presence of an alcohol, such as isopropanol, to provide a substrate for the reverse, oxidative reaction (alcohol dehydrogenation). In some embodiments, the NADH/NADPH consumed in the ketoreduction reaction is regenerated by the reverse, oxidative reaction. In some embodiments, the KREDs are capable of stereoselectively reducing ethyl 2,4-dioxo-4-phenyl-butanoate to the corresponding alcohol, (−)-ethyl (R)-2-hydroxy-4-oxo-4-phenylbutyrate. In some embodiments, the KREDs utilize a cofactor reduced nicotinamide adenine dinucleotide (NADH) or reduced nicotinamide adenine dinucleotide phosphate (NADPH) as the reducing agent. In other embodiments, the NADH/NADPH consumed in the ketoreduction reaction is regenerated by a coenzyme, such as glucose dehydrogenase or formate dehydrogenase. For example, the substrate of the glucose dehydrogenase is glucose, whereas formate is the substrate of the formate dehydrogenase. Ketoreductases and coenzymes as used herein include naturally occurring (wild type) as well as engineered ketoreductases and coenzymes. Examples of engineered ketoreductases are described, for example, in U.S. Pat. No. 7,820,421.

In some embodiments, when the process is carried out using whole cells of the host organism, the whole cell natively or recombinantly provides the KRED, the coenzyme, and/or the cofactor. In some embodiments, the engineered ketoreductase and/or coenzyme is added to the reaction mixture in the form of the purified enzyme, whole cells transformed with gene(s) encoding the enzymes, and/or cell extracts and/or lysates of such cells. The gene(s) encoding the engineered ketoreductase and/or coenzyme can be transformed into host cells separately or together into the same host cell. For example, one set of host cells can be transformed with gene(s) encoding the engineered ketoreductase and another set can be transformed with gene(s) encoding the coenzyme. Both sets of transformed cells can be utilized together in the reaction mixture in the form of whole cells, or in the form of lysates or extracts derived therefrom. In other embodiments, a host cell can be transformed with gene(s) encoding both the engineered ketoreductase and the coenzyme.

In some embodiments, whole cells transformed with gene(s) encoding the engineered ketoreductase or the coenzyme, or cell extracts and/or lysates thereof, are employed in a variety of different forms, including solid (e.g., lyophilized, spray-dried, and the like) or semisolid (e g, a crude paste). For example, the cell extracts or cell lysates may be partially purified by precipitation (ammonium sulfate, polyethyleneimine, heat treatment or the like, followed by a desalting procedure prior to lyophilization (e.g., ultrafiltration, dialysis, and the like). Any of the cell preparations may be stabilized by crosslinking using known crosslinking agents, such as, for example, glutaraldehyde or immobilization to a solid phase (e.g., Eupergit C, and the like).

In some embodiments, the present enzymes are used in various forms including a purified enzyme, a crude enzyme, a microbial culture, a bacterial cell, and a treated object thereof. Examples of the treated object used herein include a lyophilized bacterial cell, an acetone-dried bacterial cell, a ground bacterial cell, an autodigested substance of bacterial cell, an ultrasonic-treated object of bacterial cell, bacterial cell extract, or an alkaline-treated object of bacterial cell. In other embodiments, enzymes in various purities or forms as described above may be immobilized for use, for example, by known methods including an adsorption method to an inorganic carrier such as silica gel and ceramics, cellulose, ion-exchange resin and so on, a polyacrylamide method, a sulfur-containing polysaccharide gel method (for example, a carrageenan gel method), an alginic acid gel method, an agar gel method and so on. Any means of immobilizing enzymes generally known in the art may be used to immobilize the enzymes to a carrier. For example, the enzyme may be bound directly to a membrane, granules or the like of a resin having one or more functional groups, or it may be bound to the resin through bridging compounds having one or more functional groups, e.g. glutaraldehyde. Such enzyme immobilizing reactions are described, for example, on pages 369-394 of the 2nd Edition of Microbial Enzymes and Biotechnology (Elsevier Applied Science 1990; Ed. W. M. Fogarty and C. T. Kelly).

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

“Engineered ketoreductase” as used herein refers to a ketoreductase having a variant sequence generated by human manipulation (e.g., a sequence generated by directed evolution of a naturally occurring parent enzyme or directed evolution of a variant previously derived from a naturally occurring enzyme).

“Highly stereoselective” as used herein refers to a ketoreductase that is capable of converting or reducing a substrate to the corresponding product (e.g., ethyl 2,4-dioxo-4-phenyl-butanoate to (−)-ethyl (R)-2-hydroxy-4-oxo-4-phenylbutyrate) with at least about 99% stereomeric excess. In some embodiments, the KREDs are highly stereoselective. In some embodiments, “stereomeric excess” as used herein refers to enantiomeric excess. “Enantiomeric excess” or “ee” are used interchangeably herein to refer to the degree to which a sample contains one enantiomer compared to its corresponding non-superimposable mirror compound. A racemic mixture has an cc of 0%, whereas a sample including only one enantiomer has an ee of 100%.

### Methods of Making Inhibitors of Rip1 Kinase

Provided herein are processes for the preparation of compounds useful in the treatment of diseases and disorders associated with inflammation, cell death, neurological disorders and other diseases. In some embodiments, the prepared compounds includes inhibitors of RIP1 kinase useful in the treatment of such diseases and disorders. In some embodiments, the prepared compounds include compounds that are exemplified, for example, in U.S. Patent App. Publication US2019/0100530, the content of which is incorporated herein in its entirety. The processes described herein, for example, improve product purity, diastereomeric ratio (dr), stereomeric excess, and/or yield of the final products as well as key intermediates in the synthesis thereof. The processes described herein will be more fully understood with reference to the several reaction schemes below. In some embodiments, the processes unexpectedly provide improved product purity, improved diastereomeric ratio, improved stereomeric excess, and/or improved yield. Improved product purity includes, for example, improved chiral purity of the reaction product.

In some embodiments, processes are provided herein for the preparation of a compound of formula (I) or formula (II):

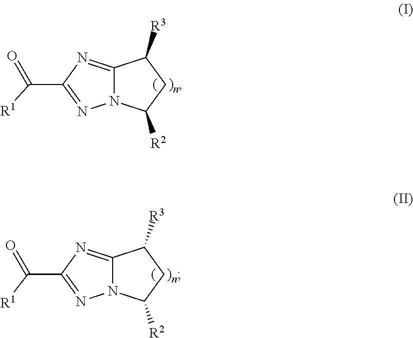

or pharmaceutically acceptable salts thereof, wherein:
   R¹ is selected from the group consisting of C₁-C₆ alkyl, C₃-C₆ cycloalkyl, C₁-C₆ alkoxy, C₁-C₆ haloalkyl, C₁-C₆ haloalkoxy, C₁-C₆ alkyl-N(R^{N})₂, phenyl, benzyl, 4 to 8 membered heterocyclyl and 5 to 6 membered heteroaryl, wherein R¹ is bound to the adjacent carbonyl by a carbon atom and R¹ is optionally substituted by one or two substituents selected from the group consisting of F, Cl, Br, C₁-C₆ alkyl, C₃-C₆ cycloalkyl, C₁-C₆ alkoxy, C₁-C₆ haloalkyl, C₁-C₆ haloalkoxy, C₁-C₆ alkyl-N(R^{N})₂, hydroxyl, hydroxymethyl, cyano, cyanomethyl, cyanoethyl, C(O)C₁-C₆ alkyl, phenyl, benzyl, CH₂—(C₃-C₆ cycloalkyl), 5 to 6 membered heteroaryl, and CH₂-(5 to 6 membered heteroaryl);
   each R^{N} is independently selected from the group consisting of H, C₁-C₆ alkyl, C₃-C₆ cycloalkyl, C₁-C₆ alkoxy, and C₁-C₆ haloalkyl; or two R^{N} may together with the adjacent N form a 4-6 membered ring;
   R² is selected from the group consisting of H, C₁-C₆ alkyl, C₁-C₆ haloalkyl, C₃-C₆ cycloalkyl, C₁-C₆ alkoxy, C₁-C₆ haloalkoxy, C₁-C₆ thioalkyl, phenyl, benzyl, CH₂—(C₃-C₆ cycloalkyl), CH₂CH₂—(C₃-C₆ cycloalkyl), CH₂-(4 to 6 membered heterocyclyl), CH₂CH₂-(4 to 6 membered heterocyclyl), 5 to 6 membered heteroaryl, and CH₂-(5 to 6 membered heteroaryl); wherein when a phenyl ring is present it may be substituted by 1 to 3 substituents selected from the group consisting of halogen, C₁-C₄ alkyl, C₁-C₄ haloalkyl, C₁-C₄ alkoxy, C₁-C₄ haloalkoxy, and cyano;
   R³ is selected from the group consisting of D, halogen, OH, CN, C₁-C₄ alkyl, C₁-C₄ haloalkyl, cyclopropyl, C₁-C₄ alkoxy and C₁-C₄ haloalkoxy; and
   n is 1, 2 or 3.

In some embodiments, a process is provided herein for the preparation of a chiral bicyclic ketone compound of formula (I), or a stereoisomer, or a pharmaceutically acceptable salt thereof, wherein R¹, R², R³ and n are as defined herein, the process comprising:
(a) contacting a compound of chiral N-amino lactam formula p, or a stereoisomer thereof:

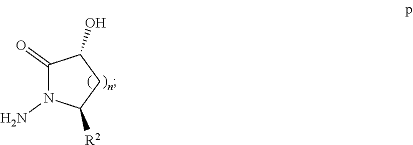

or a salt thereof, in the presence of an acid additive and an alcohol solvent with an imidate compound of formula c:

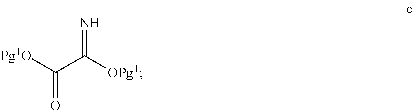

or a salt thereof, to form a chiral bicyclic triazole compound of formula x, or a stereoisomer thereof:

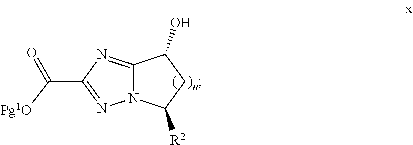

or a salt thereof; wherein:
   Pg¹ is an optional hydroxyl protecting group and may be the same or different on each occurrence; and
the chiral bicyclic triazole compound of formula x, or the stereoisomer thereof, is an intermediate compound in the preparation of the chiral bicyclic ketone compound of formula (I), or the stereoisomer thereof.

In some embodiments, a process for the preparation of a chiral N-amino lactam compound of formula p, or a stereoisomer thereof:

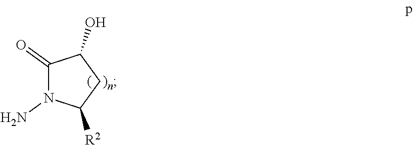

or a salt thereof, wherein R² and n are as defined herein, the process comprising:
   (a) reacting a chiral hydroxydicarboxylic acid compound of formula d or a stereoisomer

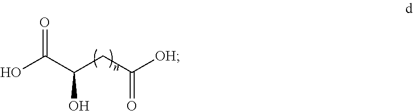

or a salt thereof, in the presence of organic acid chloride of formula e:

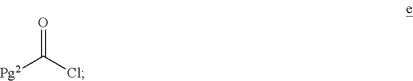

or a salt thereof, to form a chiral carboxylic cyclic anhydride compound of formula f or a stereoisomer thereof:

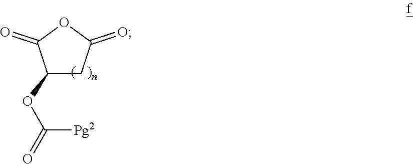

or a salt thereof; and
   (b) reacting a protected hydrazone compound of formula 1, or a stereoisomer thereof:

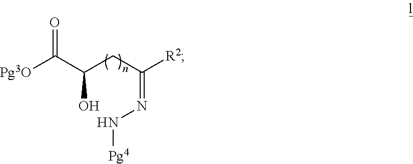

or a salt thereof, in the presence of an acid additive, to form a chiral hydroxy ester hydrazine compound of formula m or a stereoisomer thereof:

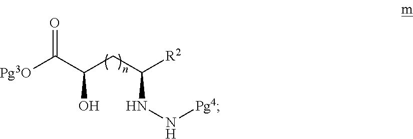

or a salt thereof; wherein:
   Pg² is optionally substituted C₁-C₆ alkyl, C₃-C₆ cycloalkyl or aryl;
   Pg³ is an optional hydroxyl protecting group and may be the same or different on each occurrence;
   Pg⁴ is an optional amine protecting group and may be the same or different on each occurrence;
   the chiral carboxylic cyclic anhydride compound of formula f, or the stereoisomer thereof, is an intermediate in the preparation of the protected hydrazone compound of formula 1, or the stereoisomer thereof; and
   the chiral hydroxy ester hydrazine compound of formula m or the stereoisomer thereof, is an intermediate in the preparation of the chiral N-amino lactam compound of formula p, or the stereoisomer thereof.

In some embodiments, a process for the preparation of the chiral N-amino lactam compound of formula p, or a stereoisomer thereof, or a salt thereof, the process comprising:
(a) reacting a chiral hydroxydicarboxylic acid compound of formula d or a stereoisomer thereof:

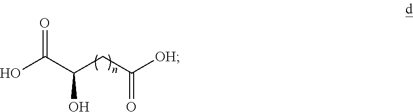

or a salt thereof, in the presence of organic acid chloride of formula e:

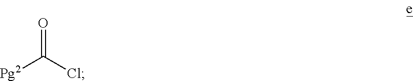

or a salt thereof, to form a chiral carboxylic cyclic anhydride compound of formula f or a stereoisomer thereof:

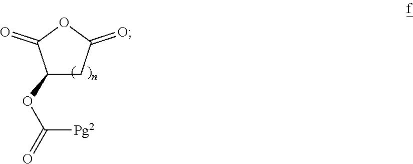

or a salt thereof; wherein:
   Pg², Pg³ and Pg⁴ are as defined herein; and
   the chiral carboxylic cyclic anhydride compound of formula f, or the stereoisomer thereof, is an intermediate in the preparation of the chiral N-amino lactam compound of formula p, or the stereoisomer thereof.

In some embodiments, a process for the preparation of the chiral N-amino lactam compound of formula p, or a stereoisomer thereof, or a salt thereof, the process comprising:
(b) reacting a protected hydrazone compound of formula 1, or a stereoisomer thereof:

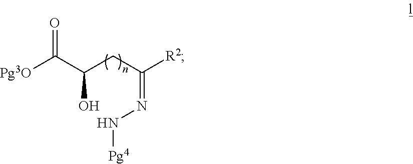

or a salt thereof, in the presence of an acid additive, to form a chiral hydroxy ester hydrazine compound of formula m or a stereoisomer thereof:

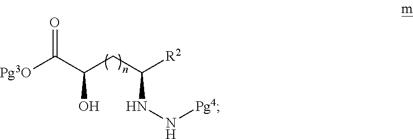

or a salt thereof; wherein:
   Pg², Pg³ and Pg⁴ are as defined herein; and
   the chiral hydroxy ester hydrazine compound of formula m or the stereoisomer thereof, is an intermediate in the preparation of the chiral N-amino lactam compound of formula p, or the stereoisomer thereof.

In some embodiments, a process for the preparation of an imidate salt compound of formula b:

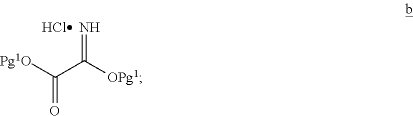

the process comprising:
(a) reacting a cyanoformate compound of formula a:

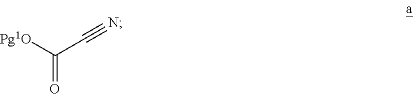

in the presence of an anhydrous acid source in an alcohol solvent to form the imidate salt compound of formula b, wherein the anhydrous acid source is TMSCl, the acid is HCl, and Pg¹ is an optional hydroxyl protecting group and may be the same or different on each occurrence.

In some embodiments, processes are provided herein for the preparation of a compound selected from the group consisting of:

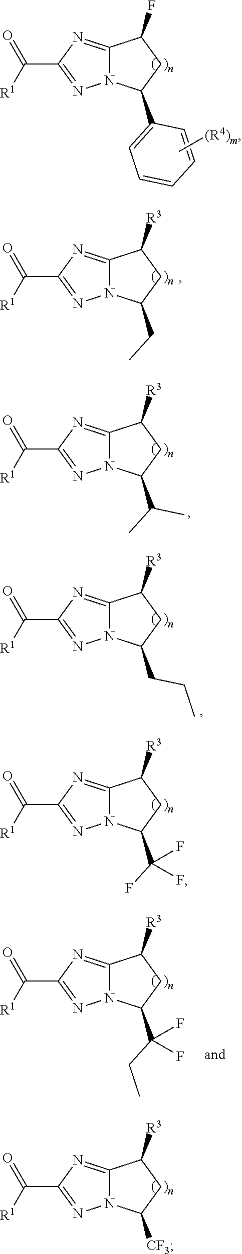

or a pharmaceutically acceptable salt thereof, wherein:
   R¹, R³ and n are as defined herein;
   each R⁴ is selected from the group consisting of H, F, Cl, C₁-C₆ alkyl, C₁-C₆ haloalkyl, C₁-C₆ alkoxy and C₁-C₆ haloalkoxy; and
   m is 0, 1, 2 or 3.

In some embodiments, processes are provided herein for the preparation of a compound that is:

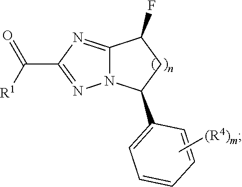

or a pharmaceutically acceptable salt thereof, wherein R¹, R⁴, m and n are as defined herein.

In some of the embodiments described herein, R¹ is selected from the group consisting of C₁-C₆ alkyl, C₃-C₆ cycloalkyl, C₁-C₆ haloalkyl, phenyl, benzyl, oxtetanyl, oxabicyclo[3.1.0]hexan-6-yl, thienyl and pyrazolyl; wherein R¹ is optionally substituted by: (i) one substituent selected from the group consisting of F, Cl, methyl, hydroxyl, hydroxymethyl, cyano and trifluoromethyl, or (ii) two F substituents. In some embodiments, R¹ is optionally substituted by one or two substituents selected from the group consisting of F, Cl, methyl, ethyl, hydroxyl, hydroxymethyl, methoxymethyl, cyano, trifluoromethyl, difluoromethoxy and trifluoromethoxy. In some embodiments, R¹ is C₁-C₆ alkyl. In some embodiments, R¹ is C₁-C₄ alkyl. In some embodiments, R¹ is C₃-C₅ cycloalkyl. In some embodiments, R¹ is C₃-C₄ cycloalkyl. In some embodiments, R¹ is methyl. In some embodiments, R¹ is ethyl. In some embodiments, R¹ is CF₃CH₂. In some embodiments, R¹ is 2-propyl. In some embodiments, R¹ is tert-butyl. In some embodiments, R¹ is (2-hydroxy)-2-propyl. In some embodiments, R¹ is (2-cyano)-2-propyl. In some embodiments, R¹ is C₁-C₆ haloalkyl. In some embodiments, R¹ is C₁-C₄ haloalkyl. In some embodiment, R¹ preferably is cyclopropyl. In some embodiments, R¹ is mono- or di-fluorocyclopropyl. In some embodiments, R¹ is 1-fluorocyclopropyl. In some embodiments, R¹ is 2-fluorocyclopropyl. In some embodiments, R¹ is 2,2-difluorocyclopropyl. In some embodiments, R¹ is 1-(trifluoromethyl)cyclopropyl. In some embodiments, R¹ is 1-methylcyclopropyl. In some embodiments, R¹ is 1-(hydroxymethyl)cyclopropyl. In some embodiments, R¹ is cyclobutyl. In some embodiments, R¹ is cyclopentyl. In some embodiments, R¹ is phenyl. In some embodiments, R¹ is benzyl. In some embodiments, R¹ is oxetan-3-yl. In some embodiments, R¹ is 3-methyloxetan-3-yl. In some embodiments, R¹ is oxabicyclo[3.1.0]hexan-6-yl. In some embodiments, R¹ is 2-pyridyl. In some embodiments, R¹ is 1-methylpyrazol-4-yl. In some embodiments, R¹ is 2-thienyl.

In some of the embodiments described herein, each R^{N} is independently selected from the group consisting of H and C₁-C₆ alkyl. In some embodiments, each R^{N} is a C₁-C₄ alkyl. In some embodiments, each R^{N} is methyl.

In some of the embodiments described herein, R² preferably is phenyl. In some embodiments, R² is mono- or difluorophenyl. In some embodiments, R² is mono- or dichlorophenyl. In some embodiments, R² is pyridinyl. In some embodiments, R² is chloro substituted pyridinyl. In some embodiments, R² is fluoro substituted pyridinyl. In some embodiments, R² is pyrazolyl. In some embodiments, R² is 1-methyl-1H-pyrazol-4-yl. In some embodiments, R² is 4-chloro-1-methyl-1H-pyrazol-3-yl.

In some of the embodiments described herein, R³ is H. In some embodiments, R³ preferably is F. In some embodiments, R³ is Cl. In some embodiments, R^{3a} and R^{3b} are each methyl. In some embodiments, R³ is methyl. In some embodiments, R³ is OH. In some embodiments, R³ is CN. In some embodiments, R³ is D.

In some of the embodiments described herein, R⁴ is selected from the group consisting of H, F, Cl, CH₃, CH₂CH₃, OCH₃, CF₃, OCF₃, CF₂H, and OCF₂H. In some embodiments, R⁴ preferably is F.

In some of the embodiments described herein, m preferably is 0. In some embodiments, m is 1. In some embodiments, m is 2. In some embodiments, m is 3.

In some of the embodiments described herein, n preferably is 1. In some embodiments, n is 2. In some embodiments, n is 3.

Protecting groups are shown generically in several of the reaction schemes herein, and those skilled in the art will recognize that various different protection and deprotection schemes can in many instances be used alternatively, as described in “Greene's Protective Groups in Organic Synthesis,” Fifth Edition, 2014 by John Wiley& Sons, Inc. In some embodiments, amine or hydroxyl substituents may present in the variables R¹ through R⁴ and R^{N} described herein, and it should be understood that suitable protecting groups may be utilized in association with such substituents.

In some embodiments, a process (P1) for the preparation of a chiral bicyclic ketone compound of formula (I), or a stereoisomer, or a pharmaceutically acceptable salt thereof, wherein R¹, R², R³ and n are as defined herein, comprises:
(a) contacting a compound of chiral N-amino lactam formula p, or a stereoisomer thereof:

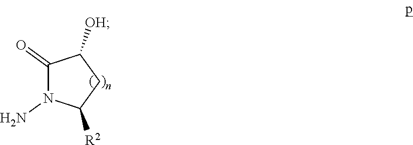

or a salt thereof, in the presence of an acid additive and an alcohol solvent with an imidate compound of formula c:

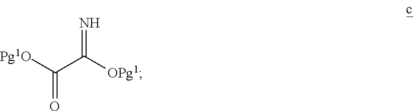

or a salt thereof, to form a chiral bicyclic triazole compound of formula x, or a stereoisomer thereof:

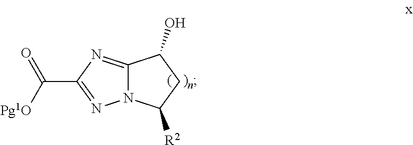

or a salt thereof;
wherein:
   Pg¹ is an optional hydroxyl protecting group and may be the same or different on each occurrence; and
the chiral bicyclic triazole compound of formula x, or the stereoisomer thereof, is an intermediate compound in the preparation of the chiral bicyclic ketone compound of formula (I), or the stereoisomer thereof.

In a preferred embodiment, the chiral N-amino lactam formula p is (3R,5S)-1-amino-3-hyroxy-5-phenylpyrrolidin-2-one. In a preferred embodiment, the imidate compound of formula c is ethyl-2-ethoxy-2iminoacetate. In a preferred embodiment, the chiral bicyclic triazole compound of formula x is ethyl (5S,7R)-7-hydroxy-5-phenyl-6,7-dihydro-5H-pyrrolo[1,2-b][1,2,4]triazole-2-carboxylate.

In some embodiments, the acid additive is a carboxylic acid, a sulfonic acid or an inorganic acid. In some embodiments, the acid additive is acetic acid, oxalic acid, succinic acid, benzoic acid, isobutyric acid, pivalic acid, salicylic acid, oxamic acid, 2-picolinic acid, trifluoroacetic acid, p-toluenesulfonic acid, methanesulfonic acid, formic acid, hydrochloric acid or trimethylsilyl chloride. In a preferred embodiment, the acid additive is acetic acid. In some embodiments, the acid additive is isobutyric acid. In some embodiments, the acid additive is salicylic acid.

In some embodiments, the imidate compound of formula c of step (a) of the process (P1) is replaced by another reagent. In some embodiments, the replacing reagent is ethyl thiooxamate, ethyl cyanoformate, methyl cyanoformate or triethyl 1,3,5-triazine-2,4,6-tricarboxylate. In some embodiments, the replacing reagent is ethyl thiooxamate. In some embodiments, the replacing reagent is triethyl 1,3,5-triazine-2,4,6-tricarboxylate. In some embodiments, the replacing reagent is ethyl thiooxamate and the acid additive is isobutyric acid. In some embodiments, the replacing reagent is triethyl 1,3,5-triazine-2,4,6-tricarboxylate and the acid additive is salicylic acid.

In a preferred embodiment, the yield of the chiral bicyclic triazole compound of formula x of step (a) of the process (P1) is at least 80%. In a particularly preferred embodiment, the yield is at least 85%. In some embodiments, the yield is at least 90%. In some embodiments, the yield is at least 95%. In some embodiments, the yield is at least 98%.

In a preferred embodiment, the alcohol solvent of step (a) of the process (P1) is EtOH. In a particularly preferred embodiment, the acid additive and the alcohol solvent is a mixture of EtOH and acetic acid. In some embodiments, the step (a) of the process (P1) further comprises maintaining a temperature around 60° C. before cooling to the temperature to around 25±10° C. In some embodiments, the step (a) of the process (P1) further comprises adding water. In some embodiments, the step (a) of the process (P1) further comprises adding seeds of the chiral bicyclic triazole compound of formula x.

In some embodiments, the process (P1) further comprises:
(b) deoxyhalogenating the chiral bicyclic triazole compound of formula x, or the stereoisomer thereof, in the presence of a halogenating agent to form a chiral halogenated bicyclic compound of formula y, or a stereoisomer thereof:

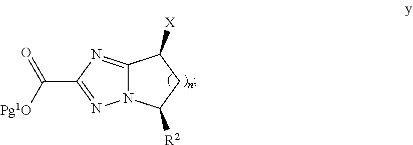

or a salt thereof, wherein X is halogen.

In a preferred embodiment, the chiral halogenated bicyclic compound of formula y is cyclopropyl-[(5S,7S)-7-fluoro-5-phenyl-6,7-dihydro-5H-pyrrolo[1,2-b][1,2,4]triazol-2-yl]methanone. In some embodiments, the halogenating agent is a fluorinating agent. Examples of fluorinating agents are described by M. K. Nielsen et al. in J. Am. Chem. Soc. 140(15):5004-5008 (2018). In some embodiments, the halogenating agent is a sulfonyl fluoride. In a preferred embodiment, the halogenating agent is PBSF. In some embodiments, the halogenating agent is PyFluor (2-pyridinesulfonyl fluoride). In some embodiments, the halogenating agent is diethylaminosulfur trifluoride (DAST). In some embodiments, the halogenating agent is Bis(2-methoxyethyl)aminosulfur trifluoride (Deoxo-Fluor or BAST).

In some embodiments, the step (b) of the process (P1) is performed in the presence of an organic base and an organic solvent. In a preferred embodiment, the organic base is N,N-diisopropylethylamine and the organic solvent is acetonitrile. In some embodiments, an additive is present. In a preferred embodiment, the additive is triethylamine trihydrofluoride. In some embodiments, the additive is N,N-diisopropylethylamine trihydrofluoride. In some embodiments, the additive is acting as a fluoride source.

In some embodiments, the step (b) of the process (P1) further comprises slowly adding reagents over at least one hour at RT to reduce vaporization. In a preferred embodiment, the yield of the chiral halogenated bicyclic compound of formula y of step (a) of the process (P1) is at least 80%. In a particularly preferred embodiment, the yield is at least 85%. In some embodiments, the yield is at least 90%. In some embodiments, the yield is at least 95%. In some embodiments, the yield is at least 98%.

In some embodiments, the process (P1) further comprises:
(c) contacting the chiral halogenated bicyclic compound of formula y, or the stereoisomer thereof, with an acid in the presence of an ethereal solvent/water mixture to form a halogenated bicyclic carboxylic acid compound of formula z, or a stereoisomer thereof:

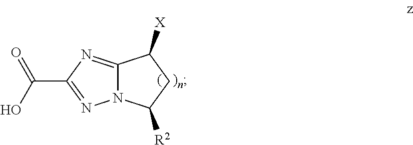

or a salt thereof.

In a preferred embodiment, the halogenated bicyclic carboxylic acid compound of formula z is (5S,7S)-7-fluoro-5-phenyl-6,7-dihydro-5H-pyrrolo[1,2-b][1,2,4]triazole-2-carboxylic acid. In a preferred embodiment, the ethereal solvent/water mixture is a THF/water mixture and the acid is HCl.

In some embodiments, the step (c) of the process (P1) further comprises maintaining a temperature around 50° C. before cooling the temperature to around 35±10° C. In some embodiments, the step (c) of the process (P1) further comprises cooling the temperature to around 20±10° C. and adding water followed by a solution of KOH. In some embodiments, the step (c) of the process (P1) further comprises maintaining the temperature around 30° C. after adding the water and the solution of KOH.

In some embodiments, the process (P1) further comprises:
(d) contacting the halogenated bicyclic carboxylic acid compound of formula z, or the stereoisomer thereof, with a compound of formula aa:

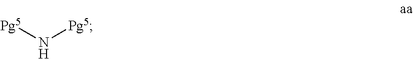

or a salt thereof, in the presence of a coupling agent to form a chiral bicyclic amide compound of formula bb or a stereoisomer thereof:

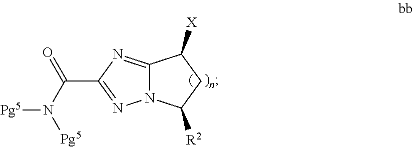

or salt thereof, wherein each Pg⁵ is an amine protecting group and may be the same or different on each occurrence.

In a preferred embodiment, the compound of formula aa is N,O-dimethylhydroxylamine hydrochloride. In a preferred embodiment, the coupling agent is EDCI. In a preferred embodiment, the chiral bicyclic amide compound of formula bb is (5S,7S)-7-fluoro-N-methoxy-N-methyl-5-phenyl-6,7-dihydro-5H-pyrrolo[1,2-b][1,2,4]triazole-2-carboxamide. In some embodiments, the step (d) of the process (P1) further comprises maintaining a temperature around 65° C. In some embodiments, the step (d) of the process (P1) is performed in the presence of an additive. In some embodiments, the additive is NMI.

In some embodiments, the step (d) of the process (P1) further comprises adding seeds of the chiral bicyclic amide compound of formula bb. In a preferred embodiment, the adding seeds of the chiral bicyclic amide compound of formula bb is in the presence of CPME. In some embodiments, the step (d) of the process (P1) further comprises adding an anti-solvent prior to cooling the temperature to around 0° C. In a preferred embodiment, the anti-solvent is heptane.

In some embodiments, the process (P1) further comprises:
(e) contacting the chiral bicyclic amide compound of formula bb or stereoisomer thereof, with a compound of formula cc:

R¹—MgBr_{cc};
or a salt thereof, to form a chiral bicyclic ketone compound dd, or stereoisomer thereof:

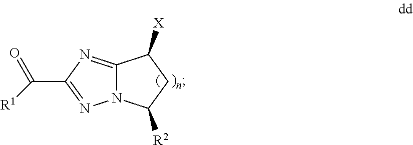

or salt thereof.

In a preferred embodiment, the compound of formula cc is cyclopropylmagnesium bromide. In a preferred embodiment, the chiral bicyclic ketone compound of formula dd is cyclopropyl-[(5S,7S)-7-fluoro-5-phenyl-6,7-dihydro-5H-pyrrolo[1,2-b][1,2,4]triazol-2-yl]methanone. In some embodiments, the alkylation may be carried out in an organic solvent. In some embodiments, the organic solvent is THF in the alkylation step. In some embodiments, the step (e) of the process (P1) further comprises maintaining a temperature around −5° C.±10° C. In some embodiments, the step (e) of the process (P1) further comprises adding seeds of the chiral bicyclic ketone compound of formula dd. In some embodiments, the adding seeds of the chiral bicyclic ketone compound of formula dd is in the presence of an organic solvent. In a preferred embodiment, the organic solvent is EtOH. In a particularly preferred embodiment, the organic solvent is an aqueous solution of EtOH.

In some embodiments, the chiral bicyclic ketone compound of formula (I) is a compound selected from the group consisting of:

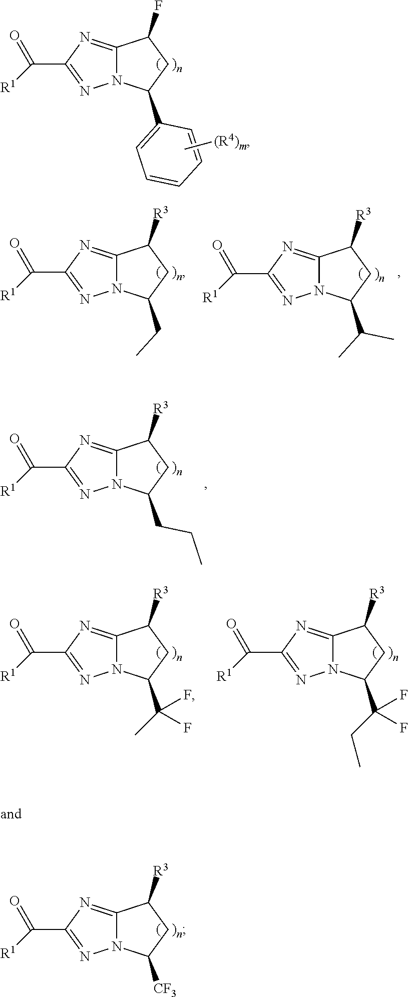

or a pharmaceutically acceptable salt thereof, wherein:
   each R⁴ is selected from the group consisting of H, F, Cl, C₁-C₆ alkyl, C₁-C₆ haloalkyl, C₁-C₆ alkoxy and C₁-C₆ haloalkoxy; and
   m is 0, 1, 2 or 3.

In some embodiments, the chiral bicyclic ketone compound of formula (I) is:

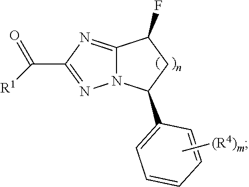

or a pharmaceutically acceptable salt thereof.

In a particularly preferred embodiment, R¹ is cyclopropyl. In a particularly preferred embodiment, m is 0. In a particularly preferred embodiment, n is 1.

In some embodiments, the stereoisomer of the chiral bicyclic ketone compound of formula (I) is a compound of formula (II):

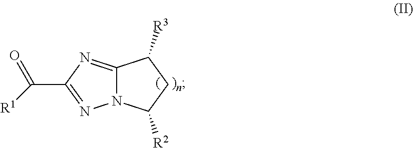

or a pharmaceutically acceptable salt thereof.

In some embodiments, the compound of formula (II) is a compound selected from the group consisting of:

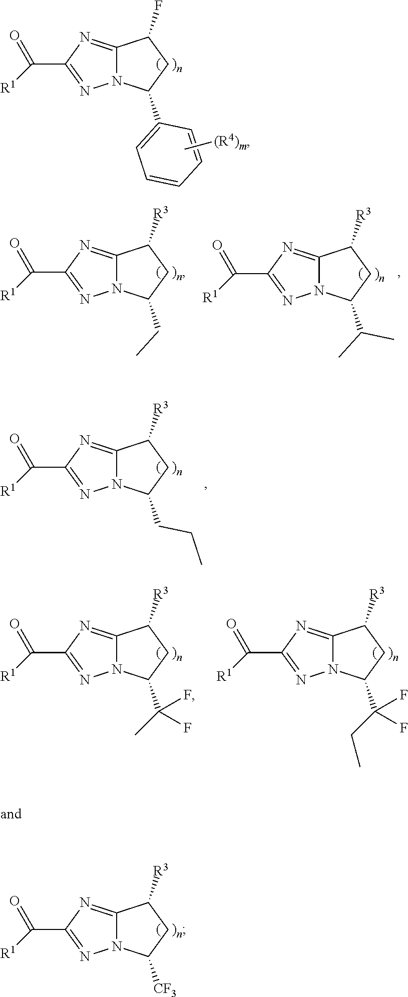

or a pharmaceutically acceptable salt thereof, wherein:
   each R⁴ is selected from the group consisting of H, F, Cl, C₁-C₆ alkyl, C₁-C₆ haloalkyl, C₁-C₆ alkoxy and C₁-C₆ haloalkoxy; and
   m is 0, 1, 2 or 3.

In some embodiments, the compound of formula (II) is:

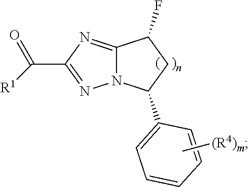

or a pharmaceutically acceptable salt thereof.

In a preferred embodiment, R¹ is cyclopropyl. In a preferred embodiment, m is 0. In a preferred embodiment, n is 1.

In some embodiments, processes are provided herein for the preparation of a chiral bicyclic ketone compound of formula (II), formula (III) or formula (IV):

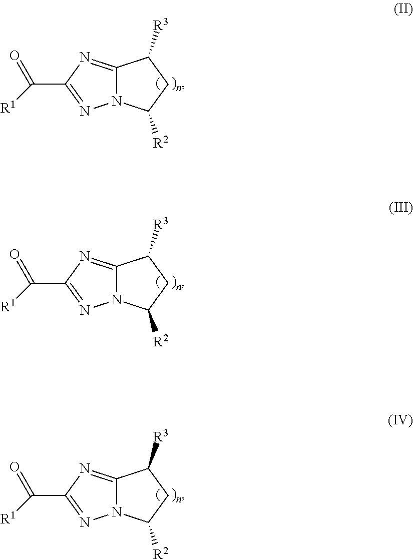

or a pharmaceutically acceptable salt thereof, wherein R¹, R², R³ and n are as defined herein. In a preferred embodiment, R¹ is cyclopropyl. In a preferred embodiment, R² is phenyl. In a preferred embodiment, R³ is F. In a preferred embodiment, n is 1. Each of the compounds of formula (III) and formula (IV) is, for example, a diastereomer of the compound of formula (I) and a diastereomer of the compound of formula (II). The skilled artisan will readily appreciate the various changes and modifications to these processes based on the embodiments described herein.

In some embodiments, a process (P2) for the preparation of a chiral N-amino lactam compound of formula p, or a stereoisomer thereof:

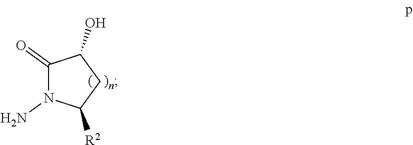

or a salt thereof; wherein R² and n are as defined herein, comprises:
(a) reacting a chiral hydroxydicarboxylic acid compound of formula d or a stereoisomer

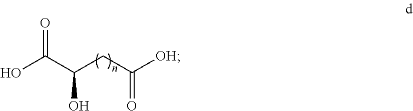

or a salt thereof, in the presence of organic acid chloride of formula e:

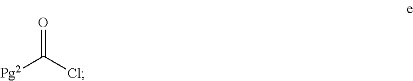

or a salt thereof, to form a chiral carboxylic cyclic anhydride compound of formula f or a stereoisomer thereof:

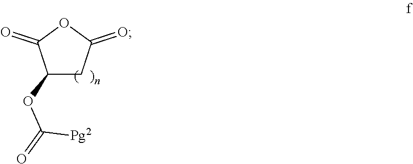

or a salt thereof; and
   (b) reacting a protected hydrazone compound of formula 1, or a stereoisomer thereof:

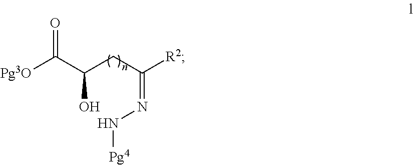

or a salt thereof, in the presence of an acid additive, to form a chiral hydroxy ester hydrazine compound of formula m or a stereoisomer thereof:

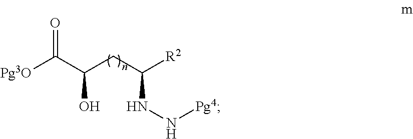

or a salt thereof; wherein:
   Pg² is optionally substituted C₁-C₆ alkyl, C₃-C₆ cycloalkyl or aryl;
   Pg³ is an optional hydroxyl protecting group and may be the same or different on each occurrence;
   Pg⁴ is an optional amine protecting group and may be the same or different on each occurrence;
   the chiral carboxylic cyclic anhydride compound of formula f, or the stereoisomer thereof, is an intermediate in the preparation of the protected hydrazone compound of formula 1, or the stereoisomer thereof; and
   the chiral hydroxy ester hydrazine compound of formula m or the stereoisomer thereof, is an intermediate in the preparation of the chiral N-amino lactam compound of formula p, or the stereoisomer thereof.

In some embodiments, a process for the preparation of the chiral N-amino lactam compound of formula p, or a stereoisomer thereof, or a salt thereof, the process comprising:
(a) reacting a chiral hydroxydicarboxylic acid compound of formula d or a stereoisomer thereof:

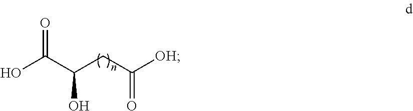

or a salt thereof, in the presence of organic acid chloride of formula e:

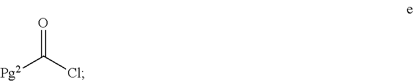

or a salt thereof, to form a chiral carboxylic cyclic anhydride compound of formula f or a stereoisomer thereof:

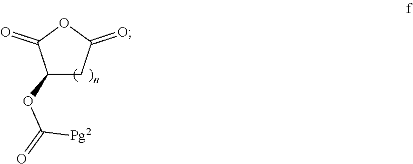

or a salt thereof; wherein:
   Pg², Pg³ and Pg⁴ are as defined herein; and
   the chiral carboxylic cyclic anhydride compound of formula f, or the stereoisomer thereof, is an intermediate in the preparation of the chiral N-amino lactam compound of formula p, or the stereoisomer thereof.

In some embodiments, a process for the preparation of the chiral N-amino lactam compound of formula p, or a stereoisomer thereof, or a salt thereof, the process comprising:
(b) reacting a protected hydrazone compound of formula 1, or a stereoisomer thereof:

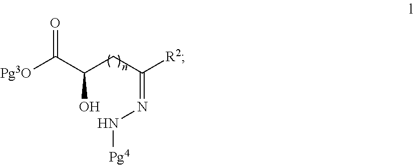

or a salt thereof, in the presence of an acid additive, to form a chiral hydroxy ester hydrazine compound of formula m or a stereoisomer thereof:

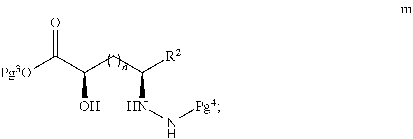

or a salt thereof; wherein:
Pg², Pg³ and Pg⁴ are as defined herein; and
the chiral hydroxy ester hydrazine compound of formula m or the stereoisomer thereof, is an intermediate in the preparation of the chiral N-amino lactam compound of formula p, or the stereoisomer thereof.

In a preferred embodiment, the chiral hydroxydicarboxylic acid compound of formula d is D-malic acid. In a preferred embodiment, the acid chloride solvent of formula e is acetyl chloride. In a preferred embodiment, the chiral carboxylic cyclic anhydride compound of formula f is (S)-(−)-2-acetoxy-succinic anhydride. In some embodiments, the step (a) of the process (P2) further comprises adding i-PrOAc. In some embodiments, the step (a) of the process (P2) further comprises adding n-heptane.

In a preferred embodiment, the protected hydrazone compound of formula 1 is tert-butyl (R,E)-2-(4-ethoxy-3-hydroxy-4-oxo-1-phenylbutylidene)hydrazine-1-carboxylate. In a preferred embody-ment, the chiral hydroxy ester hydrazine compound of formula m is tert-butyl 2-((1S,3R)-4-ethoxy-3-hydroxy-4-oxo-1-phenylbutyl)hydrazine-1-carboxylate. In some embodiments, the acid additive is a carboxylic acid. In some embodiments, the acid additive is oxalic acid, succinic acid, benzoic acid, isobutyric acid, pivalic acid, salicyclic acid, oxamic acid, 2-Picolinic acid, trifluoroacetic acid, formic acid, or acetic acid. In a preferred embodiment, the acid additive is acidic acid. In some embodiments, the acid additive is p-toluenesulfonic acid. In some embodiments, the acid additive is methanesulfonic acid. In some embodiments, the acid additive is hydrochloric acid in ethanol. In some embodiments, the acid additive is trimethylsilyl chloride in ethanol. In some embodiments, the step (b) of the process (P2) is performed in the presence of tetramethylammonium triacetoxyborohydride or sodium triacetoxyborohydride.

In a preferred embodiment, the yield of the chiral carboxylic cyclic anhydride compound of formula f of step (a) of the process (P2) is at least 80%. In some embodiments, the yield is at least 85%. In some embodiments, the yield is at least 90%. In some embodiments, the yield is at least 92%. In a particularly preferred embodiments, the yield is at least 93%.

In a preferred embodiment, the yield of the chiral hydroxy ester hydrazine compound of formula m of step (b) of the process (P2) is at least 70%. In some embodiments, the yield is at least 80%. In a particularly preferred embodiment, the yield is at least 85%. In some embodiments, the yield is at least 90%.

In a preferred embodiment, the diastereomeric ratio (dr) of the chiral hydroxy ester hydrazine compound of formula m of step (b) of the process (P2) to its diastereomer is at least 10:1. In some embodiments, the dr is at least 11:1. In some embodiments, the dr is at least 12:1. In some embodiments, the dr is at least 13:1. In a particularly preferred embodiment, the dr is at least 14:1.

In some embodiments, the process (P2) further comprises:
(c) contacting the chiral carboxylic cyclic anhydride compound of formula f or the stereoisomer thereof, with a reactive arene compound to form a compound of formula h or a stereoisomer thereof:

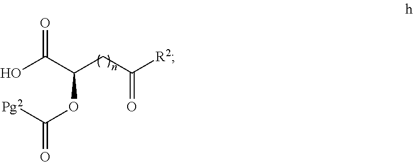

or a salt thereof.

In some embodiments, the reactive arene compound is benzene. In some embodiments, the compound of formula h is (R)-2-acetoxy-4-oxo-4-phenylbutanoic acid. In some embodiments, the contacting step (c) is performed in the presence of a Lewis acid in an organic solvent. In a preferred embodiment, the Lewis acid is AlCl₃ and the organic solvent is CH₂Cl₂. In some embodiments, the organic solvent includes n-heptane.

In some embodiments, the process (P2) further comprises:
(d) reacting the compound of formula h or the stereoisomer thereof, in an alcohol solvent of formula i:

Pg³-OHᵢ;
to form a compound of formula j, or a stereoisomer thereof:

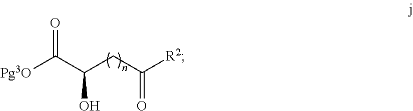

or a salt thereof.

In a preferred embodiment, the alcohol solvent of formula i is EtOH. In some embodiments, the reacting step (d) is performed in the presence of an acid. In some embodiments, the acid is H₂SO₄. In a preferred embodiment, the compound of formula 4 is (−)-ethyl (R)-2-hydroxy-4-oxo-4-phenylbutyrate.

In some embodiments, the process (P2) further comprises:
(e) contacting the compound of formula j, or the stereoisomer thereof, with a hydrazine compound of formula k:

H₂NNH-Pg⁴ₖ.
or a salt thereof, to form the protected hydrazone compound of formula 1, or the stereoisomer thereof, or the salt thereof.

In some embodiments, the contacting step (e) is performed in the presence of an acid additive. In some embodiments, the acid additive is formic acid. In a preferred embodiment, the hydrazine compound of formula k is NH₂NHBoc.

In some embodiments, the process (P2) further comprises:
(f) reacting the protected hydrazone compound of formula 1, or the stereoisomer thereof, to form a chiral protected N-amino lactam compound of formula n, or a stereoisomer thereof:

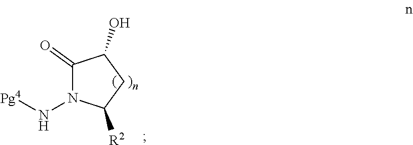

or a salt thereof;
(g) deprotecting the chiral protected N-amino lactam compound of formula n or the stereoisomer thereof, to form a salt compound of formula o or a stereoisomer thereof:

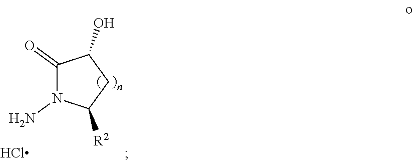

(h) reacting the salt compound of formula o or the stereoisomer thereof, in the presence of a base to form the chiral N-amino lactam compound of formula p, or the stereoisomer thereof.

In a preferred embodiment, the chiral protected N-amino lactam compound of formula n is tert-butyl ((3R,5S)-3-hydroxy-2-oxo-5-phenylpyrrolidin-1-yl)carbamate. In a preferred embodiment, the salt compound of formula o is (3R,5S)-1-amino-3-hydroxy-5-phenylpyrrolidin-2-one hydrochloride. In a preferred embodiment, the chiral N-amino lactam compound of formula p is (3R,5S)-1-amino-3-hydroxy-5-phenylpyrrolidin-2-one. In some embodiments, the step (g) of the process (P2) further comprises adding an acid in an organic solvent. In some embodiments, the acid is HCl and the organic solvent is n-propanol. In some embodiments, the step (g) of the process (P2) further comprises maintaining a temperature at around 20-25° C. In some embodiments, the step (h) of the process (P2) further comprises adding an aqueous solution of a base. In some embodiments, the base is sodium hydroxide.

In a preferred embodiment, the base is NaOH in an aqueous base solution. In some embodiments, the optional amine protecting group Pg⁴ is Boc. In some embodiments, wherein n is 1.

In some embodiments, a process (P3) for the preparation of an imidate salt compound of formula b:

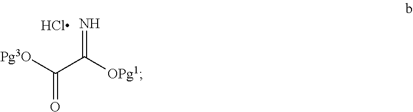

comprises:
   (a) reacting a cyanoformate compound of formula a:

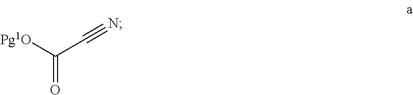

in the presence of an anhydrous acid source in an alcohol solvent to form the imidate salt compound of formula b, wherein the anhydrous acid source is TMSCl, the acid is HCl, and Pg¹ is an optional hydroxyl protecting group and may be the same or different on each occurrence.

In some embodiments, the cyanoformate compound of formula a of step (a) of the process (P3) is replaced by another reagent. In some embodiments, the replacing reagent is ethyl thiooxamate, ethyl cyanoformate, methyl cyanoformate or triethyl 1,3,5-triazine-2,4,6-tricarboxylate. In some embodiments, the replacing reagent is ethyl thiooxamate. In some embodiments, the replacing reagent is triethyl 1,3,5-triazine-2,4,6-tricarboxylate. In some embodiments, the replacing reagent is ethyl thiooxamate. In some embodiments, the replacing reagent is triethyl 1,3,5-triazine-2,4,6-tricarboxylate.

In a preferred embodiment, the alcohol solvent is EtOH in MTBE.

In a preferred embodiment, the yield of the imidate salt compound of formula b of step (a) of the process (P3) is at least 65%. In some embodiments, the yield is at least 70%. In some embodiments, the yield is at least 75%. In a particularly preferred embodiments, the yield is at least 78%.

In some embodiments, a process (P4) for the preparation of a hydroxyketoester compound of formula j, or a stereoisomer thereof:

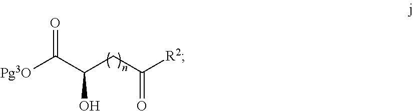

comprises:
(a) reacting a diketoester compound of formula hh:

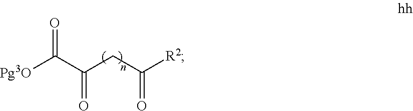

in the presence of a ketoreductase (KRED) to form the hydroxyketoester compound of formula j, or a stereoisomer thereof, wherein R², Pg³ and n are as defined herein.

In some embodiments, the process (P4) further comprises:
(b) reacting the hydroxyketoester compound of formula j, or a stereoisomer thereof, to form a protected hydrazone compound of formula 1, or a stereoisomer thereof:

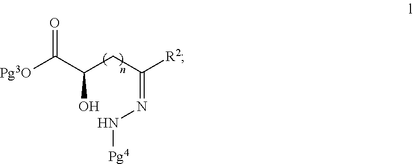

or a salt thereof.

In a preferred embodiment, the KRED is an engineered ketoreductase. In a more preferred embodiment, the engineered ketoreductase is ADH-114 (c-LEcta GmbH, Germany) or 1-200-0-16 (Porton Pharma Solutions Ltd, China).

In a preferred embodiment, the hydroxyketoester compound of formula j is (−)-ethyl (R)-2-hydroxy-4-oxo-4-phenylbutyrate. In a preferred embodiment, the diketoester compound of formula hh is ethyl 2,4-dioxo-4-phenyl-butanoate. In a preferred embodiment, the protected hydrazone compound of formula 1 is tert-butyl (R,E)-2-(4-ethoxy-3-hydroxy-4-oxo-1-phenylbutylidene)hydrazine-1-carboxylate

In a preferred embodiment, the KRED is highly stereoselective. In some embodiments, stereomeric excess of the hydroxyketoester compound of formula j of step (a) of the process (P4) is at least 80%. In some embodiments, the stereomeric excess is at least 85%. In a preferred embodiment, the stereomeric excess is at least 90%. In a more preferred embodiment, the stereomeric excess is at least 95%. In a particularly preferred embodiment, the stereomeric excess is 99%.

In a preferred embodiment, the yield of the hydroxyketoester compound of formula j of step (a) of the process (P4) is at least 80%. In some embodiments, the yield is at least 85%. In some embodiments, the yield is at least 90%. In some embodiments, the yield is at least 92%. In a preferred embodiment, the yield is at least 93%. In a particularly preferred embodiment, the yield is at least 95%.

In a preferred embodiment, the yield of the protected hydrazone compound of formula 1 of step (b) of the process (P4) is at least 80%. In some embodiments, the yield is at least 85%. In some embodiments, the yield is at least 90%. In some embodiments, the yield is at least 92%. In a preferred embodiment, the yield is at least 93%. In a particularly preferred embodiment, the yield is at least 95%.

In some embodiments, the process (P4) further comprises the presence of an alcohol. For example, the alcohol is used in regeneration of the cofactor. In a preferred embodiment, the process (P4) comprises the presence of the alcohol without a coenzyme being present. In some embodiments, the alcohol is a secondary alcohol. For example, secondary alcohols include lower secondary alkanols and aryl-alkyl carbinols. Examples of lower secondary alcohols include isopropanol, 2-butanol, 3-methyl-2-butanol, 2-pentanol, 3-pentanol, and the like. In a preferred embodiment, the secondary alcohol is isopropanol. Examples of alkyl-alkyl carbinols include unsubstituted and substituted 1-arylethanols. In some embodiments, the secondary alcohol is the R-enantiomer of a chiral secondary alcohol. In other embodiments, the secondary alcohol is the —S-anantiomer of a chiral secondary alcohol. In some embodiments, the process (P4) further comprises the presence of a coenzyme. In a preferred embodiment, the coenzyme is a glucose dehydrogenase. In a more preferred embodiment, the glucose dehydrogenase is GDH-105 (Codexis, Inc., California, USA) or 1-030-0-05 (Porton Pharma Solutions Ltd, China). In some embodiments, the KRED is provided in an immobilized form or in form of a whole cell.

In some embodiments, any step of the processes (P1-P4) is scalable. In a preferred embodiment, at least one step of the processes (P1-P4) is scalable to at least a kilogram scale.

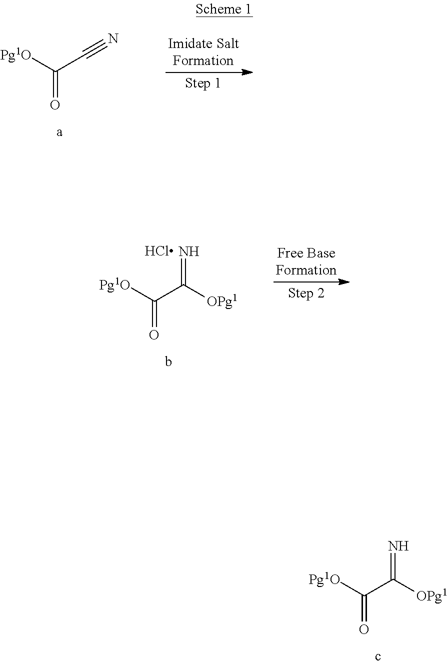

Referring now to Scheme 1, synthesis of an intermediate imidate compound c is shown, wherein Pg¹ is as defined herein. In step 1 of Scheme 1, a cyanoformate compound a undergoes imidate salt formation to afford an imidate salt compound b. Salt formation of step 1 may be carried out with an anhydrous acid source, e.g., TMSCl for HCl, in the presence of an alcohol, e.g., EtOH, in an ethereal solvent, e.g., MTBE. In some embodiments, the dry source used in this step is TMSCl and the acid is HCl.

In step 2, the imidate salt is transformed to the free base compound c with an organic base. In some embodiments, the organic base is triethylamine in this step. In some embodiments, the transformation may be carried out in the presence of a drying agent in an organic solvent. In some embodiments, the drying agent is Na₂SO₄ in this step. In some embodiments, the organic solvent is MTBE in this step. Compound e may then be used as shown below in Scheme 3A or 3B.

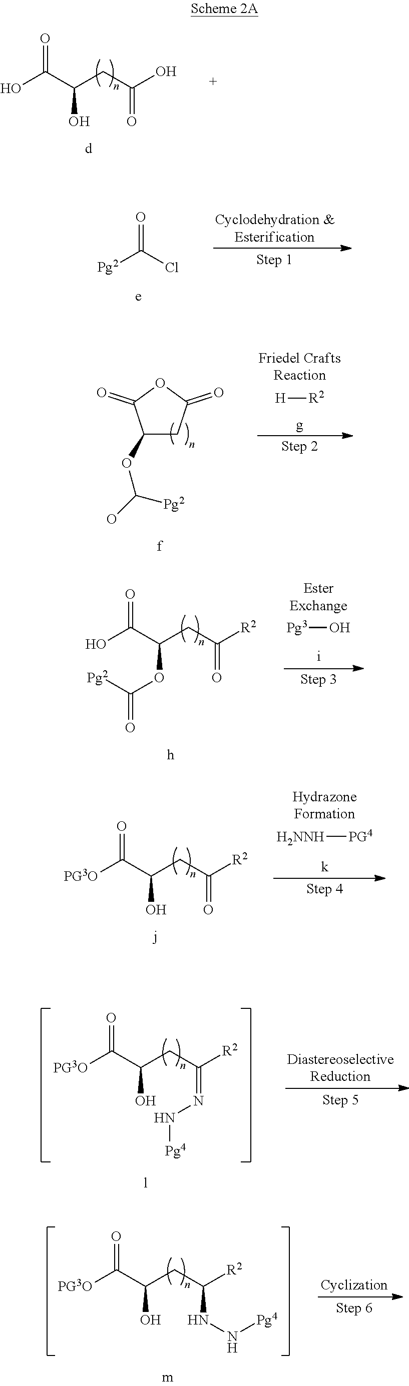

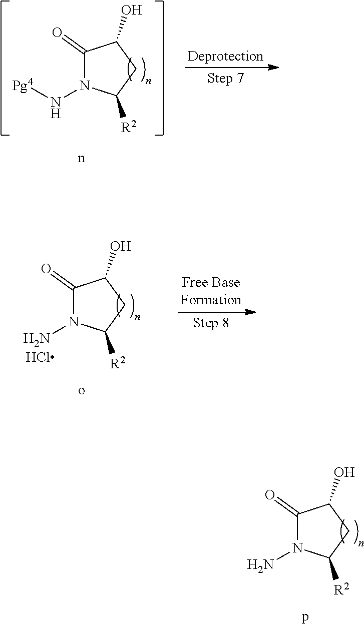

Scheme 2A illustrates the synthesis of a chiral N-amino lactam compound p, wherein Pg², Pg³, Pg⁴, R² and n are as defined herein.

In step 1 of Scheme 2A, a chiral hydroxydicarboxylic acid compound d undergoes cyclodehydration and esterification to afford a chiral carboxylic dihydrofurandione compound f in organic acid chloride e. In some embodiments, the acid chloride solvent e is acetyl chloride in this step.

In step 2 of Scheme 2A, the compound f undergoes a Friedel Crafts reaction in the presence of a reactive arene compound g to afford compound h. In some embodiments, the Friedel Crafts reaction is performed in the presence of a Lewis acid in an organic solvent. In some embodiments, the arene compound g is benzene. In some embodiments, the Lewis acid is AlCl₃ in this step. In some embodiments, the organic solvent is CH₂Cl₂ in this step.

In step 3 of Scheme 2A, the compound h undergoes an ester exchange to afford compound j in an alcohol solvent i. In some embodiments, the ester exchange may be carried out in the presence of an acid. In some embodiments, the alcohol solvent i is EtOH in this step. In some embodiments, the acid is H₂SO₄ in this step.

In step 4 of Scheme 2A, compound i undergoes a hydrazone formation to afford a protected hydrazone compound 1. In some embodiments, the hydrazone formation may be carried out in the presence of an acid additive in this step. In some embodiments, the acid additive is formic acid in this step. In some embodiments, the protecting group Pg⁴ is Boc in this step.

In step 5 of Scheme 2A, the hydrazone compound 1 undergoes a diastereoselective reduction using a reducing agent to afford a chiral hydrazine compound m. In some embodiments, the reduction may be carried out in the presence of an acid additive in an organic solvent. In some embodiments, the reducing agent is Me₄NBH(OAc)₃ or NaBH(OAc)₃ in this step. In some embodiments, the acid additive is AcOH in this step. In some embodiments, the organic solvent is CH₂Cl₂ in this step.

In step 6 of Scheme 2A, the chiral hydrazine compound m undergoes a cyclization to afford a chiral protected N-amino lactam compound n. In some embodiments, the cyclization may be carried out in an alcohol solvent upon heating. In some embodiments, the alcohol solvent is EtOH in this step.

In step 7 of Scheme 2A, the chiral protected N-amino lactam compound n undergoes deprotection to afford a salt o of the target compound p. In some embodiment, the salt o is a HCl salt in this step.

In step 8 of Scheme 2A, the salt o is freebased to afford the N-amino lactam compound p in the presence of a base. In some embodiments, the base is NaOH in this step. In some embodiments, the reaction of step 8 may be carried out in an aqueous base solution.

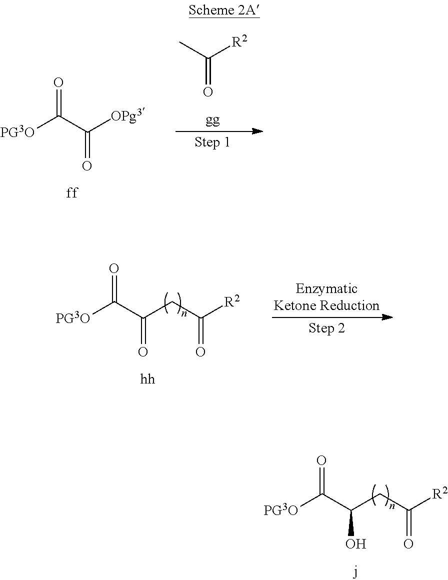

Scheme 2A′ illustrates the synthesis of hydroxyketoester compound j, wherein Pg³, R² and n are as defined herein, and Pg³ is an optional hydroxyl protecting group and may be the same or different on each occurrence. In some embodiments, Pg³ is Pg³.

In step 1 of Scheme 2A′, an oxalate diester compound ff undergoes condensation to afford a diketoester compound hh in the presence of aryl methyl ketone compound gg. In some embodiments, the aryl methyl ketone compound gg is acetophenone in this step.

In step 2 of Scheme 2A′, the diketoester compound hh undergoes an enzymatic ketone reduction to afford hydroxyketoester compound j. In some embodiments, the enzymatic reduction is performed in the presence of a ketoreductase (KRED). In some embodiments, the KRED is highly stereoselective. In some embodiments, KRED is an engineered ketoreductase. In a more preferred embodiment, the engineered ketoreductase is ADH-114 (c-LEcta GmbH, Germany) or 1-200-0-16 (Porton Pharma Solutions Ltd, China).

In some embodiments of step 2 of Scheme 2A′, the enzymatic reduction is further performed in the presence of a cofactor. In some embodiments, the cofactor is NAD, NADH, NADP or NADPH. In some embodiments of step 2 of Scheme 2A′, the enzymatic reduction is further performed in the presence of a coenzyme. In some embodiments, the coenzyme is glucose dehydrogenase. In some embodiments, the glucose dehydrogenase is GDH-105 (Codexis, Inc., California, USA) or 1-030-0-05 (Porton Pharma Solutions Ltd, China). In some embodiments of step 2 of Scheme 2A′, the enzymatic reduction is further performed in the presence of an alcohol. In some embodiments, the alcohol is ethanol. In some embodiments of step 2 of Scheme 2A′, the enzymatic reduction is performed in the presence of the alcohol without a coenzyme being present.

In some embodiments, step 2 of Scheme 2A′ further comprises the presence of D-(+)-glucose. In some embodiments, step 2 of Scheme 2A′ further comprises the presence of an additive and/or an organic cosolvent. In some embodiments, the organic cosolvent is ethanol or acetonitrile in this step. In some embodiments, the additive is NaOAc, MgCl₂·6H₂O, Na₂CO₃, or NaOH in this step. In some embodiments, step 2 of Scheme 2A′ is performed in a buffer solution at a controlled pH and temperature. In some embodiments, the buffer solution includes K₂HPO₄, KH₂PO₄, and water. In some embodiments, the controlled pH is within the range of 6-9. In a preferred embodiment, the controlled pH is 6.5 or 7. In some embodiments, the temperature is within the range of 20-40° C. In a preferred embodiment, the temperature is 25° C. or 30° C.

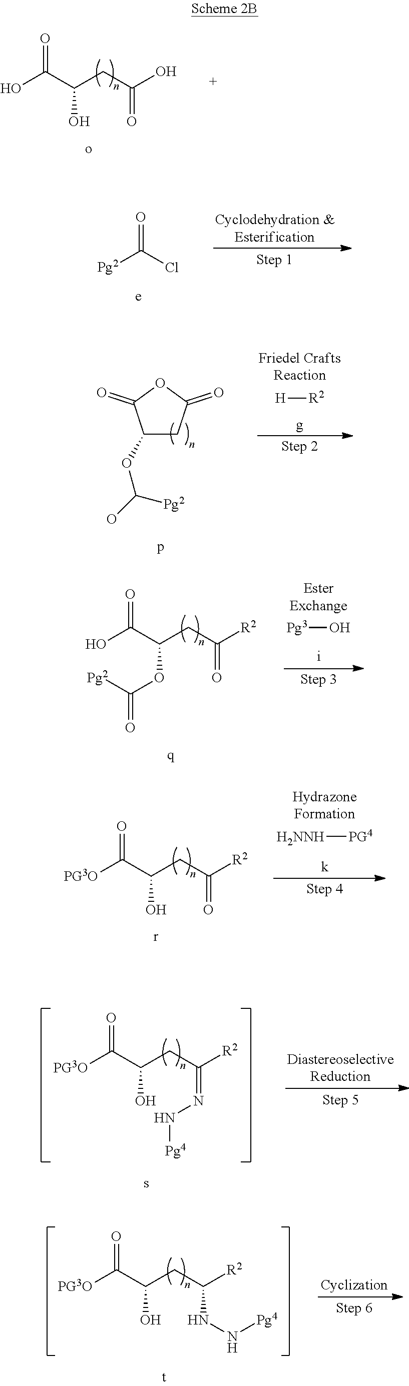

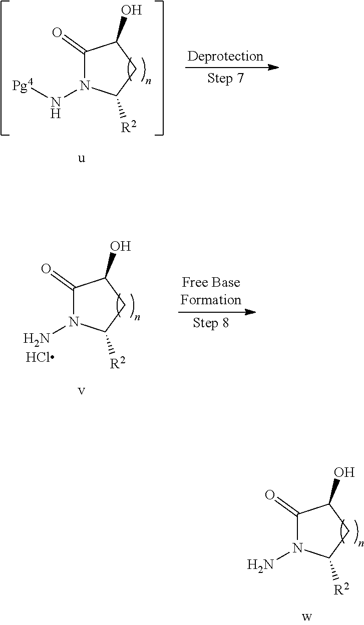

Scheme 2B illustrates the synthesis of a chiral N-amino lactam compound w, wherein Pg², Pg³, Pg⁴, R² and n are as defined herein. Steps 1-7 of Scheme 2B are similar to the steps 1-7 of Scheme 2A with step 1 starting from a chiral hydroxydicarboxylic acid compound o which result in chiral compounds p, q, r, s, t, u, v and the chiral N-amino lactam compound w.

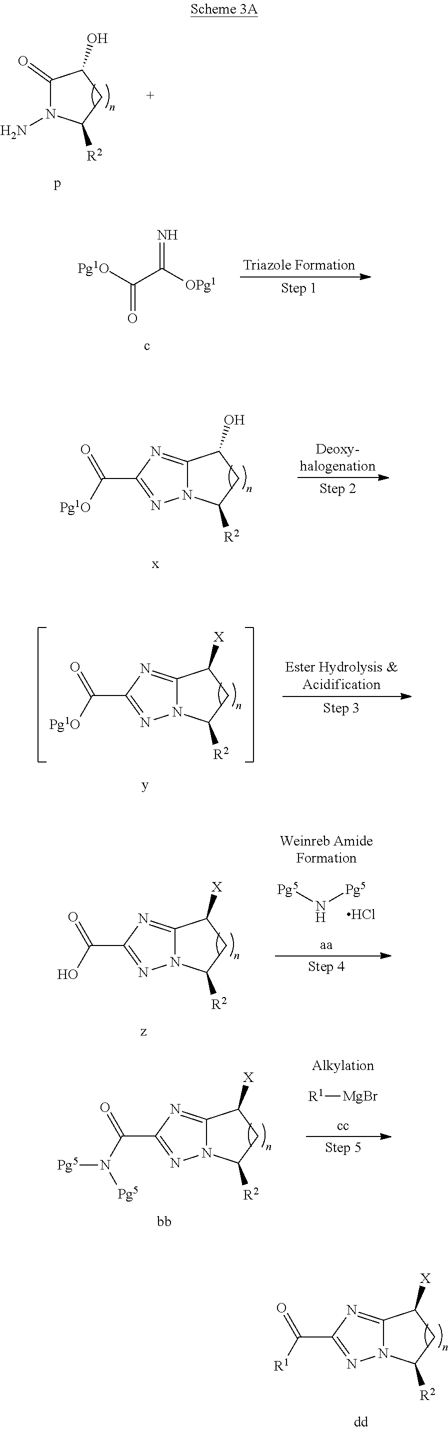

Scheme 3A illustrates the synthesis of a chiral bicyclic ketone compound dd, wherein Pg¹, Pg⁵, R¹, R², X and n are as defined herein. In some embodiments, the chiral bicyclic ketone compound dd is a chiral 6,7-dihydro-5H-pyrrolo[1,2-b][1,2,5]triazole ketone.

In step 1 of Scheme 3A, the compounds p and c are combined to undergo a triazole formation to afford a chiral bicyclic triazole compound x. In some embodiments, the triazole formation may be carried out in the presence of an acid additive and an alcohol solvent. In some embodiments, the acid additive is acetic acid in this step. In some embodiments, the alcohol solvent is ethanol in this step.

In step 2 of Scheme 3A, the chiral bicyclic triazole compound x undergoes deoxyhalogenation in the presence of a halogenating agent to afford a chiral halogenated bicyclic compound y. In some embodiments, the deoxyhalogenation may be carried out in the presence of an organic base in an organic solvent. In some embodiments, the deoxyhalogenation includes deoxyfluorination in the presence of a fluorinating agent. In a preferred embodiment, the fluorinating agent is PBSF in this step. In some embodiments, the fluorinating agent is PyFluor (2-pyridinesulfonyl fluoride) in this step. In a preferred embodiment, the organic base is N,N-diisopropylethylamine in this step. In some embodiments, the organic solvent is acetonitrile. In a preferred embodiment, an additive is present. In a particularly preferred embodiment, the additive is triethylamine trihydrofluoride. In some embodiments, the additive is N,N-diisopropylethylamine trihydrofluoride. In some embodiments, the additive is acting as a fluoride source. In some embodiments, the deoxyhalogenation is carried out by slowly adding reagents over at least one hour at RT to reduce vaporization.

In step 3 of Scheme 3A, the chiral halogenated bicyclic compound y undergoes ester hydrolysis and acidification to afford a halogenated bicyclic carboxylic acid compound z. In some embodiments, the ester hydrolysis and acidification may be carried out in the presence of an ethereal solvent/water mixture with an acid. In some embodiments, the solvent/water mixture is a THF/water mixture in this step. In some embodiments, the acid is HCl in this step.

In step 4 of Scheme 3A, the halogenated bicyclic carboxylic acid compound z undergoes Weinreb amide formation with an amide aa to afford a chiral bicyclic amide bb. In some embodiments, the Weinreb amide formation may be carried out in the presence of a coupling agent. In some embodiments, the amide formation may be carried out in the presence of an additive in an organic solvent. In some embodiments, the amide aa is N,O-dimethylhydroxylamine in this step. In some embodiments, the coupling reagent is EDCI in this step. In some embodiments, the additive is N-methylimidazole and the organic solvent is CH₂Cl₂ in this step.

In step 5 of Scheme 3A, the chiral bicyclic amide bb undergoes alkylation in the presence of an organometallic reagent cc to afford the chiral target compound dd. In some embodiments, the alkylation may be carried out in an organic solvent. In some embodiments, the organometallic reagent cc is alkylmagnesium bromide in this step. In some embodiments, the alkylmagnesium bromide is cyclopropylmagnesium bromide in this step. In some embodiments, the organic solvent is THF in this step. In some embodiments, this step further comprises adding seeds of the chiral bicyclic ketone compound of formula dd. In some embodiments, the adding seeds of the chiral bicyclic ketone compound of formula dd is in the presence of an organic solvent. In a preferred embodiment, the organic solvent is EtOH. In a particularly preferred embodiment, the organic solvent is an aqueous solution of EtOH.

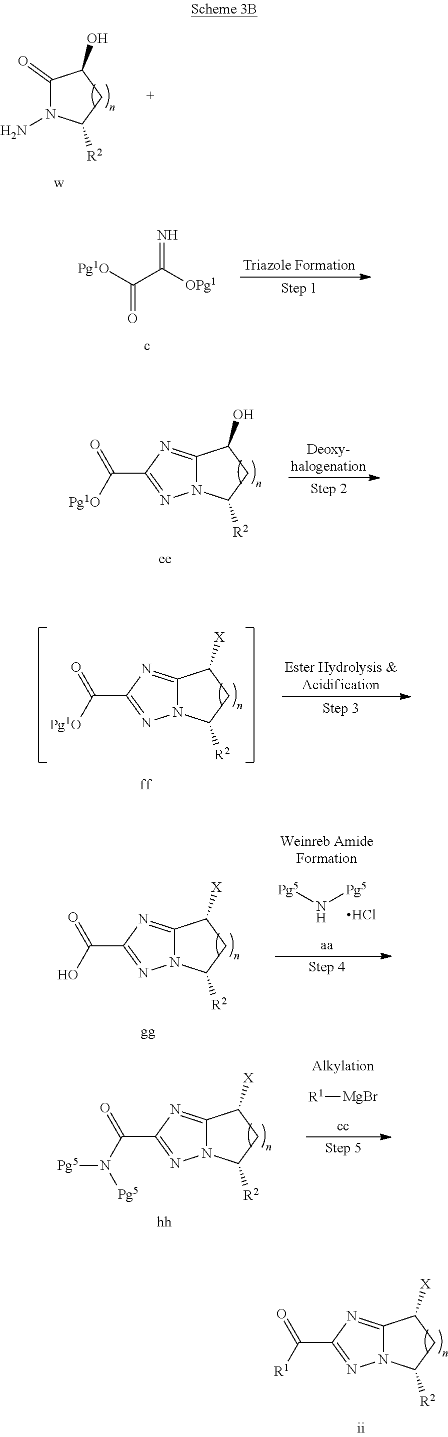

Scheme 3B illustrates the synthesis of a chiral bicyclic ketone compound ii, wherein Pg¹, Pg⁵, R¹, R², X and n are as defined herein. Steps 1-5 of Scheme 3B are similar to the steps 1-5 of Scheme 3A with step 1 starting from compounds w and c, which result in chiral compounds ee, ff, gg, gg and the chiral bicyclic ketone compound ii.

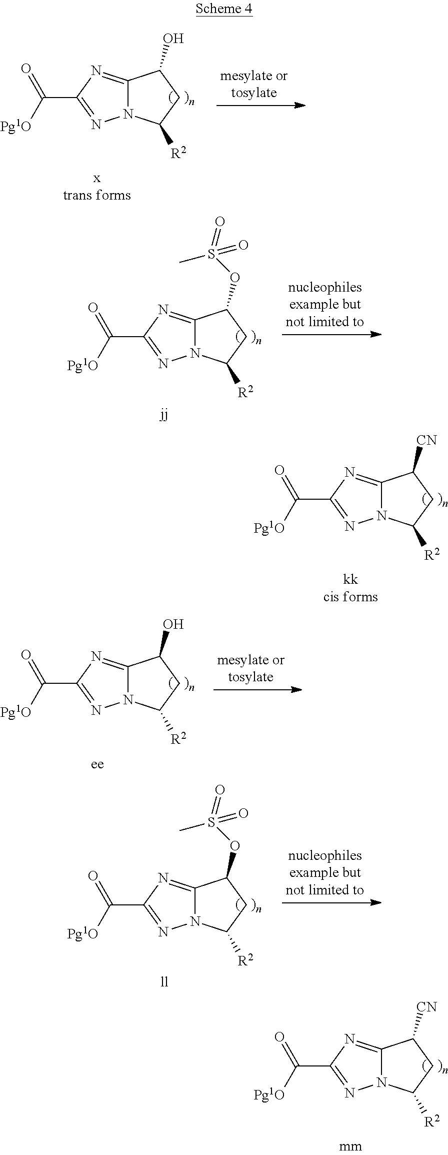

Scheme 4 illustrates the synthesis to prepare additional bicyclic ring diversity of compounds of formulas (I)-(IV) using a variety of nucleophiles including but not limited to halide and cyanide sources.

Many variations on the reactions of Schemes 1 through 4 are possible and will suggest themselves to those skilled in the art. For example, the order of the reactions may be varied in many embodiments. In some examples, the reactions include stereoisomers of the compounds shown in the Schemes described herein. In some instances, reaction products need not be isolated but can be used in situ in the following reaction. The amine and hydroxyl protecting group chemistry, as well as the timing of protection and deprotection events, may, for example, be varied from the particular embodiments described herein.

### EXAMPLES

The invention will be more fully understood by reference to the following examples. They should not, however, be construed as limiting the scope of the invention.

These examples serve to provide guidance to a skilled artisan to prepare and use the compounds, compositions and methods of the invention. While particular embodiment of the present invention are described, the skilled artisan will appreciate that various changes and modifications can be made without departing from the spirit and scope of the inventions.

The chemical reactions in the examples described can be readily adapted to prepare a number of other compounds of the invention, and alternative methods for preparing the compounds of this invention are deemed to be within the scope of this invention. For example, the synthesis of non-exemplified compounds according to the invention can be successfully performed by modifications apparent to those skilled in the art, for example, by appropriately protecting interfering group, by utilizing other suitable reagents known in the art, for example, by appropriately protecting interfering groups by utilizing other suitable reagents known in the art other than those described, and/or by making routine modifications of reaction conditions.

In the examples below, unless otherwise indicated all temperatures are set forth in degrees Celsius. Commercially available reagents were purchased from suppliers such as Aldrich Chemical Company, Lancaster, TCJ or Maybridge and were used without further purification unless otherwise indicated. The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried. ¹H NMR spectra were obtained in deuterated CDCl₃, d₆-DMSO, CH₃OD or d₆-acetone solvent solutions (reported in ppm) using or trimethylsilane (TMS) or residual non-deuterated solvent peaks as the reference standard. When peak multiplicities are reported, the following abbreviates are used: s (singlet), d (doublet), t (triplet), q (quartet), m (multiplet, br (broadened), dd (doublet of doublets), dt (doublet of triplets). Coupling constants, when given, are reported in Hz (Hertz).

All abbreviations used to describe reagents, reaction conditions or equipment are intended to be consistent with the definitions set forth in the following list of Abbreviations. The chemical names of discrete compounds of the invention were typically obtained using the structure naming feature of ChemDraw naming program.

### Abbreviations

ACN Acetonitrile
AcOH Acetic Acid
Boc tert-Butoxycarbonyl
CPME Cyclopentyl Methyl Ether
DAST Diethylaminosulfur trifluoride
DCE 1,2-Dichloroethane
DCM Dichloromethane
DIPEA or i-Pr₂NEt N,N-Diisopropylethylamine
DMF N,N-Dimethylformamide
DMSO Dimethyl sulfoxide
DPPH 2,2-Diphenyl-1-picrylhydrazyl
EDCI 1-Ethyl-3-(3-dimethylaminopropyl)-carbodiimide
ET External Temperature
Et₃N·3HF Triethylamine trihydrofluoride
EtOH Ethanol
GC Gas Chromatography
HPLC High Pressure Liquid Chromatography
IT Internal Temperature
KF Karl-Fischer Titration
KRED Ketoreductase
LCMS Liquid Chromatography Mass Spectrometry
2-MeTHF 2-Methyltetrahydrofuran
MTBE Methyl tert-butyl ether
NAD Nicotinamide Adenine Dinucleotide
NADH Reduced Nicotinamide Adenine Dinucleotide
NADP Nicotinamide Adenine Dinucleotide Phosphate
NADPH Reduced Nicotinamide Adenine Dinucleotide Phosphate
NMI N-Methylimidazole
NMR Nuclear Magnetic Resonance
PBS Phosphate Buffered Solution
PBSF Perfluorobutanesulfonyl fluoride
PCC Pyridinium chlorochromate
RP Reverse phase
RT or R_{T} Retention time
SEM 2-(Trimethylsilyl)-ethoxymethyl
SFC Supercritical Fluid Chromatography
TBAH Tetrabutylammonium triacetoxyborohydride
TFA Trifluoroacetic acid
THF Tetrahydrofuran
TMSCl Trimethylsilyl chloride

### Example 1: Preparation of ethyl-2-ethoxy-iminoacetate hydrochloride A3

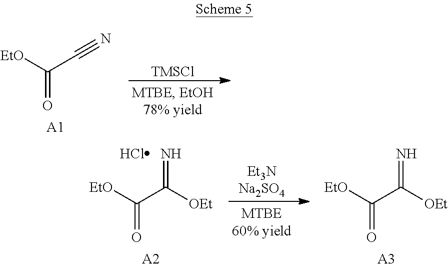

The synthesis of ethyl-2-ethoxy-iminoacetate A3 is illustrated in Scheme 5.

### Ethyl-2-ethoxy-iminoacetate hydrochloride (A2)

To a reactor was charged MTBE (28.0 kg, 6 V, KF: 360 ppm), ethyl cyanoformate A1 (6.3 kg, 63.6 mol, 1.0 equiv), and TMSCl (21.4 kg, 197.1 mol, 3.1 equiv) at RT (˜30° C.) under N₂ atmosphere. The mixture was cooled to 0-5° C. EtOH (12.0 kg, 4.1 equiv, KF: 200 ppm) was added dropwise at 0-5° C. over 30 min. Upon the completion of the addition, the mixture was warmed to 5-10° C. and then stirred for 23 h. The reaction was monitored by GC (ethyl cyanoformate A1 <5 A %). The mixture was filtered under N₂ atmosphere and the cake was washed with MTBE (17 kg×3, 3.7 V×3). The cake was dried under N₂ flow at 25-30° C. for 4 h to give product A2 (9.0 kg, 78% yield) as a white powder.

### Ethyl-2-ethoxy-iminoacetate hydrochloride (A3)

To a reactor was charged Na₂SO₄ (9.0 kg, 100 wt %), A2 (9.0 kg, 49.5 mol, 1.0 equiv), and MTBE (953.0 kg, 9.0 V) at RT (˜30° C.) under N₂ atmosphere. The mixture was cooled to 0° C. A solution of Et₃N (5.4 kg, 1.08 equiv, KF: 360 ppm) in MTBE (6.7 kg, 1 V, KF: 300 ppm) was added dropwise at 0-5° C. over 2 h. Upon the completion of the addition, the mixture was warmed to 20° C. and then stirred for 4 h. The reaction was monitored by GC (cpd. 2<0.5 A %). The mixture was filtered under N₂ atmosphere and the cake was washed with MTBE (7.5 kg, 1.1 V). The filtrate was concentrated under vacuum and dried in vacuo at 20° C. to give product A3 as a liquid, which was combined with other batches (˜60% yield).

¹H NMR (400 MHz, CDCl₃) δ 8.77 (s, 1H), 4.30 (dq, J=9.6, 7.1 Hz, 4H), 1.36 (dt, J=8.2, 7.1 Hz, 6H). ¹³C NMR (100 MHz, CDCl₃) δ 159.4, 158.0, 63.4, 63.0, 14.0, 13.9.

### Example 2: Preparation of (3R,5S)-1-amino-3-hyroxy-5-phenylpyrrolidin-2-one A12

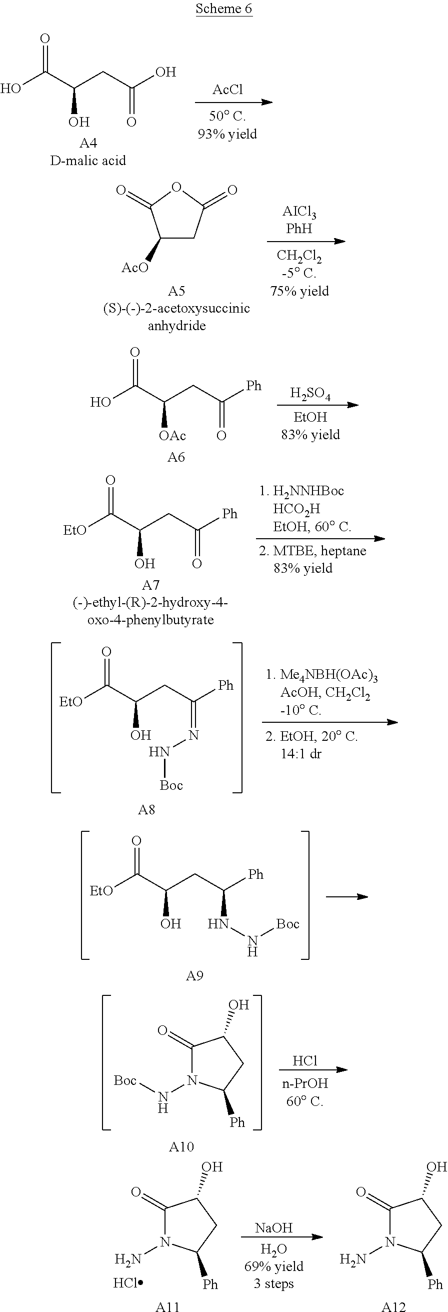

The synthesis of (3R,5S)-1-amino-3-hyroxy-5-phenylpyrrolidin-2-one A12 is illustrated in Scheme 6.

### (S)-(−)-2-acetoxysuccinic anhydride (A5)

D-malic acid A4 (12.0 kg, 89.5 mol, 1.0 equiv) in acetyl chloride (52.8 kg, 4 V) was heated to 40-53° C. (IT) and stirred for 16 h. The reaction was monitored by HPLC (D-malic acid A4<0.5 A %). The reaction mixture was concentrated to remove volatiles at 40-60° C. (ET) under vacuum. The crude oil was dissolved in i-PrOAc (10.6 kg, 1.0 V). An inert solvent n-heptane (27.7 kg, 3.3 vol.) was added at 15° C. After the addition of n-heptane was completed, solids were precipitated out. The suspension was stirred at 0° C. for another 1 h before the solids were filtered and rinsed with n-heptane (8.3 kg×2, 2 V×2). The cake was dried at 40-45° C. under vacuum to yield (S)-(−)-2-acetoxysuccinic anhydride A5 that is consistent with commercially available samples and literature reports (see, e.g., Shiuey, S. J.; Partridge, J. J.; Uskokovic, M. R. J. Org. Chem. 1988, 53, 1040-1046).

¹H NMR (400 MHz, CDCl₃) δ 5.53 (dd, J=9.6, 6.3 Hz, 1H), 3.38 (dd, J=18.9, 9.6 Hz, 1H), 3.02 (dd, J=18.9, 6.3 Hz, 1H), 2.19 (s, 3H).

### (R)-2-acetoxy-4-oxo-4-phenylbutanoic acid (A6)

A suspension of anhydrous aluminium chloride (25.3 kg, 189.8 mol, 2.5 equiv) in CH₂Cl₂ (320.0 kg, 20 V) was stirred under N₂ at −10° C. to −5° C. (S)-(−)-2-acetoxysuccinic anhydride A5 (12.0 kg, 75.9 mol, 1.0 equiv) was added over 15 min. The mixture was stirred at −10° C. to −5° C. for 10 min under N₂. Benzene (18.7 kg, 239.1 mol, 3.15 equiv) was added to the mixture dropwise over 30-90 min at −10° C. to −5° C. under N₂. The mixture was stirred for 18 h at −5° C. to 0° C. under N₂. The reaction was monitored by HPLC (anhydride <0.5 A %). The mixture was quenched with aq. HCl (90.0 kg, 3.0 M, 3.5-3.6 equiv). The organic phase was settled by standing for 2 h and was then separated from the suspension. The aqueous phase was extracted with i-PrOAc (52.8 kg×2, 5 V×2). All organic phases were combined and washed with brine (68 kg×2, 5 V×2). The organic phase was concentrated under vacuum at 35-40° C. The crude was dissolved in i-PrOAc (105.6 kg, 10 V) at 40° C. for 30 min. The suspension was filtered through celatom (2.4 kg, 20 wt %) and the cake was washed with i-PrOAc (10.6 kg, 1 V). The filtrate was concentrated under vacuum at 35-40° C. The crude was slurried in a mixture of CH₂Cl₂/n-heptane (15.6 kg/42.0 kg, 2 V/10 V), filtered, and washed with n-heptane (10.6 kg, 1 V), and then dried at 30-35° C. to yield (R)-2-acetoxy-4-oxo-4-phenylbutanoic acid A6 (96.1 kg, 70% yield) as a white powder, which is consistent with commercially available samples and literature reports (see, e.g., Wilkins, T. D.; Tucker, K. D. Process for Producing Optically Active 2-Hydroxy-4-Arylbutyric Acid or its Ester. U.S. Pat. No. 5,959,139, Sep. 28, 1999).

¹H NMR (400 MHz, CDCl₃) δ 11.26 (br s, 1H), 8.01-7.92 (m, 2H), 7.61 (t, J=7.4 Hz, 1H), 7.49 (t, J=7.7 Hz, 2H), 5.74 (dd, J=7.8, 3.5 Hz, 1H), 3.66 (dd, J=17.8, 7.8 Hz, 1H), 3.54 (dd, J=17.8, 3.6 Hz, 1H), 2.11 (s, 3H). ¹³CNMR (101 MHz, CDCl₃) δ 194.7, 174.8, 170.1, 136.0, 133.8, 128.8, 128.2, 67.3, 39.6, 20.5. HRMS (APCI) calcd for C₁₂H₁₃O₅[M+H]⁺ 237.0763; found: 237.0790.

### (−)-ethyl (R)-2-hydroxy-4-oxo-4-phenylbutyrate (A7)

A solution of concentrated sulfuric acid (14.6 kg, 148.17 mol, 2.5 equiv) in ethanol (33.4 k, 3.0 V) was added to a solution of A6 (14.0 kg, 59.3 mol, 1.0 equiv) in ethanol (22.2 kg, 2.0 V) over 35 min with stirring at 12-14° C. (IT), heated to 20-25° C. (IT), and stirred for 16-20 h. The reaction was monitored by HPLC (cpd. A6<0.5 A %). The reaction mixture was poured to ice-water (210 kg, 1500 wt %) with stirring at 0-10° C. and the aqueous phase was extracted with i-PrOAc (123.2 kg×2, 10 V×2). The combined organic phase was washed with saturated sodium bicarbonate (72.2 kg, 5 V) and brine (72.2 kg, 5 V) The organic phase was concentrated (without drying) to remove volatiles and dried at 40° C. (ET) producing a yellow oil. The crude oil was dissolved in MTBE (10.0 kg, 1 V) with stirring and filtered at 40° C. n-Heptane (49.0 kg, 5 V) was added over 1 h at 0° C. to 5° C. (IT). The suspension was stirred at 0° C. to 5° C. (IT) for 1 h and the solids were filtered. The cake was washed with n-heptane (19.6 kg×2, 2 V×2). The white solid was dried under reduced pressure to produce the desired product (−)-ethyl (R)-2-hydroxy-4-oxo-4-phenylbutyrate A7 (10.5 kg, 83% yield) as a white solid, which is consistent with commercially available samples and literature reports (see, e.g., Li, W.; Lu, B.; Xie, X.; Zhang, Z. Org. Lett. 2019, 21, 5509-5513).

Chiral HPLC >99% ee. ¹H NMR (400 MHz, CDCl₃) δ 7.98-7.90 (m, 2H), 7.62-7.53 (m, 1H), 7.45 (dd, J=10.5, 4.8 Hz, 2H), 4.66 (dd, J=6.0, 3.9 Hz, 1H), 4.25 (q, J=7.1 Hz, 2H), 3.53 (dd, J=17.5, 3.9 Hz, 1H), 3.45 (dd, J=17.5, 6.0 Hz, 1H), 3.42 (br s, 1H), 1.26 (t, J=7.1 Hz, 3H). ¹³C NMR (101 MHz, CDCl₃) δ 197.5, 173.7, 136.4, 133.5, 128.6, 128.1, 67.2, 61.8, 42.1, 14.1. HRMS (ESI) calculated for C₁₂H₁₄NaO₄ [M+Na]⁺ 245.0790; found: 245.0786.

### tert-butyl (R,E)-2-(4-ethoxy-3-hydroxy-4-oxo-1-phenylbutylidene)hydrazine-1-carboxylate (A8)

Compound A7 (70.0 kg, 315.0 mol, 1.0 equiv) and NH₂NHBoc (54.1 kg, 409.4 mol, 1.3 equiv) in ethanol (552 kg, 10 V) and formic acid (3.6 kg, 78.2 mol, 0.25 equiv) were heated to 55-60° C. (IT) and stirred for 16 h under N₂. The reaction was monitored by HPLC (cpd. A7<2.0 A %). The reaction mixture was concentrated to remove volatiles at 40-50° C. (ET, Jacket temperature) under vacuum. Azeotropic distillation with n-heptane (93 kg×2, 2 V×2) was performed. To the suspension was added more n-heptane (186 kg, 4 V) and the mixture was stirred for 12 h at 45-50° C. The slurry was then treated with MTBE (280 kg, 5.4 V) and heptane (210 kg, 4.5 V), and was stirred further at 0° C. for additional 2 h before the solids were filtered and rinsed with n-heptane (93 kg×2, 2 V×2). The cake was dried at 40-45° C. under vacuum for 16 h to yield tert-butyl (R,E)-2-(4-ethoxy-3-hydroxy-4-oxo-1-phenylbutylidene)hydrazine-1-carboxylate A8 (95.6 kg, 92% yield) as a white solid.

HPLC: 97:3 E/Z hydrazone ratio. ¹H NMR (600 MHz, DMSO-d₆) δ 9.88 (s, 1H), 7.71-7.64 (m, 2H), 7.43-7.29 (m, 3H), 6.14 (d, J=5.4 Hz, 1H), 4.22 (dt, J=8.1, 5.0 Hz, 1H), 4.03-3.86 (m, 2H), 3.17-3.00 (m, 2H), 2.50-2.43 (m, 1H), 1.44 (s, 9H), 1.09 (t, J=7.1 Hz, 3H). ¹³C NMR (150 MHz, DMSO-d₆) δ 173.0, 153.5, 148.9, 138.0, 129.3, 128.8, 126.8, 80.1, 68.8, 61.0, 31.9, 28.6, 14.4. HRMS (ESI) calculated for C₁₇H₂₅N₂O₅ [M+H]⁺ 337.1758, found 337.1767.

### (3R,5S)-1-amino-3-hydroxy-5-phenylpyrrolidin-2-one hydrochloride (A11)

Acetic acid (675 kg, 7.5 V) was added to the suspension of TBAH (123.2 kg, 468 mol, 1.75 equiv) in CH₂Cl₂ (712 kg, 10 V) under N₂ at 10-20° C. After the addition was completed, the mixture was stirred at 20-25° C. for 1 h. To the resultant solution compound A8 (90 kg, 268 mol, 1.0 equiv) was added as a solution in CH₂Cl₂ (90 kg, 1.5 V) after being cooled to −5° C. The mixture was stirred at −5° C. (IT) for 16 h under N₂. The reaction was monitored by HPLC (cpd. A8<2.0 A %). The mixture was then quenched with ethanol (137 kg, 2 V) at 15-20° C. The resultant solution was stirred at 25-30° C. for 16 h under N₂ to afford tert-butyl 2-((1S,3R)-4-ethoxy-3-hydroxy-4-oxo-1-phenylbutyl)hydrazine-1-carboxylate A9. The reaction was monitored by HPLC (A9<0.5 A %). The reaction mixture was quenched by water (450 kg) and then extracted with CH₂Cl₂ (600 kg×2, 10 V×2). The organic phase was washed with water (450 kg×2, 5 V×2), 10% aq. sodium carbonate (500 kg, ×3, 5 V×3; pH=˜10) and brine (520 kg×2, 5 V×2; pH=˜7). The organic phase was concentrated under vacuum to produce the solution of crude tert-butyl ((3R,5S)-3-hydroxy-2-oxo-5-phenylpyrrolidin-1-yl)carbamate A10 in CH₂Cl₂.

A solution of crude compound A10 (1.0 equiv) in CH₂Cl₂ (320 kg) was cooled to 0-5° C. and then was added a solution of 6 M HCl in n-propanol (200 L, 6.0 equiv) at 60° C. The mixture was stirred at 20-25° C. for 16 h. The reaction was monitored by HPLC (cpd. A10<0.5 A %). The resultant mixture was filtered and the cake was washed with CH₂Cl₂ (200 kg×2, 2 V×2). The cake was dried in filter-dryer with N₂ flow at 40-45° C. to produce (3R,5S)-1-amino-3-hydroxy-5-phenylpyrrolidin-2-one hydrochloride A11 (45.3 kg, 85% yield, 3 steps) as a white powder.

¹H NMR (400 MHz, CD₃OD) δ 7.52-7.32 (m, 5H), 5.07 (dd, J=8.2, 4.3 Hz, 1H), 4.62 (dd, J=8.0, 6.1 Hz, 1H), 2.55 (ddd, J=14.1, 8.2, 6.1 Hz, 1H), 2.49-2.34 (m, 1H). ¹³C NMR (101 MHz, D₂O) δ 175.3, 140.1, 131.9, 131.5, 129.1, 69.1, 63.9, 38.9. HRMS (ESI) calculated for C₁₀H₁₃N₂O₂ [M+H]⁺: 193.0977; found 193.0973.

### (3R,5S)-1-amino-3-hydroxy-5-phenylpyrrolidin-2-one (A12)

The crude compound A11 (21.6 kg, 1.0 equiv) was dissolved in water (16.2 kg, 0.75 V) at 30-35° C., the solution was filtered through a polish filter (PP, 1 um). The filtrate was cooled to 10-20° C. and then was added a solution (pre-filtered through polish filtration) of sodium hydroxide (1.93 kg, 52.8 mol, 0.51 eq.) in water (2.8 kg, 0.13 V). The mixture was stirred at 10-20° C. for 60 min. Finally a solution (pre-filtered through polish filtration) of sodium hydroxide (1.93 kg, 52.8 mol, 0.51 eq.) in water (2.8 kg, 0.13 V) was added and the mixture was stirred at 10-20° C. for another 60 min. The resultant mixture was filtered and the cake was washed with cooled water (4.32 kg×1, 2.16 kg×1, 0.2 V×1, 0.1 V×1; 10-15° C.) and cooled n-PrOH (4.24 kg×2, 0.25 v x 2; 10-15° C.). The cake was dried in filter-dryer with N₂ flow at 40-45° C. to afford (3R,5S)-1-amino-3-hydroxy-5-phenylpyrrolidin-2-one A12 (13.2 kg, 73% yield) as a white powder.

HPLC: >99.5:0.5 dr. Chiral HPLC: >99.5% ee. ¹H NMR (600 MHz, DMSO-d₆) δ 7.35 (t, J=7.5 Hz, 2H), 7.28 (t, J=7.5 Hz, 1H), 7.18 (d, J=7.6 Hz, 2H), 5.69 (d, J=4.9 Hz, 1H), 4.66 (dd, J=8.4, 3.6 Hz, 1H), 4.35-4.22 (m, 1H), 2.24-2.15 (m, 1H), 2.07 (ddd, J=12.7, 8.1, 3.6 Hz, 1H). ¹³C NMR (150 MHz, DMSO-d₆) δ 171.8, 141.5, 128.6, 127.4, 126.3, 66.5, 61.4, 36.9. HRMS (ESI) calculated for C₁₀H₁₃N₂O₂ [M+H]⁺ 193.0972, found 193.0978.

### Example 3: Preparation of cyclopropyl-[(5S,7S)-7-fluoro-5-phenyl-6,7-dihydro-5H-pyrrolo[1,2-b][1,2,4]triazol-2-yl]methanone (A17)

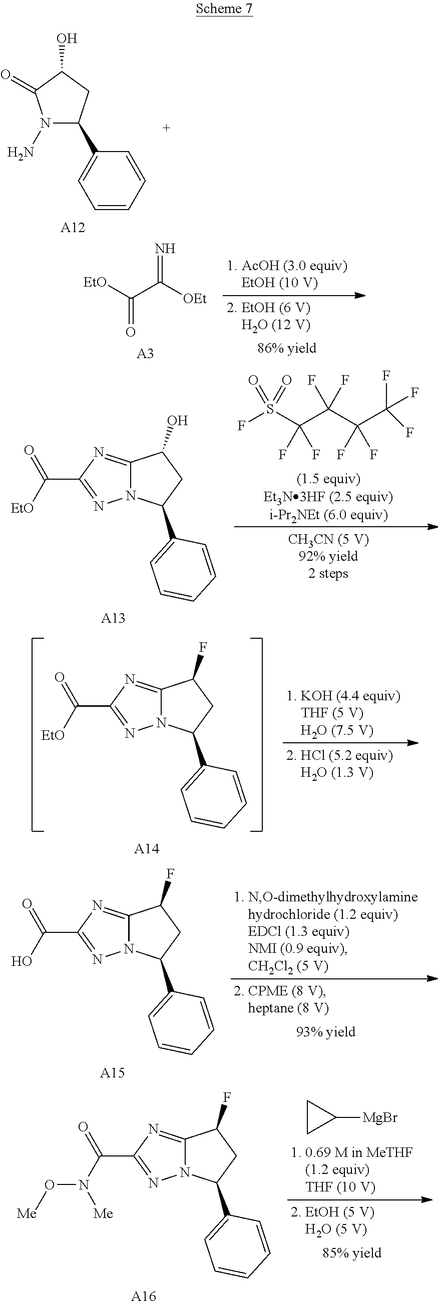

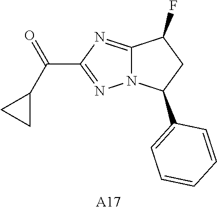 [SMILES: O=C(c1nc2n(n1)[C@H](c1ccccc1)C[C@@H]2F)C1CC1 ; "A17"]

The synthesis of cyclopropyl-[(5S,7S)-7-fluoro-5-phenyl-6,7-dihydro-5H-pyrrolo[1,2-b][1,2,4]triazol-2-yl]methanone A17 is illustrated in Scheme 7.

### Ethyl (5S,7R)-7-hydroxy-5-phenyl-6,7-dihydro-5H-pyrrolo[1,2-b][1,2,4]triazole-2-carboxylate (A13)

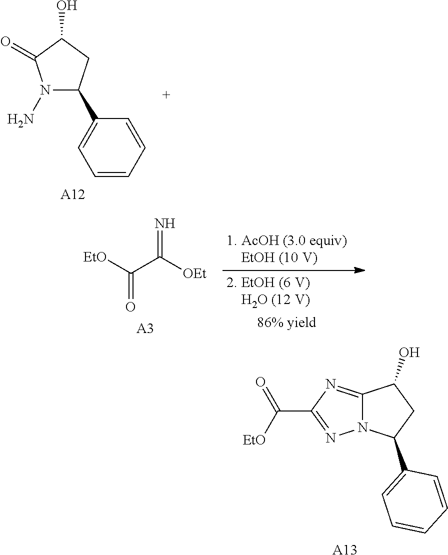

To a 100 L Reactor 1 under nitrogen was charged compound A12 (5.02 kg, 26.11 mol, 1.00 equiv). EtOH (15.95 kg, 20.22 L, 4.0 vol), compound A3 (5.60 kg, 38.58 mol, 1.47 equiv), EtOH (15.85 kg, 20.09 L, 4.0 vol), AcOH (4.60 kg, 4.38 L, 76.60 mol, 2.93 equiv), and EtOH (8.15 kg, 10.33 L, 2.1 vol) were charged into the reactor, giving a suspension. The internal temperature was adjusted to 80±10° C. and the reaction was agitated for 21 h. During this time, the reaction clarified before becoming a suspension again. The internal temperature was adjusted to 60±15° C. over 30 min. An Aurora filter was heated to a jacket temperature of 60±15° C. and a portion of Reactor 1 contents were passed through the Aurora filter, collecting ca. 70 L of filtrate in a clean 100 L Reactor 2. An insoluble solid that is insoluble in certain organic solvents is retained. For example, the solid is an oligomer or polymer of compound A3 (ethyl-2-ethoxy-2-iminoacetate) confirmed by solid state NMR spectroscopic analysis.

The first portion of filtrate in Reactor 2 was concentrated under reduced pressure over 1 h to a volume of ca. 55 L (11 vol), maintaining internal temperature below 60° C. EtOH (16.30 kg, 20.66 L, 3.2 vol) was added to Reactor 1 and the remaining Reactor 1 contents were passed through the Aurora filter, collecting an additional ca. 20 L of filtrate in Reactor 2 to give a total volume of ca. 75 L. The contents of Reactor 2 were concentrated under reduced pressure over 1 h to a volume of ca. 30 L (6 vol), maintaining internal temperature below 60° C. Solid formation may be observed as the solution approaches low volume. Before the addition of seeds, a suspension may be observed. In some batches, the premature solid formation events appear to not affect the quality of the product. The reactor was cooled to an internal temperature of 25±10° C. Water (15.10 kg, 15.10 L, 3.0 vol) was added to Reactor 2 over 60±30 min. Compound A13 seeds (25 g, 0.5 wt %) and water (150 mL) were combined in a glass bottle and charged into Reactor 2. A suspension was observed. Additional water (49.15 kg, 49.15 L, 9.8 vol) was added to Reactor 2 over 60±30 min. Reactor 2 was agitated for 3 h. The internal temperature was adjusted to 0±5° C. over 3 h and agitated for a 10 h. The slurry was transferred to the filter dryer, collecting the filtrate in glass carboys.

Reactor 2 was rinsed with water (23.30 kg, 23.30 L, 4.6 vol), agitated for a minimum of 5 min, and the contents were transferred to the filter dryer, collecting the filtrates in glass carboys. The solid cake in the filter dryer was rinsed with water (20.55 kg, 20.55 L, 4.1 vol). Minimization of AcOH in solid sample is, for example, needed to prevent competition with fluoride nucleophile in alcohol in the subsequent deoxyfluorination step. In some examples, high levels of acetic acid have led to the formation of acetate product.

The solid cake was dried under vacuum with nitrogen sweep at a jacket temperature of 50±5° C. for 27 h with intermittent agitation. The filter dryer was cooled to a jacket temperature of 20±5° C. and the product compound A13 (6.36 kg, 23.27 mol, 86% yield), a crystalline off-white solid, was discharged into a sealed bag.

HPLC 99.9 A %, >99.9:0.1 dr. ¹H NMR (600 MHz, DMSO-d₆) δ 7.44-7.32 (m, 3H), 7.25 (d, J=7.3 Hz, 2H), 6.18 (d, J=5.9 Hz, 1H), 5.76 (t, J=6.6 Hz, 1H), 5.33-5.25 (m, 1H), 4.37-4.22 (m, 1H), 2.99-2.86 (m, J=6.3 Hz, 2H), 1.27 (t, J=7.1 Hz, 3H). ¹³C NMR (150 MHz, DMSO-d₆) δ 163.6, 159.5, 158.8, 138.6, 128.9, 128.5, 127.0, 62.6, 61.0, 60.2, 46.5, 14.0. HRMS (ESI) calculated for C₁₄H₁₆N₃O₃ [M+H]⁺ 274.1186, found 274.1195.

Other acid additives were evaluated, including, for example, oxalic acid, succinic acid, benzoic acid, isobutyric acid, pivalic acid, salicylic acid, oxamic acid, 2-picolinic acid, trifluoroacetic acid, p-toluenesulfonic acid, methanesulfonic acid, formic acid, hydrochloric acid in ethanol, trimethylsilyl chloride in ethanol. In some examples, yields for the other acid additives fell within the range of 14-81%. In some examples, the yield is 98% wherein the acid additive is acetic acid.

Other reagents replacing compound A3 were evaluated, including, for example, ethyl thiooxamate, ethyl cyanoformate, methyl cyanoformate and triethyl 1,3,5-triazine-2,4,6-tricarboxylate. In some examples, HLPC conversion rates and yields for these reagents fell within the range of 90-100% and 40-76%, respectively. In other examples, the yields for these reagents resulted in yields less than 81%.

In one example, the acid additive is isobutyric acid and the reagent reacting with compound A12 is ethyl thiooxamate resulting in a yield of 72.3%. In another example, the acid additive is salicylic acid and the reagent is triethyl 1,3,5-triazine-2,4,6-tricarboxylate resulting in a yield of 81%. In yet another example, the acid additive is formic acid and the reagent is ethyl cyanoformate resulting in a yield of 14%.

### (5S,7S)-7-fluoro-5-phenyl-6,7-dihydro-5H-pyrrolo[1,2-b][1,2,4]triazole-2-carboxylic acid (A15)

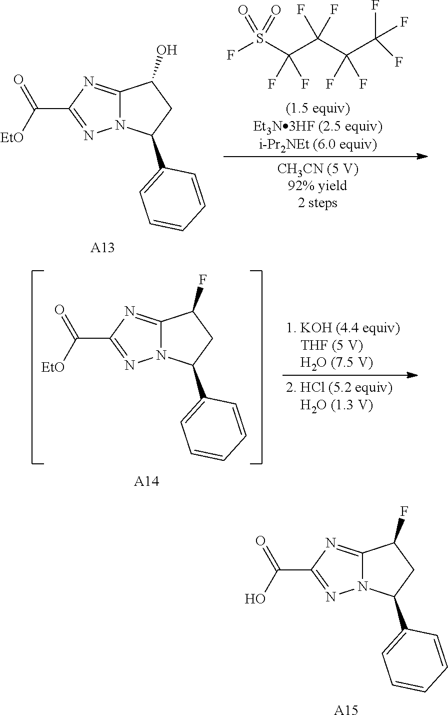

To a 100 L Reactor 1 under nitrogen was charged compound A13 (4.51 kg, 15.97 mol, 1.00 equiv), CH₃CN (17.90 kg, 22.77 L, 5.0 vol), and i-Pr₂NEt (12.95 kg, 100.20 mmol, 6.27 equiv). Et₃N·3HF (5.30 kg, 32.88 mmol, 2.59 equiv) was added slowly over 1 h by peristaltic pump, maintaining internal temperature below 30° C. with jacket cooling (addition of Et₃N·3HF is exothermic). CH₃CN (0.36 kg, 0.45 L, 0.1 vol) was added through the same peristaltic pump tubing. PBSF (7.50 kg, 24.83 mmol, 1.55 equiv) was added slowly over 1 h, maintaining temperature below 30° C. with jacket cooling (addition of PBSF is exothermic). CH₃CN (0.36 kg, 0.45 L, 0.1 vol) was added through the same peristaltic pump tubing. The contents of Reactor 1 were agitated for a 1 h.

2-MeTHF (31.00 kg, 36.05 L, 8.0 vol) was added to Reactor 1 and the mixture was stirred for 20 min. The contents of Reactor 1 were transferred to a 200 L Reactor 2 containing a stirring solution of K₂HPO₄ (7.88 kg) in water (32.40 kg, 32.40 L, 7.2 vol), maintaining internal temperature below 30° C. with jacket cooling. 2-MeTHF (17.40 kg, 20.23 L, 4.5 vol) was added to Reactor 1 and the rinse was transferred to Reactor 2, maintaining internal temperature below 30° C. with jacket cooling. Agitation was halted and the layers were allowed to settle for 30 min. The triphasic mixture was separated with the two lower layers collected into separate glass carboys (the lowest layer is referred to as the dense layer and may contain fluorous byproducts while the middle layer is referred to as the aqueous layer). A solution of NaCl (14.49 kg) in water (63.35 kg, 63.35 L, 14.0 vol) was prepared. A portion of this NaCl solution (25.72 kg) was transferred to Reactor 2 and the contents were stirred for a minimum of 5 min. Agitation was halted and the layers were allowed to settle for at least 30 min (actual time: 30 min).

The biphasic mixture was separated and the aqueous phase was collected into a glass carboy. Another portion of the NaCl solution (25.72 kg) was transferred to Reactor 2 and the contents were stirred for a 10 min. Agitation was halted and the layers were allowed to settle for 15 min. The biphasic mixture was separated and the aqueous phase was collected into a glass carboy. Another portion of the NaCl solution (25.72 kg) was transferred to Reactor 2 and the contents were stirred for a minimum of 5 min. Agitation was halted and the layers were allowed to settle for 30 min. The biphasic mixture was separated and the aqueous phase was collected into a glass carboy. A crude solution of compound A14 in 2-MeTHF and CH₃CN was obtained.

A portion of the solution in Reactor 2 (ca. 60 L) was transferred to a clean 100 L Reactor 3. The contents of Reactor 3 were distilled under reduced pressure to ca. 30 L (6.7 vol), maintaining internal temperature below 50° C. THF (120.15 L, 135.15 L, 29.9 vol) was added to Reactor 2. The contents of Reactor 2 were transferred to Reactor 3 continuously to maintain target volume of 27-45 L (6.0-10.0 vol). The distillation proceeded over 4 h, was halted for 16 h, and resumed for 1 h, reaching a final volume of ca. 40 L (8.9 vol). Distillation was continued until a volume of ca. 23 L (5.1 vol) was achieved. The solution was cooled to an internal temperature of 35±10° C. Reduction of 2-MeTHF and CH₃CN content may facilitate the ester hydrolysis.

Reactor 3 was cooled to an internal temperature of 20±10° C. and water (22.85 kg, 22.85 L, 5.1 vol) was added followed by a solution of KOH (3.25 kg, 57.92 mmol, 4.38 equiv) in water (11.90 kg, 11.90 L, 2.6 vol) (5 M aq KOH), maintaining temperature below 30° C. The reaction mixture was stirred for 1 h. Water (22.90 kg, 22.90 L, 5.1 vol) was charged to Reactor 3 and the contents were transferred to Reactor 2. A solution of conc HCl (6.75 kg) in water (5.80 kg, 5.80 L, 1.3 vol) (6 M aq HCl) was added slowly, maintaining internal temperature below 30° C. while vapors are passed through a NaOH scrubber solution with phenolphthalein indicator. Smoky vapor may be HCl, which would be neutralized by the NaOH scrubber if it is formed. The reaction was stirred for 13 h at 20±10° C. The pH range (e.g., target: 0≤pH≤2) ensures, for example, full protonation to the carboxylic acid and minimal loss in aqueous washes during filtration.

The suspension was transferred to the Filter Dryer, collecting the filtrate in glass carboys. The filter cake was rinsed with a solution of water (40.30 kg, 40.30 L, 8.9 vol) and THF (3.85 kg, 4.33 L, 1.0 vol), collecting the filtrate in a glass carboy. The mixed organic/aqueous wash is used to purge residual perfluorobutanesulfonate salts that may still be present after the acidification. For example, use tests that did not employ this wash showed extra fluorine signals in ¹⁹F NMR analysis of the isolated material. The filter cake was rinsed with water (45.12 kg, 45.12 L, 10 vol), collecting the filtrate in a glass carboy. The jacket temperature was increased to 50±5° C. and the wet cake was dried for 4 days. The filter dryer was cooled to a jacket temperature of 20±5° C. and the product compound A15 (3.76 kg, 14.66 mol, 92% yield), a pale tan solid, was discharged into a sealed bag.

HPLC 99.8 A %, >99.95:0.05 dr. ¹H NMR (600 MHz, DMSO-d₆) δ 7.45-7.40 (m, 2H), 7.40-7.35 (m, 1H), 7.24 (d, J=7.5 Hz, 2H), 6.21 (ddd, J=56.6, 7.2, 1.9 Hz, 1H), 5.69 (ddd, J=9.1, 6.7, 3.0 Hz, 1H), 3.72 (ddd, J=25.8, 15.4, 7.8, 7.8 Hz, 1H), 2.70 (ddd, J=26.8, 15.3, 2.4, 2.4 Hz, 1H). ¹³C NMR (150 MHz, DMSO-d₆) δ 160.5, 158.8, 138.4, 129.0, 128.6, 126.7, 126.7, 83.2, 60.0, 42.9. ¹⁹F NMR (565 MHz, DMSO-d₆) δ −167.0. HRMS (ESI) calculated for C₁₂H₁₁FN₃O₂[M+H]⁺ 248.0830, found 248.0837.

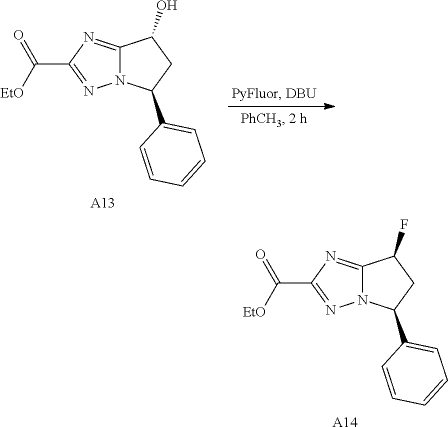 [SMILES: CCOC(=O)c1nc2n(n1)[C@H](c1ccccc1)C[C@H]2O ; "A13" ; CCOC(=O)c1nc2n(n1)[C@H](c1ccccc1)C[C@@H]2F ; "A14"]

In another example, toluene (70 mL, 10 vol.), DBU (7 g, 0.11 mol, 2.0 eq.) and compound A12 (7 g, 0.11 mol, 1.0 eq.) were charged into a 250 mL flask at r.t. (˜ 24° C.) under N₂ atmosphere. The solution was cooled to 0-10° C. PyFluor (7 g, 0.11 mol, 1.2 eq.) was added drop-wise into above solution at 0-10° C. The solution was stirred for 2 h at 0-10° C. Upon reaction completion, the mixture was quenched with saturated aqueous NH₄Cl at 0-20° C. The mixture was extracted with TBME (70 mL, 10 vol.) The organic phase was washed with brine (70 mL, 10 vol.) and then dried with Na₂SO₄ (˜5 g). The mixture was filtered and the cake was washed with TBME (20 mL). The filtrate was concentrated under vacuum at 35° C. to provide about 10 g of crude compound A14 with 95 A % HPLC purity in quantitative yield.

### (5S,7S)-7-fluoro-N-methoxy-N-methyl-5-phenyl-6,7-dihydro-5H-pyrrolo[1,2-b][1,2,4]triazole-2-carboxamide (A16)

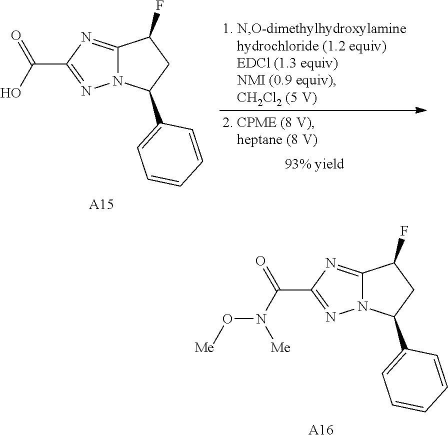 [SMILES: O=C(O)c1nc2n(n1)[C@H](c1ccccc1)C[C@@H]2F ; "A15" ; CON(C)C(=O)c1nc2n(n1)[C@H](c1ccccc1)C[C@@H]2F ; "A16"]

To a 100 L Reactor 1 under nitrogen was charged compound A15 (4.22 kg, 17.03 mol, 1.00 equiv), N,O-dimethylhydroxylamine HCl (2.09 kg, 21.00 mol, 1.23 equiv), CH₂Cl₂ (16.80 kg, 12.68 L, 3.0 vol), 1-methylimidazole (1.25 kg, 15.22 mol, 0.89 equiv), CH₂Cl₂ (2.85 kg, 2.15 L, 0.5 vol), EDCI (4.36 kg, 21.83 mol, 1.28 equiv), and CH₂Cl₂ (8.75 kg, 6.60 L, 1.6 vol). Process optimization found that reduced charge of 1-methyl imidazole can lead to cleaner reaction profile and faster reaction rate and reduced charge of EDCI (1.5 equiv in telescoped Step 4/5 procedure vs 1.3 equiv in this modified procedure) may not have a negative impact on the reaction. The contents of Reactor 1 were agitated for 2 h.

A 6 M aq HCl solution was prepared from conc HCl (6.34 kg) in water (15.75 kg, 15.75 L). This 6 M aq HCl solution (21.17 kg) was charged into Reactor 1 slowly while maintaining internal temperature below 30° C. The contents of Reactor 1 were stirred for 10 min. Agitation was halted and the phases were allowed to settle for 15 min. The aqueous phase was discharged into a glass carboy and the organic phase was transferred back into Reactor 1. A solution of K₂HPO₄ (2.11 kg) in water (19.01 kg, 19.01 L) was prepared. A portion of this aq K₂HPO₄ solution (21.13 kg) was transferred into Reactor 1, maintaining an internal temperature below 30° C. The contents of Reactor 1 were stirred for 10 min. Agitation was halted and the phases were allowed to settle for 15 min. The aqueous phase was discharged into a glass carboy and the organic phase was transferred back into Reactor 1. Water (21.13 kg, 21.13 L, 5.0 vol) was charged into Reactor 1. Agitation was halted and the phases were allowed to settle for 15 min.

The aqueous phase was discharged into a glass carboy and the organic phase was transferred back into Reactor 1. The contents of Reactor 1 were distilled to ca. 12 L (2.8 vol) volume, maintaining temperature below 50° C. CPME (18.20 kg, 21.16 L, 5.0 vol) was added to Reactor 1. From an HTE solubility screen, CPME was, for example, found to be a unique solvent that provided high solubility at high temperature and low solubility at low temperature. The contents of Reactor 1 were distilled under reduced pressure to a target volume of ca. 25 L (5.9 vol), maintaining temperature below 65° C. CPME (7.27 kg, 8.45 L, 2.0 vol) was added to Reactor 1. The contents of Reactor 1 were distilled under reduced pressure to a target volume of ca. 33 L (7.8 vol), maintaining temperature below 65° C.

The temperature of the reactor was adjusted to 80±5° C. The reaction was still a solution. The temperature of the reactor was adjusted to 60±5° C. over 40 min. A slurry of (5S,7S)-7-fluoro-N-methoxy-N-methyl-5-phenyl-6,7-dihydro-5H-pyrrolo[1,2-b][1,2,4]triazole-2-carboxamide A16 seeds (20.5 g, 0.071 mmol, 0.5 wt %) in CPME (84.5 g, 98 mL) was charged into Reactor 1. The reaction began to form a suspension. The reactor contents were agitated for 30 min. Heptane (8.66 kg, 12.66 L, 3.0 vol) was added to Reactor 1 over 30 min. Heptane was, for example, added as anti-solvent to further reduce the mother liquor loss in the crystallization. Reactor 1 was adjusted to 0±5° C. over 3 h. The reactor contents were agitated for 16 h. Heptane (14.45 kg, 21.13 L, 5.0 vol) was charged to Reactor 3, cooled to 0° C., and stirred for 4.5 h. The contents of Reactor 1 were transferred to a filter dryer maintained at 20° C., collecting the filtrates in Reactor 1. The contents of Reactor 1 were transferred to the filter dryer, and the filtrates were collected in Reactor 1 once again.

The contents of Reactor 3 were transferred to the filter dryer, collecting the filtrates in Reactor 1. The contents of the filter dryer were dried under vacuum with a nitrogen sweep at ambient temperature for 21 h. The filter dryer jacket temperature was increased to 50±5° C. and the contents were dried for 3 days with intermittent agitation of the wet cake. The filter dryer was adjusted to a temperature of 20±10° C. and the product (5S,7S)-7-fluoro-N-methoxy-N-methyl-5-phenyl-6,7-dihydro-5H-pyrrolo[1,2-b][1,2,4]triazole-2-carboxamide A16 (4.62 kg, 15.88 mol, 93% yield), a pale tan solid, was discharged into a sealed bag.

HPLC 99.8 A %, >99.95:0.05 dr. ¹H NMR (600 MHz, DMSO-d₆) δ 7.47-7.32 (m, J=7.7, 6.5, 1.4 Hz, 3H), 7.26-7.19 (m, 2H), 6.21 (ddd, J=56.7, 7.1, 1.8 Hz, 1H), 5.74-5.65 (m, 1H), 3.83-3.65 (m, 1H), 3.69 (s, 3H), 3.30 (s, 3H), 2.78-2.62 (m, 1H). ¹³C NMR (150 MHz, DMSO-d₆) δ 161.9, 158.0, 138.5, 129.0, 128.6, 126.5, 99.5, 83.2, 61.6, 59.8, 43.1, 32.3. ¹⁹F NMR (565 MHz, DMSO-d₆) δ −166.6. HRMS (ESI) calculated for C₁₄H₁₆FN₄O₂[M+H]⁺ 291.1252, found 291.1265.

### Cyclopropyl-[(5S,7S)-7-fluoro-5-phenyl-6,7-dihydro-5H-pyrrolo[1,2-b][1,2,4]triazol-2-yl]methanone (A17)

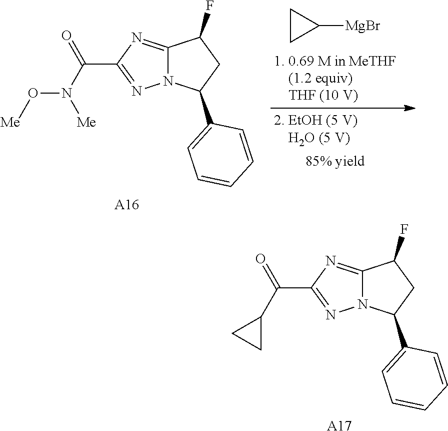

To a 100 L Reactor 1 under nitrogen was charged compound A16 (4.52 kg, 15.54 mol, 1.00 equiv) and THF (47.95 kg, 10.6 vol). Reactor 1 was adjusted to an internal temperature of −5° C.±10° C. Cyclopropylmagnesium bromide solution (0.69 M in 2-MeTHF) (27.84 kg, 18.80 mol, 1.21 equiv) was charged slowly into Reactor 1 over 2 h, maintaining an internal temperature of −5° C. 10° C., and was titrated with 2-butanol titration according to the Sigma Aldrich Quality Control SOP. Titration value of Sigma Aldrich commercial Grignard solution declined from 0.89 M to 0.69 M over 3 months. The contents of Reactor 1 were agitated at −5° C.±10° C. for 30 min.

A 6 M aq HCl solution was prepared from conc HCl (6.55 kg) in water (16.94 kg, 17.10 L). A portion of the previously prepared 6 M aq HCl solution (23.55 kg) and 2-MeTHF (38.15 kg, 44.3 L, 9.8 vol) was charged into Reactor 2. Reactor 2 was cooled to −15° C. The contents of Reactor 1 were transferred slowly to Reactor 2 while maintaining internal temperature of Reactor 2 below 10° C. The quench is exothermic and slow transfer is advised. 2-MeTHF (4.29 kg, 4.98 L, 1.1 vol) was charged into Reactor 1 to rinse and transfer to Reactor 2, maintaining internal temperature of Reactor 2 below 10° C. The temperature of Reactor 2 was adjusted to 20±5° C. The contents of Reactor 2 were stirred 15 min. Agitation was halted and the phases were allowed to settle for 30 min.

The aqueous phase was discharged into a glass carboy. A solution of K₂HPO₄ (2.24 kg) in water (20.33 kg, 20.33 L) was prepared. A portion of the previous aq K₂HPO₄ solution (22.55 kg) was transferred into Reactor 2. The contents of Reactor 2 were stirred for a minimum of 10 min. Agitation was halted and the phases were allowed to settle for 10 min. The aqueous phase was discharged into a glass carboy. A solution of NaCl (1.19 kg) in water (21.46 kg, 21.45 L) was charged into Reactor 2. The contents of Reactor 2 were stirred for 10 min. Agitation was halted and the phases were allowed to settle for 30 min. The aqueous phase was discharged into a glass carboy.

The contents of Reactor 2 were pumped through a carbon cartridge in Graver Filter Housing and polish filtered into a glass carboy over 2.0±1.5 h (actual time: 2 h). Reactor 2 was charged with THF (32.55 kg, 36.61 L, 8.1 vol) and the contents of Reactor 2 were pumped through a Graver C941 carbon cartridge in Graver Filter Housing and polish filtered into a glass carboy over 60 min 55 min (actual time: 1 h, report (HPLC): cpd. A17% wt/wt: 11.46%).

The contents of the glass carboy were charged into Reactor 1 and distilled under reduced pressure to a target volume of 17 L (3.8 vol). EtOH (17.05 kg, 21.61 L, 4.8 vol) was passed through a polish filter and charged into Reactor 1. The contents of Reactor 1 were distilled under reduced pressure to a target volume of ca. 20 L (4.4 vol). EtOH (17.05 kg, 21.61 L, 4.8 vol) was passed through a polish filter and charged into Reactor 1. The contents of Reactor 1 were distilled under reduced pressure to a target volume of ca. 21 L (4.6 vol).

Water (4.00 kg, 4.00 L, 0.9 vol) was passed through a polish filter and charged into Reactor 1. Reactor 1 was adjusted to a temperature of 65±5° C. over 20 min. A slurry of compound A17 seeds (19.9 g, 0.073 mmol, 0.4 wt %) in polish-filtered water (201 g, 201 mL) and polish-filtered EtOH (156 g, 198 mL) was charged into Reactor 1. The contents of Reactor 1 began to form a suspension. Reactor 1 was agitated at 60±5° C. for 20 min. Water (15.65 kg, 15.65 L, 3.5 vol) was passed through a polish filter and charged into Reactor 1, maintaining temperature above 55° C. Reactor 1 was agitated at 60±5° C. for 30 min. The temperature was adjusted to 0±5° C. over 4.5 h. The contents of Reactor 1 were stirred at 0±5° C. for 8.5 h.

An IKA Magic Lab Mill was equipped with Dispax Reactor DR Module and 2G4M6F Stator/Rotor. The IKA Magic Lab Mill was cooled to 0±5° C. and set to 26000 RPM milling speed. The contents of Reactor 1 were passed through the IKA Magic Lab Mill into the filter dryer, collecting the filtrate in Reactor 3. Polish-filtered EtOH (12.65 kg, 16.03 L, 3.5 vol) and polish-filtered water (15.85 kg, 15.85 L, 3.5 vol) were charged into a glass carboy to prepare the cake wash solution. The cake wash solution was charge into Reactor 1 and temperature of Reactor 1 was adjusted to 5° C. The contents were stirred for 30 min and the remaining contents of Reactor 1 were passed through the IKA Magic Lab Mill into the filter dryer, collecting the filtrate in Reactor 3. The contents of the filter dryer were dried under vacuum with nitrogen sweep at ambient temperature for 19 h. The filter dryer was adjusted to 40±5° C. and the contents were dried for 4 days.

The filter dryer was adjusted to a temperature of 20±10° C. and the product cyclopropyl-[(5 S,7S)-7-fluoro-5-phenyl-6,7-dihydro-5H-pyrrolo[1,2-b][1,2,4]triazol-2-yl]methanone A17 (3.54 kg, 13.17 mol, 85% yield), a pale tan-orange solid, was discharged into a sealed bag.

HPLC 99.1 A %, >99.95:0.05 dr. Chiral HPLC >99.9% ee. ¹H NMR (600 MHz, DMSO-d₆) δ 7.48-7.35 (m, 3H), 7.33-7.21 (m, 2H), 6.24 (ddd, J=56.4, 7.2, 2.0 Hz, 1H), 5.73 (ddd, J=8.5, 6.5, 3.1 Hz, 1H), 3.75 (dddd, J=25.7, 15.6, 8.6, 7.2 Hz, 1H), 3.04-2.95 (m, 1H), 2.72 (dddd, J=26.8, 15.2, 3.2, 2.0 Hz, 1H), 1.14-0.99 (m, 4H). ¹³C NMR (150 MHz, DMSO-d₆) δ 192.7, 165.6, 159.1, 138.3, 129.1, 128.7, 126.7, 83.2, 60.0, 43.0, 17.9, 11.9, 11.8. ¹⁹F NMR (565 MHz, DMSO-d₆) δ −167.5. HRMS (ESI) calculated for C₁₅H₁₅FN₃O [M+H]⁺ 272.1197, found 272.1202.

### Example 4: Preparation of (3R,5S)-1-amino-3-hyroxy-5-phenylpyrrolidin-2-one A12

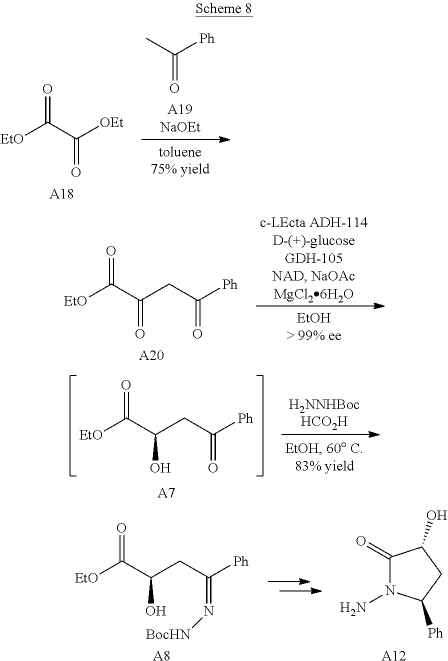

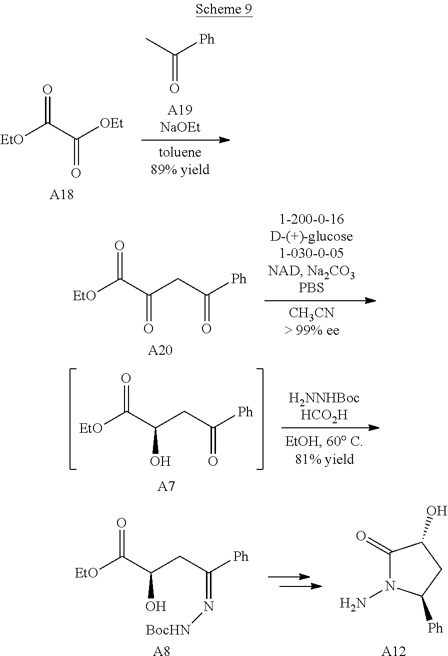

The synthesis of (3R,5S)-1-amino-3-hyroxy-5-phenylpyrrolidin-2-one A12 is illustrated in Schemes 8 and 9.

### Ethyl 2,4-dioxo-4-phenyl-butanoate (A20)

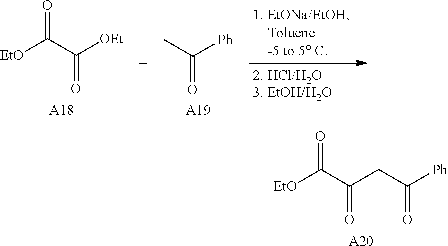

To a glass reactor was charged toluene (80.1 kg, 10 wt./wt., 11.9 vol.) and 20% EtONa/EtOH solution (31.5 kg, 92.6 mol, 1.4 eq.) at 20-30° C. The contents were cycled from vacuum to nitrogen three times then were cooled to −5 to 5° C. To the mixture was added A18 (10.1 kg, 69.1 mol, 1.1 eq.) dropwise at 0-10° C. After the addition was complete, the mixture was stirred for 10 min followed by addition of a solution of A19 (7.7 kg, 64.1 mol, 1.0 eq.) in toluene (24.1 kg, 3.1 wt./wt., 3.56 vol.). After the addition was complete, the mixture was stirred at −5 to 5° C. for 2 h. The pH of the quenching mixture was adjusted to 2-4 with 2 M HCl (48.2 kg, 6.3 wt./wt., 6.2 vol.), and adjust the pH of the mixture to 4-5. Separate the bi-phase. Wash the organic layer with 10% NaCl aq. twice (each with 24.0 kg, 3.1 wt./wt., 4.0 vol.). Concentrate the organic layer to (7.7-15.4 L) 1-2 vol. Charge (32.0 kg, 4.15 wt/wt., 5 vol) EtOH into the concentrated mixture and then concentrated to (7.7-15.4 L) 1-2 vol. Charge (15.2 kg, 2.4 vol., 2.0 wt./wt.) EtOH into the mixture. Stir the mixture at 5-15° C. Add (0.16 kg, 0.01 wt./wt.) A18 seeds. Add (12.8 kg, 1.7 wt./wt., 1.7 vol.) water in 6 h. After adding, warm the mixture to 35-45° C. Stir the mixture at 35-45° C. for 0.5 h. Then cool down the mixture to 0-5° C. over 5 h. Stir the mixture at 0-5° C. for 5 h. Filter and wash with EtOH/H₂O (1/5) (15.0 kg, 2.0 wt./wt., 2.4 vol.). Dry the wet cake at 25-30° C. for 10-30 h. Obtain the yellow solid (12.6 kg, 1.6 wt./wt., 57.3 mol, 89.4% yield, 99.5% HPLC purity, 98.3% HPLC assay).

### (−)-ethyl (R)-2-hydroxy-4-oxo-4-phenylbutyrate (A7)

Alternative 1:

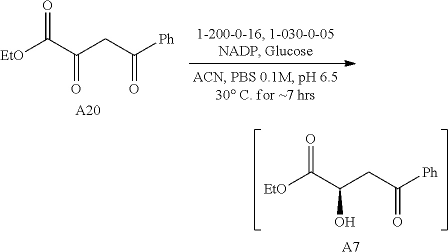

To a 300 L stir tank was charged Na₂CO₃ (6.8 kg, 0.54 w/w, 1.12 eq.) and H₂O (68.0 kg, 5.4 w/w, 5.4 vol.) and stirred to dissolved at room temperature. Barreled and labeled as 9% Na₂CO₃ solution. To the 300 L stir tank was charged Na₂CO₃ (10.1 kg, 0.80 w/w, 1.66 eq.) and H₂O (50.4 kg, 4.0 w/w, 4.0 vol.) and stirred to dissolve at room temperature. Barreled and labeled as 16.7% Na₂CO₃ solution. Washed 300 L stir tank to neutral. To the washed 300 L stir tank was charged HCl (18.0 kg, 1.43 w/w, 3.18 eq.) and H₂O (15.4 kg, 1.22 w/w, 1.22 vol.) and stirred to dissolve at room temperature. Barreled and labeled as 6 M HCl solution. To a 500 L stir tank was charged A18 (12.6 kg, 1 w/w, 57.3 mol, 1.00 eq.) and ACN (2.6 kg, 0.21 w/w, 0.26 vol.) and stirred to dissolved at room temperature.

To a 50 L preparation container was charged K₂HPO₄ (0.4 kg, 0.032 w/w, 0.04 eq.), KH₂PO₄ (0.6 kg, 0.048 w/w, 0.077 eq.), glucose (16.8 kg, 1.33 w/w, 1.63 eq.) and H₂O (50.5 kg, 4.0 w/w, 4 vol.) and stirred to dissolved at room temperature. Barreled and labeled as glycosylated buffer solution. Transferred to 500 L stir tank at 25-30° C. (recommend: 28° C.). Washed the container with H₂O (6.2 kg, 0.49 w/w, 0.49 vol.) and then transferred to the 500 L stir tank. Stirred at 25˜30° C. and adjusted pH to 6.0-6.5 by titrating 9% Na₂CO₃. To a 50 L preparation container was charged NADP (0.069 kg, 0.0054 w/w, 0.0015 eq.), KRED 1-200-0-16 (0.284 kg, 0.023 w/w), coenzyme 1-030-0-05 (0.035 kg, 0.0028 w/w) and H₂O (3.33 kg, 0.26 w/w, 0.26 vol.) and stirred to dissolved at room temperature. Then transferred to 500 L stir tank. Washed the container with H₂O (5.0 kg, 0.40 w/w, 0.4 vol.) and then transferred to 300 L stir tank. Stirred at 25-30° C. (recommend: 28° C.) and maintained pH between 6.0-6.5 (recommend: pH=6.3) by titrating 9% Na₂CO₃ (37.5 kg, ˜2.97 vol.) for 7 h.

Adjusted the pH to 1.5˜2.0 (recommend: pH=1.7) by 6 M HCl solution (12.2 kg, ˜0.97 vol). Stirred for 1 h. Then adjusted the pH back to 6.0˜6.5 by 16.7% Na₂CO₃ solution (22.6 kg, 1.79 w/w, ˜1.8 vol.). To a 100 L filter tank was charged celite (2.1 kg, 0.17 w/w). Filtered the reaction slurry to obtained filtrate 1 and cake 1. Transferred filtrate 1 to 500 L stir tank and cake 1 to 500 L stir tank. To 500 L stir tank was charged MTBE (93.6 kg, 7.43 w/w) and stirred for 25-35 min. (Recommend: 30 min) at 25˜30° C. Filtered the slurry to obtained filtrate 2 and cake 2. Transferred filtrate 2 to 500 L stir tank to extract filtrate 1. After phase separated, transferred the upper organic phase 1 to transit drums and the bottom aqueous phase 1 to 500 L stir tank. Transferred cake 2 to 300 L stir tank. To 500 L stir tank was charged MTBE (93.2 kg, 7.40 w/w, 10 vol.) and stirred for 25-35 min. (Recommend: 30 min). Filtered the slurry to obtained filtrate 3 and cake 3. Discard cake 3. Transferred filtrate 3 to 500 L stir tank to extract aqueous phase 1. After phase separated, transferred the upper organic phase 2 to transit drums and the bottom aqueous phase 2 to 500 L stir tank.

To 500 L stir tank was charged MTBE (93.1 kg, 7.39 w/w, 10 vol.) and stirred for 25-35 min. (Recommend: 30 min). Filtered the slurry to obtained filtrate 4 and cake 4. Transferred filtrate 4 to 500 L stir tank to extract aqueous phase 2. After phase separated, transferred the upper organic phase 3 to transit drums and discarded the bottom aqueous phase 3. Combined organic phase 1, 2, and 3 and charged to 500 L stir tank. Concentrated at −0.06 MPa to −0.10 MPa and 30-40° C. (optimal 40° C.) to 3-4V (37.8-57.4 L). Ethanol (˜59 kg*3, ˜4.7 V*3) was charged to 500 L stir tank and then concentrated to 3-4 V (37.8-50.4 L) three times to switch solvent. Obtained A7 at purity >95.0%, chiral purity >99.0% (HPLC 97 A %, Chiral HPLC >99% ee). The obtained solution was used directly in the next step.

Alternative 2:

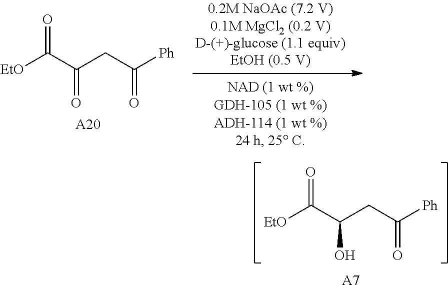

A 30 L reactor with overhead stirring, pH probe, peristaltic pump, jacket temperature 20-25° C. was charged with 0.2 M aqueous sodium acetate pH 7 (3.76 kg) followed by D-(+)-glucose (1.00 kg, 5064 mmol, 1.10 equiv). The reactor was rinsed with additional 0.2 M aqueous sodium acetate (3.76 kg) followed by 0.1 M aqueous magnesium chloride (0.20 kg) The contents of reactor 1 were stirred until dissolution was achieved (˜10 min). Next A20 (1.00 kg, 90.3 wt %, 4100 mmol, 1.0 equiv) was charged as a solid, followed by ethanol (0.4 kg) and the pH probe was set to continuously adjust the pH to 7 using 1 N aqueous sodium hydroxide (ultimately 0.19 kg was consumed). NAD (0.01 kg, 15.0 mmol, 1 wt %) was charged as solid, followed by a solution of GDH-105 (0.01 kg, 1 wt %) in 0.2 M aqueous sodium acetate pH 7 (0.2 kg) and a solution of ADH-114 (0.01 kg, 1 wt %) in 0.2 M in sodium acetate pH 7.0 (0.2 kg). The reaction was stirred at Tᵢ=25° C. for 24 h (at 24 h: >95 A % A7).

At the end of the reaction, the pH control was stopped and 6 N aqueous hydrochloric acid (1.03 kg was added to the reaction mixture until pH<2 was achieved and stirred for 1 h. MTBE (7.37 kg) was then added and the reaction was stirred vigorously for an additional 30 min. The layers were separated and the aqueous layer was back-extracted 2 more times with MTBE (7.44 kg and 3.83 kg). The organic layers were then combined in a separate 30 L Reactor, and concentrated to 8 V by distillation (260 mbar pressure, Tⱼ 60-70° C., Tᵢ 40-45° C.). Then ethanol (9.32 kg) was added and the reaction was concentrated to 8 V. Obtained A7 at purity ˜95%, chiral purity >99.0% (HPLC 95 A %, Chiral HPLC >99% ee). The obtained solution was used directly in the next step.

### tert-butyl (R,E)-2-(4-ethoxy-3-hydroxy-4-oxo-1-phenylbutylidene)hydrazine-1-carboxylate (A8)

Alternative 1:

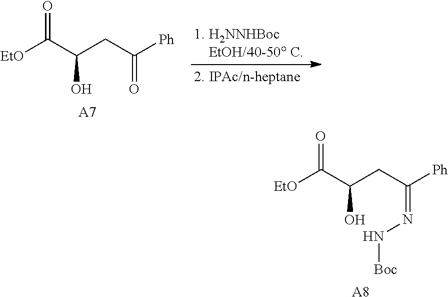

To a glass reactor was charged EtOH (97.4 kg, 10 vol, 7.9 wt/wt) and A7 (12.3 kg, 1.0 eq. 55.3 mol). Formic acid (630 g, 0.25 eq. 13.8 mol.) was charged into the reactor. Charge NH₂NHBoc (10.1 kg, 1.3 eq., 76.5 mol) into the reactor in the EtOH solution. Heat the mixture at 40-50° C. for 14 h. Concentrate the mixture to 1.5-2.5 vol. Charge IPAc (109.2 kg, 10 vol., 8.9 wt/wt) into the reactor. Concentrated the mixture to 3.0-3.5 vol. Take sample to check the residual of EtOH. (Spec: ≤1.0%). Stir the mixture at 50-60° C. for 10-20 min. Add n-heptane (67.3 kg, 8.0 vol. 5.4 wt/wt) into the reactor via 5 min. Stir the mixture at 50-60° C. for 1-3 h. Cool down to 10-20° C. via 5 h. Stir the mixture at 10-20° C. for 13-20 h. Take sample to check the ratio of n-heptane and IPAc. (Spec: 2.6˜3.5 (n-hetane/IPAc from 3/1 to 5/1)). Filter and wash wet cake with n-heptane (21.6 kg, 2.5 vol., 1.75 wt/wt), 17.8 kg (1.45 wt/wt) of wet A8 was obtained. Dry 15.5 kg (1.26 wt/wt) wet cake under 40-50° C. for 10-20 h. Obtain the solid. (13.7 kg, 40.7 mol, 81.2% yield for 2 steps, 99.8% HPLC purity, >99.9% chiral purity, 98.8% qNMR assay, the residual of EtOH, n-heptane and IPAc was separately 817 ppm, 834 ppm and 491 ppm. The other solvent was N.D, the residual of enzyme was 26 ppm. The KF was 0.10%. The ROI was 0.08%).

Alternative 2:

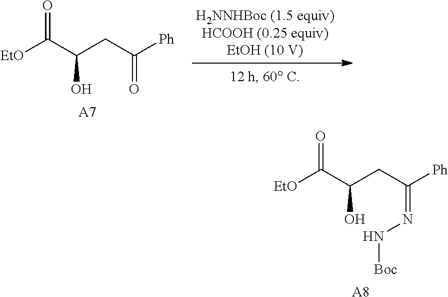

To a 30 L reactor containing a solution of A7 in ethanol at Tᵢ=25° C. was charged formic acid (0.05 kg, 1024.8 mmol, 0.25 equiv), tert-butyl carbazate (0.90 kg, 6148.8 mmol, 1.5 equiv), and ethanol (1.31 kg). The reaction mixture was heated to Tᵢ=60° C. overnight (A8 versus A7: >95:5 (A %:A %)). The reaction mixture was concentrated to 5 V in vacuo (˜50° C. internal ˜275 mbar). n-Heptane (3.26 kg) was added and contents of Reactor 2 were concentrated to 5 V in vacuo through vacuum distillation. n-Heptane (3.26 kg) was added and contents of the 30 L Reactor were concentrated to 5 V in vacuo through vacuum distillation. To a suspension n-heptane (3.40 kg) was added and stirred at 45-50° C. Then MTBE (3.00 kg) was added. The mixture was cooled to 0-5° C. over 1 h and additional n-heptane (5.11 kg) was added over 1 h. The slurry was aged overnight transferred to a filter dryer and filtered. The cake was washed with n-heptane (5.11 kg) then dried under a stream of nitrogen at 25° C. over the weekend. A fine off-white solid was obtained in 67% yield with 96.2 A % purity by HPLC and KF=0.69 wt %.

Additional product (15% yield) was recovered from the wall cake by isolation and recrystallization using the procedure below. To 5 L reactor was charged slurry of A8 in MTBE (1.6 kg). Contents of the reactor were heated to 50° C. and stirred until dissolution was achieved. Then temperature was brought to 45° C., seeds were added (1 wt %), then contents were aged for 30 min at 40° C. n-Heptane (2.41 kg) was added over 1.5 h followed by cooling at 5° C. over 1 h. Final slurry was aged for 1 h at 5° C. Solids were filtered using 3 L glass filter. Cake was washed with n-heptane (0.602 kg), and then dried at 25° C. under nitrogen flow overnight. A fine off-white solid of A8 was obtained in 85% recovery with 96.2 A % purity by HPLC (HPLC 96.2 A %, 98.5:1.5 dr, Chiral HPLC >99% ee).

Exemplary compounds prepared by the above processes are described herein together with ¹H NMR data. In certain examples, chiral column retention times (min) are provided for certain stereoisomers. Unless otherwise specified, the stereochemistry shown in each structure represents relative configuration of a single stereoisomer, and absolute configuration (i.e., “R” and/or “S”) is arbitrarily assigned.

All of the U.S. patents, U.S. patent application publications, U.S. patent applications, foreign patents, foreign patent applications, international and non-patent publications referred to in this specification are incorporated herein by reference in their entireties.

Although the foregoing embodiments of the present invention have been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

We claim:

1. A process for the preparation of a chiral bicylic ketone compound of formula (I), or a stereoisomer thereof: 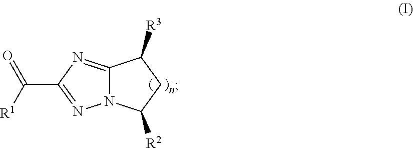
or a pharmaceutically acceptable salt thereof, wherein:
R¹ is selected from the group consisting of C₁-C₆ alkyl, C₃-C₆ cycloalkyl, C₁-C₆ alkoxy, C₁-C₆ haloalkyl, C₁-C₆ haloalkoxy, C₁-C₆ alkyl-N(R^{N})₂, phenyl, benzyl, 4 to 8 membered heterocyclyl and 5 to 6 membered heteroaryl, wherein R¹ is bound to the adjacent carbonyl by a carbon atom and R¹ is optionally substituted by one or two substituents selected from the group consisting of F, Cl, Br, C₁-C₆ alkyl, C₃-C₆ cycloalkyl, C₁-C₆ alkoxy, C₁-C₆ haloalkyl, C₁-C₆ haloalkoxy, C₁-C₆ alkyl-N(R^{N})₂, hydroxyl, hydroxymethyl, cyano, cyanomethyl, cyanoethyl, C(O)C₁-C₆ alkyl, phenyl, benzyl, CH₂—(C₃-C₆ cycloalkyl), 5 to 6 membered heteroaryl, and CH₂-(5 to 6 membered heteroaryl);
each R^{N} is independently selected from the group consisting of H, C₁-C₆ alkyl, C₃-C₆ cycloalkyl, C₁-C₆ alkoxy, and C₁-C₆ haloalkyl; or two R^{N} may together with the adjacent N form a 4-6 membered ring;
R² is selected from the group consisting of H, C₁-C₆ alkyl, C₁-C₆ haloalkyl, C₃-C₆ cycloalkyl, C₁-C₆ alkoxy, C₁-C₆ haloalkoxy, C₁-C₆ thioalkyl, phenyl, benzyl, CH₂—(C₃-C₆ cycloalkyl), CH₂CH₂—(C₃-C₆ cycloalkyl), CH₂-(4 to 6 membered heterocyclyl), CH₂CH₂-(4 to 6 membered heterocyclyl), 5 to 6 membered heteroaryl, and CH₂-(5 to 6 membered heteroaryl); wherein when a phenyl ring is present it may be substituted by 1 to 3 substituents selected from the group consisting of halogen, C₁-C₄ alkyl, C₁-C₄ haloalkyl, C₁-C₄ alkoxy, C₁-C₄ haloalkoxy, and cyano;
R³ is selected from the group consisting of D, halogen, OH, CN, C₁-C₄ alkyl, C₁-C₄ haloalkyl, cyclopropyl, C₁-C₄ alkoxy and C₁-C₄ haloalkoxy; and
n is 1, 2 or 3;
the process comprising:
(a) contacting a compound of chiral N-amino lactam formula p, or a stereoisomer thereof: 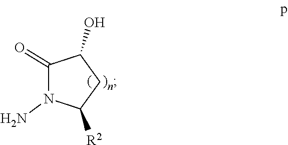
or a salt thereof, in the presence of an acid additive and an alcohol solvent with an imidate compound of formula c: 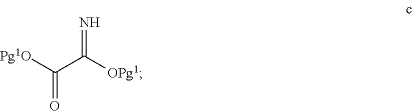
or a salt thereof, to form a chiral bicyclic triazole compound of formula x, or a stereoisomer thereof: 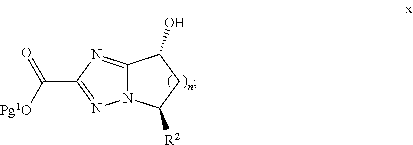
or a salt thereof;
wherein:
Pg¹ is an optional hydroxyl protecting group and may be the same or different on each occurrence; and
the chiral bicyclic triazole compound of formula x, or the stereoisomer thereof, is an intermediate compound in the preparation of the chiral bicyclic ketone compound of formula (I), or the stereoisomer thereof.

2. The process of claim 1, wherein the acid additive is acetic acid and the organic solvent is EtOH.

3. The process of any one of claims 1-2, wherein a yield of the chiral bicyclic triazole compound of formula x of step (a) is at least 80%.

4. The process of claim 3, wherein the yield is at least 98%.

5. The process of any one of claims 1-4, further comprising:
(b) deoxyhalogenating the chiral bicyclic triazole compound of formula x, or the stereoisomer thereof, in the presence of a halogenating agent to form a chiral halogenated bicyclic compound of formula y, or a stereoisomer thereof: 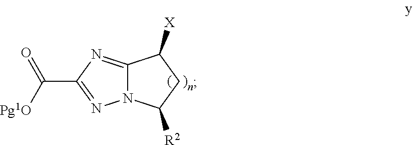
or a salt thereof, wherein X is halogen.

6. The process of claim 5, wherein the halogenating agent is PBSF or 2-pyridinesulfonyl fluoride.

7. The process of any one of claims 5-6, wherein the deoxyhalogenating step (b) is performed in the presence of an organic base and an organic solvent.

8. The process of claim 7, wherein the organic base is N,N-diisopropylethylamine and the organic solvent is acetonitrile.

9. The process of any one of claims 5-8, wherein the deoxyhalogenating step (b) is further performed in the presence of an additive acting as a fluoride source.

10. The process of claim 9, wherein the additive is triethylamine trihydrofluoride.

11. The process of any one of claims 5-10, further comprising:
(c) contacting the chiral halogenated bicyclic compound of formula y, or the stereoisomer thereof, with an acid in the presence of an ethereal solvent/water mixture to form a halogenated bicyclic carboxylic acid compound of formula z, or a stereoisomer thereof: 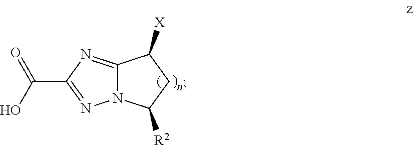
or a salt thereof.

12. The process of claim 11, wherein the ethereal solvent/water mixture is a THF/water mixture and the acid is HCl.

13. The process of any one of claims 11-12, further comprising:
(d) contacting the halogenated bicyclic carboxylic acid compound of formula z, or the stereoisomer thereof, with a compound of formula aa: 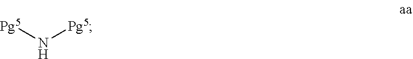
or a salt thereof, in the presence of a coupling agent to form a chiral bicyclic amide compound of formula bb or a stereoisomer thereof: 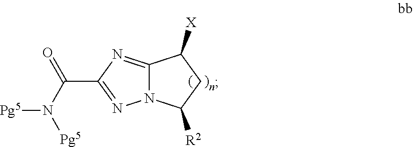
or salt thereof, wherein each Pg⁵ is an amine protecting group and may be the same or different on each occurrence.

14. The process of claim 13, wherein the coupling agent is EDCI.

15. The process of any one of claims 13-14, further comprising:
(e) contacting the chiral bicyclic amide compound of formula bb or stereoisomer thereof, with a compound of formula cc:
R¹—MgBr_{cc};
or a salt thereof, to form a chiral bicyclic ketone compound dd, or stereoisomer thereof: 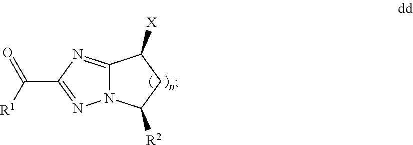
or salt thereof.

16. The process of claim 15, further comprising adding seeds of the chiral bicyclic ketone compound of formula dd.

17. The process of any one of claims 1-16, wherein the chiral bicyclic ketone compound of formula (I) is a compound selected from the group consisting of: 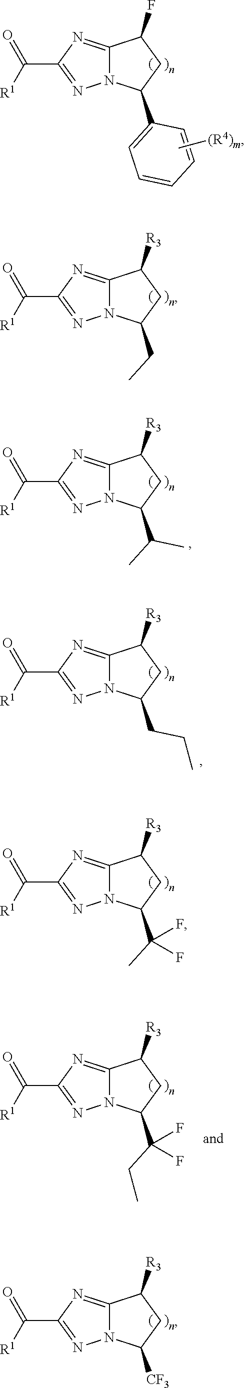
or a pharmaceutically acceptable salt thereof, wherein:
each R⁴ is selected from the group consisting of H, F, Cl, C₁-C₆ alkyl, C₁-C₆ haloalkyl, C₁-C₆ alkoxy and C₁-C₆ haloalkoxy; and
m is 0, 1, 2 or 3.

18. The process of any one of claims 1-17, wherein the chiral bicyclic ketone compound of formula (I) is: 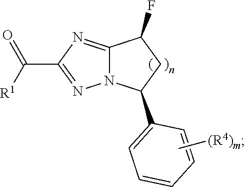
or a pharmaceutically acceptable salt thereof.

19. The process of claim 1, wherein the stereoisomer of the chiral bicyclic ketone compound of formula (I) is a compound of formula (II): 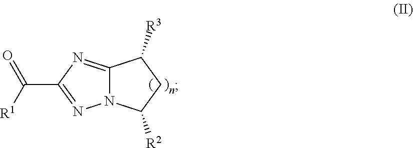
or a pharmaceutically acceptable salt thereof.

20. The process of claim 1 or 19, wherein the compound of formula (II) is a compound selected from the group consisting of: 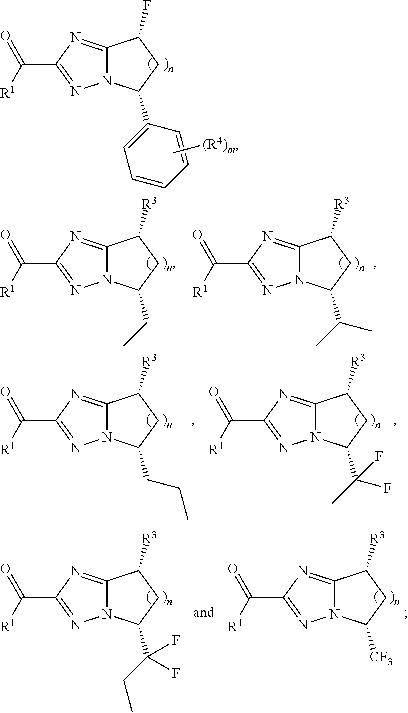
or a pharmaceutically acceptable salt thereof, wherein:
each R⁴ is selected from the group consisting of H, F, Cl, C₁-C₆ alkyl, C₁-C₆ haloalkyl, C₁-C₆ alkoxy and C₁-C₆ haloalkoxy; and
m is 0, 1, 2 or 3.

21. The process of claim 20, wherein the compound of formula (II) is: 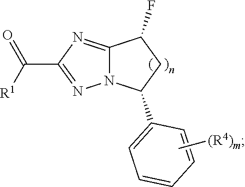
or a pharmaceutically acceptable salt thereof.

22. The process of any one of claims 17, 18, 20 and 21, wherein m is 0.

23. The process of any one of claims 1-22, wherein R¹ is cyclopropyl.

24. A process for the preparation of a chiral N-amino lactam compound of formula p, or a stereoisomer thereof: 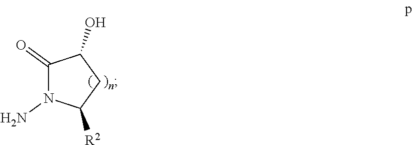
or a salt thereof; wherein:
R² is selected from the group consisting of H, C₁-C₆ alkyl, C₁-C₆ haloalkyl, C₃-C₆ cycloalkyl, C₁-C₆ alkoxy, C₁-C₆ haloalkoxy, C₁-C₆ thioalkyl, phenyl, benzyl, CH₂—(C₃-C₆ cycloalkyl), CH₂CH₂—(C₃-C₆ cycloalkyl), CH₂-(4 to 6 membered heterocyclyl), CH₂CH₂-(4 to 6 membered heterocyclyl), 5 to 6 membered heteroaryl, and CH₂-(5 to 6 membered heteroaryl); wherein when a phenyl ring is present it may be substituted by 1 to 3 substituents selected from the group consisting of halogen, C₁-C₄ alkyl, C₁-C₄ haloalkyl, C₁-C₄ alkoxy, C₁-C₄ haloalkoxy, and cyano; and
n is 1, 2 or 3;
the process comprising:
(a) reacting a chiral hydroxydicarboxylic acid compound of formula d or a stereoisomer 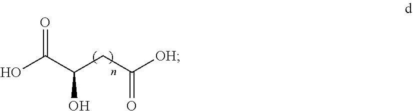
or a salt thereof, in the presence of organic acid chloride of formula e: 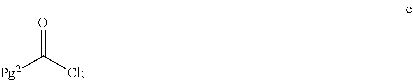
or a salt thereof, to form a chiral carboxylic cyclic anhydride compound of formula f or a stereoisomer thereof: 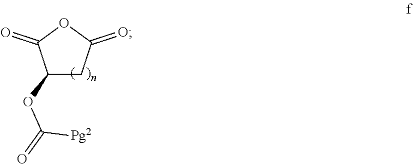
or a salt thereof; and
(b) reacting a protected hydrazone compound of formula 1, or a stereoisomer thereof: 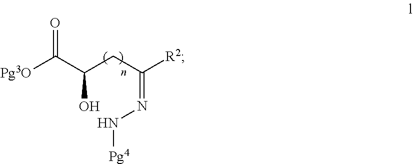
or a salt thereof, in the presence of an acid additive, to form a chiral hydroxy ester hydrazine compound of formula m or a stereoisomer thereof: 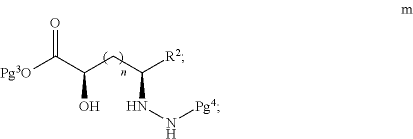
or a salt thereof; wherein:
Pg² is optionally substituted C₁-C₆ alkyl, C₃-C₆ cycloalkyl or aryl;
Pg³ is an optional hydroxyl protecting group and may be the same or different on each occurrence;
Pg⁴ is an optional amine protecting group and may be the same or different on each occurrence;
the chiral carboxylic cyclic anhydride compound of formula f, or the stereoisomer thereof, is an intermediate in the preparation of the protected hydrazone compound of formula 1, or the stereoisomer thereof; and
the chiral hydroxy ester hydrazine compound of formula m or the stereoisomer thereof, is an intermediate in the preparation of the chiral N-amino lactam compound of formula p, or the stereoisomer thereof.

25. The process of claim 24, wherein a yield of the chiral carboxylic cyclic anhydride compound of formula f of step (a) is at least 80%.

26. The process of claim 25, wherein the yield is at least 93%.

27. The process of any one of claims 24-26, wherein a yield of the chiral hydroxy ester hydrazine compound of formula m of step (b) is at least 70%.

28. The process of claim 27, wherein the yield is at least 90%.

29. The process of any one of claims 24-28, wherein a diasteroisomeric ratio of the chiral hydroxy ester hydrazine compound of formula m of step (b) to its diastereomer is at least 10:1.

30. The process of claim 29, wherein the diasteroisomeric ratio is at least 14:1.

31. The process of any one of claims 24-30, wherein the acid additive is AcOH.

32. The process of any one of claims 24-31, further comprising:
(c) contacting the chiral carboxylic cyclic anhydride compound of formula f or the stereoisomer thereof, with a reactive arene compound to form a compound of formula h, or a stereoisomer thereof: 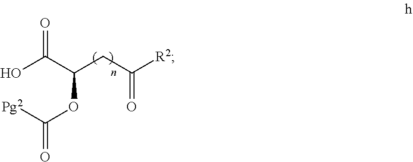
or a salt thereof.

33. The process of claim 32, wherein the reactive arene compound is benzene.

34. The process of any one of claims 32-33, wherein the contacting step (c) is performed in the presence of a Lewis acid in an organic solvent.

35. The process of claim 34, wherein the Lewis acid is AlCl₃ and the organic solvent is CH₂Cl₂.

36. The process of claim 32, further comprising:
(d) reacting the compound of formula h or the stereoisomer thereof, in an alcohol solvent of formula i:
Pg³-OHᵢ;
to form a compound of formula j, or a stereoisomer thereof: 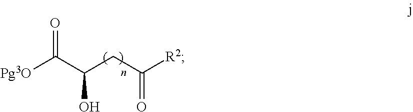
or a salt thereof.

37. The process of claim 36, wherein the alcohol solvent of formula i is EtOH.

38. The process of any one of claims 36-37, wherein the reacting step (d) is performed in the presence of an acid.

39. The process of claim 38, wherein the acid is H₂SO₄.

40. The process of any one of claims 36-39, further comprising:
(e) contacting the compound of formula j, or the stereoisomer thereof, with a hydrazine compound of formula k:
H₂NNH-Pg⁴ₖ;
or a salt thereof, to form the protected hydrazone compound of formula 1, or the stereoisomer thereof, or the salt thereof.

41. The process of claim 40, wherein the contacting step (e) is performed in the presence of an acid additive.

42. The process of claim 41, wherein the acid additive is formic acid.

43. The process of any one of claims 40-42, further comprising:
(f) reacting the protected hydrazone compound of formula 1, or the stereoisomer thereof, to form a chiral protected N-amino lactam compound of formula n, or a stereoisomer thereof: 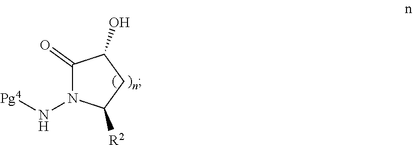
or a salt thereof;
(g) deprotecting the chiral protected N-amino lactam compound of formula n or the stereoisomer thereof, to form a salt compound of formula o or a stereoisomer thereof: 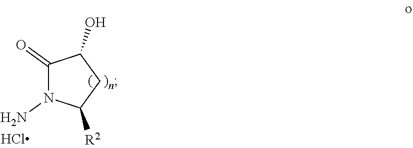
and
(h) reacting the salt compound of formula o or the stereoisomer thereof, in the presence of a base to form the chiral N-amino lactam compound of formula p, or the stereoisomer thereof.

44. The process of claim 43, wherein the base is NaOH in an aqueous base solution.

45. The process of any one of claims 24-44, wherein the optional amine protecting group Pg⁴ is Boc.

46. The process of any one of claims 1-45, wherein n is 1.

47. A process for the preparation of an imidate salt compound of formula b: 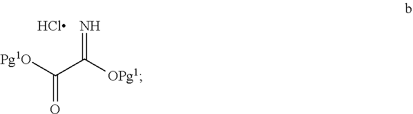
(a) reacting a cyanoformate compound of formula a: 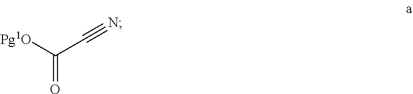
in the presence of an anhydrous acid source in an alcohol solvent to form the imidate salt compound of formula b, wherein the anhydrous acid source is TMSCl, the acid is HCl, and Pg¹ is an optional hydroxyl protecting group and may be the same or different on each occurrence.

48. The process of claim 47, wherein the alcohol solvent is EtOH in MTBE.

49. The process of any one of claims 47-48, wherein a yield of the imidate salt compound of formula b of step (a) is at least 65%.

50. The process of claim 49, wherein the yield is at least 78%.

51. A process for the preparation of a hydroxyketoester compound of formula j, or a stereoisomer thereof: 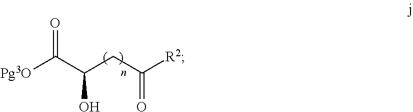
wherein:
R² is selected from the group consisting of H, C₁-C₆ alkyl, C₁-C₆ haloalkyl, C₃-C₆ cycloalkyl, C₁-C₆ alkoxy, C₁-C₆ haloalkoxy, C₁-C₆ thioalkyl, phenyl, benzyl, CH₂—(C₃-C₆ cycloalkyl), CH₂CH₂—(C₃-C₆ cycloalkyl), CH₂-(4 to 6 membered heterocyclyl), CH₂CH₂-(4 to 6 membered heterocyclyl), 5 to 6 membered heteroaryl, and CH₂-(5 to 6 membered heteroaryl); wherein when a phenyl ring is present it may be substituted by 1 to 3 substituents selected from the group consisting of halogen, C₁-C₄ alkyl, C₁-C₄ haloalkyl, C₁-C₄ alkoxy, C₁-C₄ haloalkoxy, and cyano; and
n is 1, 2 or 3;
the process comprising:
(a) reacting a diketoester compound of formula hh: 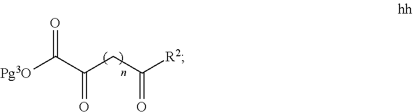
in the presence of a ketoreductase (KRED) to form the hydroxyketoester compound of formula, or a stereoisomer thereof, wherein:
Pg³ is an optional hydroxyl protecting group and may be the same or different on each occurrence.

52. The process of claim 51, further comprising:
(b) reacting the hydroxyketoester compound of formula j, or a stereoisomer thereof, to form a protected hydrazone compound of formula 1, or a stereoisomer thereof: 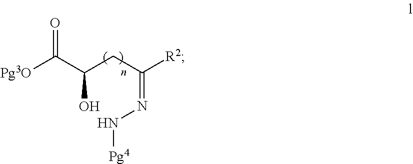
or a salt thereof.

53. The process of claim 51, wherein a yield of the hydroxyketoester compound of formula j of step (a) is at least 80%.

54. The process of claim 52, wherein a yield of protected hydrazone compound of formula 1 of step (b) is at least 80%.

55. The process of any one of claims 51-54, wherein the KRED is highly stereoselective.

56. The process of any one of claims 51-55, wherein a stereomeric excess of the hydroxyketoester compound of formula j of step (a) is at least 90%.

57. The process of any one of claims of claims 51-56, wherein the KRED is provided in an immobilized form or in form of a whole cell.

58. The process of any one of claims 51-57, wherein the KRED is an engineered ketoreductase.

59. The process of claim 58, wherein the engineered ketoreductase is ADH-114 or 1-200-0-16.

60. The process of any one of claims 51-59, further comprising the presence of a cofactor.

61. The process of claim 60, wherein the cofactor is NAD, NADH, NADP or NADPH.

62. The process of any one of claims 51-61, further comprising the presence of a coenzyme.

63. The process of claim 62, wherein the coenzyme is a glucose dehydrogenase.

64. The process of claim 63, wherein the glucose dehydrogenase is GDH-105 or 1-030-0-05.

65. The process of any one of claims 51-61, further comprising the presence of an alcohol.

66. The process of claim 65, wherein the alcohol is a secondary alcohol.

67. The process of claim 66, wherein the secondary alcohol is isopropanol.

68. The process of any one of claims 1-67, wherein at least one step of the process is scalable to at least a kilogram scale.

69. A compound, or a pharmaceutically acceptable salt thereof, prepared according to a process of any one of claims 1-68.

70. The invention as hereinbefore described.
